SEQUENCE LISTING (127 sequences)

SEQ ID NO: 1 - RNA, 22 nt, Homo sapiens
aacccguagauccgaucuugug

SEQ ID NO: 2 - RNA, 22 nt, Homo sapiens
uaacacugucugguaaagaugg

SEQ ID NO: 3 - RNA, 21 nt, Homo sapiens
ucgaggagcucacagucuagu

SEQ ID NO: 4 - RNA, 23 nt, Homo sapiens
uuuggcacuagcacauuuuugcu

SEQ ID NO: 5 - RNA, 22 nt, Homo sapiens
ucccugagacccuaacuuguga

SEQ ID NO: 6 - RNA, 22 nt, Homo sapiens
uauggcacugguagaauucacu

SEQ ID NO: 7 - RNA, 18 nt, Homo sapiens
aucccaccucugccacca

SEQ ID NO: 8 - RNA, 22 nt, Homo sapiens
uuugguccccuucaaccagcug

SEQ ID NO: 9 - RNA, 22 nt, Homo sapiens
uaauacugucugguaaaaccgu

SEQ ID NO: 10 - RNA, 22 nt, Homo sapiens
ggugcagugcugcaucucuggu

SEQ ID NO: 11 - RNA, 22 nt, Homo sapiens
ugaccgauuucuccugguguuc

SEQ ID NO: 12 - RNA, 22 nt, Homo sapiens
uagcaccauuugaaaucgguua

SEQ ID NO: 13 - RNA, 21 nt, Homo sapiens
ucagugcaugacagaacuugg

SEQ ID NO: 14 - RNA, 22 nt, Homo sapiens
aacccguagauccgaacuugug

SEQ ID NO: 15 - RNA, 21 nt, Homo sapiens
uucacaguggcuaaguucugc

SEQ ID NO: 16 - RNA, 21 nt, Homo sapiens
cagcagcacacugugguuugu

SEQ ID NO: 17 - RNA, 21 nt, Homo sapiens
cuauacaaucuacugucuuuc

SEQ ID NO: 18 - RNA, 22 nt, Homo sapiens
cuauacaaccuacugccuuccc

SEQ ID NO: 19 - RNA, 22 nt, Homo sapiens
cuauacgaccugcugccuuucu

SEQ ID NO: 20 - RNA, 22 nt, Homo sapiens
uggaauguaaagaaguauguau

SEQ ID NO: 21 - RNA, 21 nt, Homo sapiens
uacaguacugugauaacugaa

SEQ ID NO: 22 - RNA, 21 nt, Homo sapiens
uaaagugcugacagugcagau

SEQ ID NO: 23 - RNA, 23 nt, Homo sapiens
uacccuguagauccgaauuugug

SEQ ID NO: 24 - RNA, 23 nt, Homo sapiens
uacccuguagaaccgaauuugug

SEQ ID NO: 25 - RNA, 21 nt, Homo sapiens
aagcauucuuucauugguugg

SEQ ID NO: 26 - RNA, 21 nt, Homo sapiens
ccgucgccgccacccgagccg

SEQ ID NO: 27 - RNA, 21 nt, Homo sapiens
gugccagcugcagugggggag

SEQ ID NO: 28 - RNA, 22 nt, Homo sapiens
ucggccugaccacccaccccac

SEQ ID NO: 29 - RNA, 22 nt, Homo sapiens
cguguucacagcggaccuugau

SEQ ID NO: 30 - RNA, 22 nt, Homo sapiens
cggaugagcaaagaaagugguu

SEQ ID NO: 31 - RNA, 22 nt, Homo sapiens
ucggauccgucugagcuuggcu

SEQ ID NO: 32 - RNA, 17 nt, Homo sapiens
ucccaccgcugccaccc

SEQ ID NO: 33 - RNA, 22 nt, Homo sapiens
aagcccuuaccccaaaaagcau

SEQ ID NO: 34 - RNA, 22 nt, Homo sapiens
cagugcaauguuaaaagggcau

SEQ ID NO: 35 - RNA, 22 nt, Homo sapiens
uuugguccccuucaaccagcua

SEQ ID NO: 36 - RNA, 23 nt, Homo sapiens
uauggcuuuucauuccuauguga

SEQ ID NO: 37 - RNA, 23 nt, Homo sapiens
acuccauuuguuuugaugaugga

SEQ ID NO: 38 - RNA, 22 nt, Homo sapiens
caucaucgucucaaaugagucu

SEQ ID NO: 39 - RNA, 23 nt, Homo sapiens
uuauugcuuaagaauacgcguag

SEQ ID NO: 40 - RNA, 22 nt, Homo sapiens
ucuacagugcacgugucuccag

SEQ ID NO: 41 - RNA, 21 nt, Homo sapiens
uaccacaggguagaaccacgg

SEQ ID NO: 42 - RNA, 22 nt, Homo sapiens
caucuuccaguacaguguugga

SEQ ID NO: 43 - RNA, 21 nt, Homo sapiens
ugagaugaagcacuguagcuc

SEQ ID NO: 44 - RNA, 20 nt, Homo sapiens
uacaguauagaugauguacu

SEQ ID NO: 45 - RNA, 23 nt, Homo sapiens
guccaguuuucccaggaaucccu

SEQ ID NO: 46 - RNA, 23 nt, Homo sapiens
ucuggcuccgugucuucacuccc

SEQ ID NO: 47 - RNA, 22 nt, Homo sapiens
ucucccaacccuuguaccagug

SEQ ID NO: 48 - RNA, 21 nt, Homo sapiens
cuagacugaagcuccuugagg

SEQ ID NO: 49 - RNA, 22 nt, Homo sapiens
uagguuauccguguugccuucg

SEQ ID NO: 50 - RNA, 24 nt, Homo sapiens
uuuggcaaugguagaacucacacu

SEQ ID NO: 51 - RNA, 22 nt, Homo sapiens
ucgugucuuguguugcagccgg

SEQ ID NO: 52 - RNA, 23 nt, Homo sapiens
uaaggugcaucuagugcagauag

SEQ ID NO: 53 - RNA, 23 nt, Homo sapiens
acugcccuaagugcuccuucugg

SEQ ID NO: 54 - RNA, 21 nt, Homo sapiens
uagcagcacagaaauauuggc

SEQ ID NO: 55 - RNA, 23 nt, Homo sapiens
cccaguguucagacuaccuguuc

SEQ ID NO: 56 - RNA, 22 nt, Homo sapiens
acaguagucugcacauugguua

SEQ ID NO: 57 - RNA, 23 nt, Homo sapiens
ugugcaaaucuaugcaaaacuga

SEQ ID NO: 58 - RNA, 22 nt, Homo sapiens
uaacacugucugguaacgaugu

SEQ ID NO: 59 - RNA, 22 nt, Homo sapiens
uaauacugccugguaaugauga

SEQ ID NO: 60 - RNA, 23 nt, Homo sapiens
uaauacugccggguaaugaugga

SEQ ID NO: 61 - RNA, 22 nt, Homo sapiens
gugaaauguuuaggaccacuag

SEQ ID NO: 62 - RNA, 22 nt, Homo sapiens
uucccuuugucauccuaugccu

SEQ ID NO: 63 - RNA, 22 nt, Homo sapiens
uccuucauuccaccggagucug

SEQ ID NO: 64 - RNA, 22 nt, Homo sapiens
uagcuuaucagacugauguuga

SEQ ID NO: 65 - RNA, 21 nt, Homo sapiens
caacaccagucgaugggcugu

SEQ ID NO: 66 - RNA, 22 nt, Homo sapiens
cugugcgugugacagcggcuga

SEQ ID NO: 67 - RNA, 22 nt, Homo sapiens
acagcaggcacagacaggcagu

SEQ ID NO: 68 - RNA, 21 nt, Homo sapiens
uugugcuugaucuaaccaugu

SEQ ID NO: 69 - RNA, 21 nt, Homo sapiens
agcuacaucuggcuacugggu

SEQ ID NO: 70 - RNA, 21 nt, Homo sapiens
caagucacuagugguuccguu

SEQ ID NO: 71 - RNA, 23 nt, Homo sapiens
caggcagugacuguucagacguc

SEQ ID NO: 72 - RNA, 22 nt, Homo sapiens
cacuagauugugagcuccugga

SEQ ID NO: 73 - RNA, 22 nt, Homo sapiens
gaggguuggguggaggcucucc

SEQ ID NO: 74 - RNA, 22 nt, Homo sapiens
ugguuuaccgucccacauacau

SEQ ID NO: 75 - RNA, 22 nt, Homo sapiens
uagcaccaucugaaaucgguua

SEQ ID NO: 76 - RNA, 23 nt, Homo sapiens
uagcaccauuugaaaucaguguu

SEQ ID NO: 77 - RNA, 22 nt, Homo sapiens
uguaaacauccucgacuggaag

SEQ ID NO: 78 - RNA, 22 nt, Homo sapiens
cuuucagucggauguuugcagc

SEQ ID NO: 79 - RNA, 23 nt, Homo sapiens
uguaaacauccuacacucucagc

SEQ ID NO: 80 - RNA, 22 nt, Homo sapiens
cuuucagucggauguuuacagc

SEQ ID NO: 81 - RNA, 21 nt, Homo sapiens
aggcaagaugcuggcauagcu

SEQ ID NO: 82 - RNA, 19 nt, Homo sapiens
gagggcggguggaggagga

SEQ ID NO: 83 - RNA, 20 nt, Homo sapiens
uuggccauggggcugcgcgg

SEQ ID NO: 84 - RNA, 22 nt, Homo sapiens
uauugcacauuacuaaguugca

SEQ ID NO: 85 - RNA, 20 nt, Homo sapiens
acugccccaggugcugcugg

SEQ ID NO: 86 - RNA, 21 nt, Homo sapiens
gugcauuguaguugcauugca

SEQ ID NO: 87 - RNA, 22 nt, Homo sapiens
uggcagugucuuagcugguugu

SEQ ID NO: 88 - RNA, 20 nt, Homo sapiens
gaaucggaaaggaggcgccg

SEQ ID NO: 89 - RNA, 23 nt, Homo sapiens
uagccuucagaucuugguguuuu

SEQ ID NO: 90 - RNA, 22 nt, Homo sapiens
gggaggugugaucucacacucg

SEQ ID NO: 91 - RNA, 22 nt, Homo sapiens
acucaaaaugggggcgcuuucc

SEQ ID NO: 92 - RNA, 21 nt, Homo sapiens
aucauagaggaaaauccacgu

SEQ ID NO: 93 - RNA, 21 nt, Homo sapiens
aacauagaggaaauuccacgu

SEQ ID NO: 94 - RNA, 22 nt, Homo sapiens
aucacacaaaggcaacuuuugu

SEQ ID NO: 95 - RNA, 20 nt, Homo sapiens
acuggacuuggagucagaaa

SEQ ID NO: 96 - RNA, 21 nt, Homo sapiens
ugguagacuauggaacguagg

SEQ ID NO: 97 - RNA, 23 nt, Homo sapiens
aaugacacgaucacucccguuga

SEQ ID NO: 98 - RNA, 17 nt, Homo sapiens
ccugagaaaagggccaa

SEQ ID NO: 99 - RNA, 21 nt, Homo sapiens
ugucuugcaggccgucaugca

SEQ ID NO: 100 - RNA, 17 nt, Homo sapiens
ccaguuuucccaggauu

SEQ ID NO: 101 - RNA, 22 nt, Homo sapiens
aucaugaugggcuccucggugu

SEQ ID NO: 102 - RNA, 22 nt, Homo sapiens
uggcaguguauuguuagcuggu

SEQ ID NO: 103 - RNA, 22 nt, Homo sapiens
aggcaguguauuguuagcuggc

SEQ ID NO: 104 - RNA, 22 nt, Homo sapiens
aaaccguuaccauuacugaguu

SEQ ID NO: 105 - RNA, 22 nt, Homo sapiens
aggacuggacucccggcagccc

SEQ ID NO: 106 - RNA, 24 nt, Homo sapiens
gaugcgccgcccacugccccgcgc

SEQ ID NO: 107 - RNA, 20 nt, Homo sapiens
ccauggaucuccaggugggu

SEQ ID NO: 108 - RNA, 22 nt, Homo sapiens
ugaaggucuacugugugccagg

SEQ ID NO: 109 - RNA, 22 nt, Homo sapiens
aaacaaacauggugcacuucuu

SEQ ID NO: 110 - RNA, 21 nt, Homo sapiens
guuucaccauguuggucaggc

SEQ ID NO: 111 - RNA, 21 nt, Homo sapiens
aaagugcuuccuuuuugaggg

SEQ ID NO: 112 - RNA, 21 nt, Homo sapiens
gcuaguccugacucagccagu

SEQ ID NO: 113 - RNA, 22 nt, Homo sapiens
cacgcucaugcacacacccaca

SEQ ID NO: 114 - RNA, 22 nt, Homo sapiens
aagauguggaaaaauuggaauc

SEQ ID NO: 115 - RNA, 22 nt, Homo sapiens
agucauuggaggguuugagcag

SEQ ID NO: 116 - RNA, 23 nt, Homo sapiens
aaacucuacuuguccuucugagu

SEQ ID NO: 117 - RNA, 22 nt, Homo sapiens
gacuauagaacuuucccccuca

SEQ ID NO: 118 - RNA, 22 nt, Homo sapiens
gguggcccggccgugccugagg

SEQ ID NO: 119 - RNA, 21 nt, Homo sapiens
uccgguucucagggcuccacc

SEQ ID NO: 120 - RNA, 23 nt, Homo sapiens
uggaagacuagugauuuuguugu

SEQ ID NO: 121 - RNA, 23 nt, Homo sapiens
aaggagcuuacaaucuagcuggg

SEQ ID NO: 122 - RNA, 22 nt, Homo sapiens
agggacgggacgcggugcagug

SEQ ID NO: 123 - RNA, 23 nt, Homo sapiens
caaagugcuguucgugcagguag

SEQ ID NO: 124 - RNA, 22 nt, Homo sapiens
ugucuacuacuggagacacugg

SEQ ID NO: 125 - RNA, 21 nt, Homo sapiens
aaggcagggcccccgcucccc

SEQ ID NO: 126 - DNA, 60 bp, Homo sapiens
agtgatgatgaccccaggtaactctgagtgtgtcgctgatgccatcaccgcagcgctctg

SEQ ID NO: 127 - DNA, 299 bp, Homo sapiens
gccgggcgcggtggcgcgtgcctgtagtcccagctactcgggaggctgaggctggaggatcgcttgagtccaggagttctgggctgtagtgcgctatgccgatcgggtgtccgcactaagttcggcatcaatatggtgacctcccgggagcgggggaccaccaggttgcctaaggaggggtgaaccggcccaggtcggaaacggagcaggtcaaaactcccgtgctgatcagtagtgggatcgcgcctgtgaatagccactgcactccagcctgggcaacatagcgagaccccgtctct

(19) United States
(12) Patent Application Publication
(10) Pub. No.: US 2022/0372578 A1
(43) Pub. Date: Nov. 24, 2022

(54) URINE MIRNA FINGERPRINT FOR DETECTING BLADDER AND UROTHELIAL CARCINOMA AND APPLICATION THEREOF

(71) Applicant: BIOVUE TECHNOLOGY LTD, Shanghai (CN)

(72) Inventors: Ruoying TAN, Shanghai (CN); Hongfei WU, Shanghai (CN); Fengwu ZHANG, Shanghai (CN); Hailong WANG, Suzhou (CN)

(21) Appl. No.: 17/636,902

(22) PCT Filed: Aug. 18, 2020
(86) PCT No.: PCT/CN2020/109710

§ 371 Date: Feb. 21, 2022

(30) Foreign Application Priority Data
Aug. 19, 2019 (CN) 201910776393.8

(51) Int. Cl. C12Q 1/6886; G16B 25/10; G16B 40/20

(52) U.S. Cl. CPC C12Q 1/6886; G16B 25/10; G16B 40/20; C12Q 2600/178; C12Q 2600/158; C12Q 2600/112; C12Q 1/6837

(57) ABSTRACT

Provided is an application of a miRNA fingerprint in the diagnosis and treatment of human bladder and urothelial carcinoma (comprising bladder cancer, renal pelvic carcinoma, ureter cancer, urinary outflow tract cancer, and the like). A plurality of fingerprints consisting of miRNAs can effectively distinguish a urine sample of bladder and urothelial carcinoma from a urine sample of non-bladder and urothelial carcinoma, and has high sensitivity and strong specificity. The fingerprints can be effectively used for the detection and the early diagnosis and screening of bladder and urothelial carcinoma and the screening of drugs for bladder and urothelial carcinoma.

### FIELD OF THE INVENTION

The present disclosure relates to the field of biomedicine, bioengineering, and detecting technology. Specifically, the present disclosure discloses use of a set of urine microRNA (miRNA) fingerprints in the diagnosis of human bladder and urothelial carcinoma.

### RELATED ART

Bladder and urothelial carcinoma, for example, bladder cancer (carcinoma of bladder), is a common malignant tumor in the urinary system, of which the incidence rate is apparently higher for men than for women. For the bladder cancer, its incidence rate ranks sixth among male tumors, and its mortality ranks ninth among male tumors.

The vast majority of primary malignant bladder tumors come from epithelial tissues, most of which are urothelial cell carcinoma (UCC), and squamous cell carcinoma and adenocarcinoma are relatively less. At present, the gold standard for clinically diagnosing bladder cancer is urine exfoliation cytology, cystoscopy, and biopsy. The urine exfoliation cytology has high specificity but low sensitivity, and it has been rarely used in clinical test. The cystoscopy is an invasive test, which is so poor on patient compliance that cannot be widely used in screening checkup for bladder cancer. Therefore, it is very necessary to find a simple, economical, non-invasive, and highly sensitive detecting method to be widely used in screening and diagnosing bladder and urothelial carcinoma, monitoring the recurrence of bladder and urothelial carcinoma, and making a prognosis.

Urine samples, the ideal samples for the checkup and screening and early detecting bladder and urothelial carcinoma, are readily available. In recent years, it has been reported that a series of urinary biomarkers (such as matrix protein NMP22) may be helpful in the early diagnosis, prognosis for recurrence, and treatment of bladder and urothelial carcinoma, but have low sensitivity and poor specificity, which cannot meet the needs for early detecting and screening bladder and urothelial carcinoma (for example, refer to Schmitz-Drager B J et al., Considerations on the use of urine markers in the management of patients with low-/intermediate-risk non-muscle invasive bladder cancer. Urol Oncol 2014 October; 32(7):1061-8; Peng Wu et al., New Progress of Epigenetic Biomarkers in Urological Cancer. Disease Markers, Volume 2016, Article ID 9864047, 8 pages Review; and Clark P E et al., NCCN Guidelines Insights: Bladder Cancer, Version 2.2016. J Natl Compr Canc Netw. 2016 October; 14(10):1213-1224).

A micro RNA (miRNA) is a small, single-stranded, and non-coding RNA molecule with a length of about 16-26 nucleotides, which regulates the expression of most of human coding genes. It plays an extremely important role in many life activities such as growth, division, differentiation, development, apoptosis, and disease occurrence (for example, refer to Bartel D P, MicroRNAs: Genomics, Review Biogenesis, Mechanism, and Function. Cell, 2004, Vol. 116, 281-297.).

Therefore, it is necessary to develop a diagnostic kit (especially a miRNA diagnostic kit) for bladder and urothelial carcinoma, especially for the early detection and screening of bladder and urothelial carcinoma, guidance on the treatment and medication of bladder and urothelial carcinoma, and the prognosis assessment. It will have important medical significance and application prospects.

### SUMMARY

According to an aspect, this application provides miRNA combinations, including at least 3 miRNAs selected from a group consisting of: hsa-miR-99a, hsa-miR-141, hsa-miR-151-5p, hsa-miR-96, hsa-miR-125b, hsa-miR-183, hsa-miR-1260, hsa-miR-133a, hsa-miR-429, hsa-miR-143#, hsa-miR-29c#, hsa-miR-29c, hsa-miR-152, hsa-miR-100, hsa-miR-27b, and hsa-miR-497.

According to another aspect, this application provides a method for diagnosing whether a test subject suffers from bladder and urothelial carcinoma or is at a high risk of bladder and urothelial carcinoma, including:

a) obtaining a test urine sample from the test subject;

b) measuring an expression level of each miRNA in the miRNA combination provided by this application in the test urine sample; and

c) assessing whether the test subject suffers from bladder and urothelial carcinoma or is at a high risk of bladder and urothelial carcinoma, based on the expression levels of the miRNAs.

In some embodiments, the miRNA combination includes at least 3, at least 4, at least 5, at least 6, at least 7, at least 8, at least 9, at least 10, at least 11, at least 12, at least 13, at least 14, at least 15, or at least 16 miRNAs selected from a group consisting of: hsa-miR-99a, hsa-miR-141, hsa-miR-151-5p, hsa-miR-96, hsa-miR-125b, hsa-miR-183, hsa-miR-1260, hsa-miR-133a, hsa-miR-429, hsa-miR-143#, hsa-miR-29c#, hsa-miR-29c, hsa-miR-152, hsa-miR-100, hsa-miR-27b, and hsa-miR-497.

In some embodiments, the miRNA combination includes at least 3, at least 4, at least 5, at least 6, at least 7, at least 8, at least 9, at least 10, at least 11, at least 12, at least 13, at least 14, or at least 15 miRNAs selected from a group consisting of: hsa-miR-99a, hsa-miR-141, hsa-miR-151-5p, hsa-miR-96, hsa-miR-125b, hsa-miR-183, hsa-miR-1260, hsa-miR-133a, hsa-miR-429, hsa-miR-143#, hsa-miR-29c#, hsa-miR-29c, hsa-miR-152, hsa-miR-100, and hsa-miR-27b.

In some embodiments, the miRNA combination includes at least 3, at least 4, at least 5, at least 6, at least 7, at least 8, at least 9, at least 10, at least 11, at least 12, at least 13, or at least 14 miRNAs selected from a group consisting of: hsa-miR-99a, hsa-miR-141, hsa-miR-151-5p, hsa-miR-96, hsa-miR-125b, hsa-miR-183, hsa-miR-1260, hsa-miR-133a, hsa-miR-429, hsa-miR-143#, hsa-miR-29c#, hsa-miR-29c, hsa-miR-152, and hsa-miR-100.

In some embodiments, the miRNA combination includes at least 3, at least 4, at least 5, at least 6, at least 7, at least 8, at least 9, at least 10, at least 11, at least 12, or at least 13 miRNAs selected from a group consisting of: hsa-miR-99a (or hsa-miR-100), hsa-miR-141, hsa-miR-151-5p, hsa-miR-96, hsa-miR-125b, hsa-miR-183, hsa-miR-1260, hsa-miR-133a, hsa-miR-429, hsa-miR-143#, hsa-miR-29c#, hsa-miR-29c, and hsa-miR-152.

In some embodiments, the miRNA combination includes at least 3, at least 4, at least 5, at least 6, at least 7, at least 8, at least 9, at least 10, at least 11, or at least 12 miRNAs selected from a group consisting of: hsa-miR-99a (or hsa-miR-100), hsa-miR-141, hsa-miR-151-5p, hsa-miR-96, hsa-miR-125b, hsa-miR-183, hsa-miR-1260, hsa-miR-133a, hsa-miR-429, hsa-miR-143#, hsa-miR-29c#, and hsa-miR-29c.

In some embodiments, the miRNA combination includes at least 3, at least 4, at least 5, at least 6, at least 7, at least 8, at least 9, at least 10, or at least 11 miRNAs selected from a group consisting of: hsa-miR-99a (or hsa-miR-100), hsa-miR-141, hsa-miR-151-5p, hsa-miR-96, hsa-miR-125b, hsa-miR-183, hsa-miR-1260, hsa-miR-133a, hsa-miR-429, hsa-miR-143#, and hsa-miR-29c#.

In some embodiments, the miRNA combination includes at least 3, at least 4, at least 5, at least 6, at least 7, at least 8, at least 9, or at least 10 miRNAs selected from a group consisting of: hsa-miR-99a (or hsa-miR-100), hsa-miR-141, hsa-miR-151-5p, hsa-miR-96, hsa-miR-125b, hsa-miR-183, hsa-miR-1260, hsa-miR-133a, hsa-miR-429, and hsa-miR-143#.

In some embodiments, the miRNA combination includes at least 3, at least 4, at least 5, at least 6, at least 7, at least 8, at least 9, or at least 10 miRNAs selected from a group consisting of: hsa-miR-100, hsa-miR-125b, hsa-miR-133a, hsa-miR-141, hsa-miR-143#, hsa-miR-151-5p, hsa-miR-29c, hsa-miR-29c#, hsa-miR-99a, and hsa-miR-96.

In some embodiments, the miRNA combination includes at least 3, at least 4, at least 5, at least 6, at least 7, at least 8, or at least 9 miRNAs selected from a group consisting of: hsa-miR-99a (or hsa-miR-100), hsa-miR-141, hsa-miR-151-5p, hsa-miR-96, hsa-miR-125b, hsa-miR-183, hsa-miR-1260, hsa-miR-133a, and hsa-miR-429.

In some embodiments, the miRNA combination includes at least 3, at least 4, at least 5, at least 6, at least 7, at least 8, or at least 9 miRNAs selected from a group consisting of: hsa-miR-100, hsa-miR-125b, hsa-miR-133a, hsa-miR-141, hsa-miR-143#, hsa-miR-151-5p, hsa-miR-29c, hsa-miR-99a, and hsa-miR-29c#.

In some embodiments, the miRNA combination includes at least 3, at least 4, at least 5, at least 6, at least 7, at least 8, or at least 9 miRNAs selected from a group consisting of: hsa-miR-100, hsa-miR-125b, hsa-miR-133a, hsa-miR-141, hsa-miR-143#, hsa-miR-151-5p, hsa-miR-29c, hsa-miR-99a, and hsa-miR-96.

In some embodiments, the miRNA combination includes at least 3, at least 4, at least 5, at least 6, at least 7, or at least 8 miRNAs selected from a group consisting of: hsa-miR-99a (or hsa-miR-100), hsa-miR-141, hsa-miR-151-5p, hsa-miR-96, hsa-miR-125b, hsa-miR-183, hsa-miR-1260, and hsa-miR-133a.

In some embodiments, the miRNA combination includes at least 3, at least 4, at least 5, at least 6, at least 7, or at least 8 miRNAs selected from a group consisting of: hsa-miR-99a, hsa-miR-141, hsa-miR-151-5p, hsa-miR-143#, hsa-miR-125b, hsa-miR-29c, hsa-miR-100, and hsa-miR-133a.

In some embodiments, the miRNA combination includes at least 3, at least 4, at least 5, at least 6, or at least 7 miRNAs selected from a group consisting of: hsa-miR-99a (or hsa-miR-100), hsa-miR-141, hsa-miR-151-5p, hsa-miR-96, hsa-miR-125b, hsa-miR-183, and hsa-miR-1260.

In some embodiments, the miRNA combination includes at least 3, at least 4, at least 5, at least 6, or at least 7 miRNAs selected from a group consisting of: hsa-miR-141, hsa-miR-151-5p, hsa-miR-143#, hsa-miR-125b, hsa-miR-29c, hsa-miR-100 (or hsa-miR-99a), and hsa-miR-133a.

In some embodiments, the miRNA combination includes the following 7 miRNAs: hsa-miR-99a, hsa-miR-141, hsa-miR-151-5p, hsa-miR-125b, hsa-miR-133a, hsa-miR-143#, and hsa-miR-29c; or includes the following 7 miRNAs: hsa-miR-100, hsa-miR-141, hsa-miR-151-5p, hsa-miR-125b, hsa-miR-133a, hsa-miR-143#, and hsa-miR-29c.

In some embodiments, the miRNA combination includes at least 3, at least 4, at least 5, or at least 6 miRNAs selected from a group consisting of: hsa-miR-99a (or hsa-miR-100), hsa-miR-141, hsa-miR-151-5p, hsa-miR-96, hsa-miR-125b, and hsa-miR-183.

In some embodiments, the miRNA combination includes at least 3, at least 4, or at least 5 miRNAs selected from a group consisting of: hsa-miR-99a (or hsa-miR-100), hsa-miR-141, hsa-miR-151-5p, hsa-miR-96, and hsa-miR-125b.

In some embodiments, the miRNA combination includes the following 4 miRNAs: hsa-miR-99a, hsa-miR-141, hsa-miR-151-5p, and hsa-miR-96; or includes the following 4 miRNAs: hsa-miR-100, hsa-miR-141, hsa-miR-151-5p, and hsa-miR-96.

In some embodiments, the miRNA combination includes at least 3, or at least 4 miRNAs selected from a group consisting of: hsa-miR-99a (or hsa-miR-100), hsa-miR-141, hsa-miR-151-5p, and hsa-miR-96.

In some embodiments, the miRNA combination includes the following miRNA combination: hsa-miR-99a, hsa-miR-141, and hsa-miR-151-5p; or includes the following miRNA combination: hsa-miR-100, hsa-miR-141, and hsa-miR-151-5p.

In some embodiments, the miRNA combination further includes hsa-miR-96.

In some embodiments, the miRNA combination further includes hsa-miR-125b.

In some embodiments, the miRNA combination further includes hsa-miR-183.

In some embodiments, the miRNA combination further includes hsa-miR-1260.

In some embodiments, the miRNA combination further includes hsa-miR-133a.

In some embodiments, the miRNA combination further includes hsa-miR-429.

In some embodiments, the miRNA combination further includes hsa-miR-143#.

In some embodiments, the miRNA combination further includes hsa-miR-29c#.

In some embodiments, the miRNA combination further includes hsa-miR-29c.

In some embodiments, the miRNA combination further includes hsa-miR-152.

In some embodiments, the miRNA combination further includes hsa-miR-100.

In some embodiments, the miRNA combination further includes hsa-miR-27b.

In some embodiments, the miRNA combination further includes hsa-miR-497.

In some embodiments, the miRNA combination includes any one of miRNA combinations selected from combination 1, combination 2, combination 3, combination 4, combination 5, combination 6, combination 7, combination 8, combination 9, combination 10, combination 11, combination 12, combination 13, combination 14, combination 15, combination 16, combination 17, combination 18, combination 19, combination 20, combination 21, and combination 22, where:

1) the combination 1 includes: hsa-miR-99a, hsa-miR-141, hsa-miR-151-5p, hsa-miR-96, hsa-miR-125b, hsa-miR-183, hsa-miR-1260, hsa-miR-143#, hsa-miR-29c#, hsa-miR-152, and hsa-miR-100;

2) the combination 2 includes: hsa-miR-99a, hsa-miR-141, hsa-miR-151-5p, hsa-miR-96, hsa-miR-125b, hsa-miR-183, hsa-miR-1260, hsa-miR-143#, hsa-miR-29c#, hsa-miR-29c, hsa-miR-152, and hsa-miR-100;

3) the combination 3 includes: hsa-miR-99a, hsa-miR-141, hsa-miR-151-5p, hsa-miR-1260, hsa-miR-133a, hsa-miR-429, hsa-miR-143#, hsa-miR-29c#, hsa-miR-29c, and hsa-miR-152; or includes: hsa-miR-100, hsa-miR-141, hsa-miR-151-5p, hsa-miR-1260, hsa-miR-133a, hsa-miR-429, hsa-miR-143#, hsa-miR-29c#, hsa-miR-29c, and hsa-miR-152;

4) the combination 4 includes: hsa-miR-99a, hsa-miR-96, hsa-miR-125b, hsa-miR-183, hsa-miR-429, hsa-miR-143#, hsa-miR-29c#, and hsa-miR-27b; or includes: hsa-miR-100, hsa-miR-96, hsa-miR-125b, hsa-miR-183, hsa-miR-429, hsa-miR-143#, hsa-miR-29c#, and hsa-miR-27b;

5) the combination 5 includes: hsa-miR-99a, hsa-miR-96, hsa-miR-125b, hsa-miR-183, hsa-miR-429, hsa-miR-143#, hsa-miR-29c#, and hsa-miR-152; or includes: hsa-miR-100, hsa-miR-96, hsa-miR-125b, hsa-miR-183, hsa-miR-429, hsa-miR-143#, hsa-miR-29c#, and hsa-miR-152;

6) the combination 6 includes: hsa-miR-99a, hsa-miR-141, hsa-miR-151-5p, hsa-miR-96, hsa-miR-183, hsa-miR-1260, hsa-miR-133a, hsa-miR-143#, hsa-miR-29c, and hsa-miR-27b; or includes: hsa-miR-100, hsa-miR-141, hsa-miR-151-5p, hsa-miR-96, hsa-miR-183, hsa-miR-1260, hsa-miR-133a, hsa-miR-143#, hsa-miR-29c, and hsa-miR-27b;

7) the combination 7 includes: hsa-miR-99a, hsa-miR-141, hsa-miR-151-5p, hsa-miR-1260, hsa-miR-429, hsa-miR-143#, hsa-miR-29c#, hsa-miR-29c, and hsa-miR-152; or includes: hsa-miR-100, hsa-miR-141, hsa-miR-151-5p, hsa-miR-1260, hsa-miR-429, hsa-miR-143#, hsa-miR-29c#, hsa-miR-29c, and hsa-miR-152;

8) the combination 8 includes: hsa-miR-99a, hsa-miR-141, hsa-miR-151-5p, hsa-miR-96, hsa-miR-125b, hsa-miR-1260, hsa-miR-133a, hsa-miR-429, hsa-miR-143#, hsa-miR-29c#, hsa-miR-29c, and hsa-miR-152; or includes: hsa-miR-100, hsa-miR-141, hsa-miR-151-5p, hsa-miR-96, hsa-miR-125b, hsa-miR-1260, hsa-miR-133a, hsa-miR-429, hsa-miR-143#, hsa-miR-29c#, hsa-miR-29c, and hsa-miR-152;

9) the combination 9 includes: hsa-miR-99a, hsa-miR-141, hsa-miR-151-5p, hsa-miR-96, hsa-miR-125b, hsa-miR-183, hsa-miR-1260, hsa-miR-143#, hsa-miR-29c#, hsa-miR-29c, hsa-miR-152, and hsa-miR-100;

10) the combination 10 includes: hsa-miR-99a, hsa-miR-141, hsa-miR-151-5p, hsa-miR-96, hsa-miR-125b, hsa-miR-183, hsa-miR-1260, hsa-miR-133a, hsa-miR-143#, hsa-miR-29c#, hsa-miR-29c, hsa-miR-152, and hsa-miR-27b; or includes: hsa-miR-100, hsa-miR-141, hsa-miR-151-5p, hsa-miR-96, hsa-miR-125b, hsa-miR-183, hsa-miR-1260, hsa-miR-133a, hsa-miR-143#, hsa-miR-29c#, hsa-miR-29c, hsa-miR-152, and hsa-miR-27b;

11) the combination 11 includes: hsa-miR-99a, hsa-miR-141, hsa-miR-151-5p, hsa-miR-96, hsa-miR-125b, hsa-miR-183, hsa-miR-1260, hsa-miR-133a, and hsa-miR-429; or includes: hsa-miR-100, hsa-miR-141, hsa-miR-151-5p, hsa-miR-96, hsa-miR-125b, hsa-miR-183, hsa-miR-1260, hsa-miR-133a, and hsa-miR-429;

12) the combination 12 includes: hsa-miR-99a, hsa-miR-141, hsa-miR-151-5p, hsa-miR-96, hsa-miR-125b, hsa-miR-183, and hsa-miR-133a; or includes: hsa-miR-100, hsa-miR-141, hsa-miR-151-5p, hsa-miR-96, hsa-miR-125b, hsa-miR-183, and hsa-miR-133a;

13) the combination 13 includes: hsa-miR-99a, hsa-miR-141, hsa-miR-151-5p, hsa-miR-96, hsa-miR-125b, hsa-miR-183, hsa-miR-1260, hsa-miR-133a, hsa-miR-429, and hsa-miR-29c; or includes: hsa-miR-100, hsa-miR-141, hsa-miR-151-5p, hsa-miR-96, hsa-miR-125b, hsa-miR-183, hsa-miR-1260, hsa-miR-133a, hsa-miR-429, and hsa-miR-29c;

14) the combination 14 includes: hsa-miR-99a, hsa-miR-141, hsa-miR-151-5p, hsa-miR-96, hsa-miR-125b, hsa-miR-183, hsa-miR-1260, hsa-miR-133a, hsa-miR-429, and hsa-miR-497; or includes: hsa-miR-100, hsa-miR-141, hsa-miR-151-5p, hsa-miR-96, hsa-miR-125b, hsa-miR-183, hsa-miR-1260, hsa-miR-133a, hsa-miR-429, and hsa-miR-497;

15) the combination 15 includes: hsa-miR-99a, hsa-miR-141, hsa-miR-151-5p, hsa-miR-96, hsa-miR-125b, hsa-miR-183, hsa-miR-1260, hsa-miR-133a, hsa-miR-429, hsa-miR-143#, and hsa-miR-29c; or includes: hsa-miR-100, hsa-miR-141, hsa-miR-151-5p, hsa-miR-96, hsa-miR-125b, hsa-miR-183, hsa-miR-1260, hsa-miR-133a, hsa-miR-429, hsa-miR-143#, and hsa-miR-29c;

16) the combination 16 includes: hsa-miR-99a, hsa-miR-141, hsa-miR-151-5p, hsa-miR-96, hsa-miR-125b, hsa-miR-133a, hsa-miR-143#, and hsa-miR-29c#; or includes: hsa-miR-100, hsa-miR-141, hsa-miR-151-5p, hsa-miR-96, hsa-miR-125b, hsa-miR-133a, hsa-miR-143#, and hsa-miR-29c#;

17) the combination 17 includes: hsa-miR-99a, hsa-miR-141, hsa-miR-151-5p, hsa-miR-96, hsa-miR-125b, hsa-miR-183, hsa-miR-133a, hsa-miR-429, hsa-miR-143#, and hsa-miR-29c#; or includes: hsa-miR-100, hsa-miR-141, hsa-miR-151-5p, hsa-miR-96, hsa-miR-125b, hsa-miR-183, hsa-miR-133a, hsa-miR-429, hsa-miR-143#, and hsa-miR-29c#;

18) the combination 18 includes: hsa-miR-100, hsa-miR-125b, hsa-miR-133a, hsa-miR-141, hsa-miR-143#, hsa-miR-151-5p, hsa-miR-29c, hsa-miR-29c#, hsa-miR-99a, and hsa-miR-96;

19) the combination 19 includes: hsa-miR-100, hsa-miR-125b, hsa-miR-133a, hsa-miR-141, hsa-miR-143#, hsa-miR-151-5p, hsa-miR-29c, and hsa-miR-99a;

20) the combination 20 includes: hsa-miR-100, hsa-miR-125b, hsa-miR-133a, hsa-miR-141, hsa-miR-143#, hsa-miR-151-5p, hsa-miR-29c, hsa-miR-99a, and hsa-miR-29c#;

21) the combination 21 includes: hsa-miR-100, hsa-miR-125b, hsa-miR-133a, hsa-miR-141, hsa-miR-143#, hsa-miR-151-5p, hsa-miR-29c, hsa-miR-99a, and hsa-miR-96; and

22) the combination 22 includes: hsa-miR-141, hsa-miR-151-5p, hsa-miR-143#, hsa-miR-125b, hsa-miR-29c, hsa-miR-100, and hsa-miR-133a; or includes: hsa-miR-141, hsa-miR-151-5p, hsa-miR-143#, hsa-miR-125b, hsa-miR-29c, hsa-miR-99a, and hsa-miR-133a.

In some embodiments, the expression levels of the miRNAs is normalized to an endogenous reference.

In some embodiments, the endogenous reference includes one or more miRNAs from the miRNA combination.

In some embodiments, the endogenous reference includes hsa-miR-99a or hsa-miR-100.

In some embodiments, the expression level of each miRNA is measured by using a primer, a probe, or an intercalator. The primer or the probe has a hybridization region, and the hybridization region can hybridize with the miRNA or the complement sequence thereof. The intercalator can generate a signal when inserted into a DNA double strand.

In some embodiments, the hybridization region is at least 60% complementary or substantially complementary to the nucleotide sequence of the miRNA or the complement sequence thereof.

In some embodiments, before the expression level of each miRNA is measured, the method further includes reverse transcription of each miRNA into cDNA.

In some embodiments, the expression levels of the miRNAs are measured by an amplification-based method, a hybridization-based method, and/or a sequencing-based method.

In some embodiments, before the expression levels of the miRNAs are measured, the method further includes enriching RNAs from the test urine sample.

In some embodiments, the enrichment includes extraction of RNAs from the precipitate obtained by centrifugating the test urine sample.

In some embodiments, the step c) further includes calculating the expression pattern of the miRNA combination, based on the expression levels of the miRNAs.

In some embodiments, the expression pattern is calculated through a function or model related to the expression level of each miRNA and a decision weight of each miRNA for sample states, and the function or model is calculated by a classification algorithm.

In some embodiments, the classification algorithm is selected from support vector machines, linear discriminant analysis, logistic regression, naive Bayes classification, perceptron classification, quadratic classification, k-nearest neighbors, boosting, decision tree, random forest, neural network, and learning vector quantization.

In some embodiments, the classification algorithm is support vector machines.

In some embodiments, the classification algorithm is subjected to at least one training of a positive training data set and a negative training data set to provide one or more decision weights of the function or model for calculating the expression pattern, the positive training data set includes the expression level of each miRNA in the miRNA combination from a plurality of urine samples of subjects with bladder and urothelial carcinoma, and the negative training data set includes the expression level of each miRNA in the miRNA combination from a plurality of urine samples of subjects without bladder and urothelial carcinoma.

In some embodiments, the training includes training with the positive training data set and the negative training data set to provide one or more decision weights of the function or model for calculating a positive expression pattern and one or more decision weights of the function or model for calculating a negative expression pattern.

In some embodiments, the expression pattern is a score between 0 and 1.

In some embodiments, a threshold is determined based on a score of the positive expression pattern and a score of the negative expression pattern, wherein the threshold is able to distinguish the positive expression pattern from the negative expression pattern.

In some embodiments, the method further includes comparing a score of the expression pattern calculated based on the expression levels of the miRNAs from the test urine sample with the threshold to assess whether the test subject suffers from bladder and urothelial carcinoma or is at a high risk of bladder and urothelial carcinoma.

In some embodiments, the threshold is a value between 0.2 and 0.8, and if the score of the expression pattern is greater than the threshold, the test subject is assessed as suffering from bladder and urothelial carcinoma or being at a high risk of bladder and urothelial carcinoma.

In some embodiments, the threshold is 0.4.

In some embodiments, the method further includes administering a bladder and urothelial carcinoma therapy to the test subject assessed as suffering from bladder and urothelial carcinoma or being at a high risk of bladder and urothelial carcinoma in the step c).

In some embodiments, the bladder and urothelial carcinoma therapy includes chemotherapy, radiotherapy, immunotherapy, surgery, or anti-cancer drug therapy.

In some embodiments, the bladder and urothelial carcinoma includes bladder cancer, renal pelvic carcinoma, ureter cancer, and urinary outflow tract cancer.

According to another aspect, this application provides a set of isolated oligonucleotides including a hybridization region, where the hybridization region in each of the oligonucleotides can hybridize with a corresponding miRNA in a miRNA combination provided by this application or a complement sequence thereof, and the miRNA combination includes:

1) at least 3, at least 4, at least 5, at least 6, at least 7, at least 8, at least 9, at least 10, at least 11, at least 12, at least 13, at least 14, at least 15, or at least 16 miRNAs selected from a group consisting of: hsa-miR-99a, hsa-miR-141, hsa-miR-151-5p, hsa-miR-96, hsa-miR-125b, hsa-miR-183, hsa-miR-1260, hsa-miR-133a, hsa-miR-429, hsa-miR-143#, hsa-miR-29c#, hsa-miR-29c, hsa-miR-152, hsa-miR-100, hsa-miR-27b, and hsa-miR-497;

2) at least 3, at least 4, at least 5, at least 6, at least 7, at least 8, at least 9, at least 10, at least 11, at least 12, at least 13, at least 14, or at least 15 miRNAs selected from a group consisting of: hsa-miR-99a, hsa-miR-141, hsa-miR-151-5p, hsa-miR-96, hsa-miR-125b, hsa-miR-183, hsa-miR-1260, hsa-miR-133a, hsa-miR-429, hsa-miR-143#, hsa-miR-29c#, hsa-miR-29c, hsa-miR-152, hsa-miR-100, and hsa-miR-27b;

3) at least 3, at least 4, at least 5, at least 6, at least 7, at least 8, at least 9, at least 10, at least 11, at least 12, at least 13, or at least 14 miRNAs selected from a group consisting of: hsa-miR-99a, hsa-miR-141, hsa-miR-151-5p, hsa-miR-96, hsa-miR-125b, hsa-miR-183, hsa-miR-1260, hsa-miR-133a, hsa-miR-429, hsa-miR-143#, hsa-miR-29c#, hsa-miR-29c, hsa-miR-152, and hsa-miR-100;

4) at least 3, at least 4, at least 5, at least 6, at least 7, at least 8, at least 9, at least 10, at least 11, at least 12, or at least 13 miRNAs selected from a group consisting of: hsa-miR-99a (or hsa-miR-100), hsa-miR-141, hsa-miR-151-5p, hsa-miR-96, hsa-miR-125b, hsa-miR-183, hsa-miR-1260, hsa-miR-133a, hsa-miR-429, hsa-miR-143#, hsa-miR-29c#, hsa-miR-29c, and hsa-miR-152;

5) at least 3, at least 4, at least 5, at least 6, at least 7, at least 8, at least 9, at least 10, at least 11, or at least 12 miRNAs selected from a group consisting of: hsa-miR-99a (or hsa-miR-100), hsa-miR-141, hsa-miR-151-5p, hsa-miR-96, hsa-miR-125b, hsa-miR-183, hsa-miR-1260, hsa-miR-133a, hsa-miR-429, hsa-miR-143#, hsa-miR-29c#, and hsa-miR-29c;

6) at least 3, at least 4, at least 5, at least 6, at least 7, at least 8, at least 9, at least 10, or at least 11 miRNAs selected from a group consisting of: hsa-miR-99a (or hsa-miR-100), hsa-miR-141, hsa-miR-151-5p, hsa-miR-96, hsa-miR-125b, hsa-miR-183, hsa-miR-1260, hsa-miR-133a, hsa-miR-429, hsa-miR-143#, and hsa-miR-29c#;

7) at least 3, at least 4, at least 5, at least 6, at least 7, at least 8, at least 9, or at least 10 miRNAs selected from a group consisting of: hsa-miR-99a (or hsa-miR-100), hsa-miR-141, hsa-miR-151-5p, hsa-miR-96, hsa-miR-125b, hsa-miR-183, hsa-miR-1260, hsa-miR-133a, hsa-miR-429, and hsa-miR-143#;

8) at least 3, at least 4, at least 5, at least 6, at least 7, at least 8, at least 9, or at least 10 miRNAs selected from a group consisting of: hsa-miR-100, hsa-miR-125b, hsa-miR-133a, hsa-miR-141, hsa-miR-143#, hsa-miR-151-5p, hsa-miR-29c, hsa-miR-29c#, hsa-miR-99a, and hsa-miR-96;

9) at least 3, at least 4, at least 5, at least 6, at least 7, at least 8, or at least 9 miRNAs selected from a group consisting of: hsa-miR-99a (or hsa-miR-100), hsa-miR-141, hsa-miR-151-5p, hsa-miR-96, hsa-miR-125b, hsa-miR-183, hsa-miR-1260, hsa-miR-133a, and hsa-miR-429;

10) at least 3, at least 4, at least 5, at least 6, at least 7, at least 8, or at least 9 miRNAs selected from a group consisting of: hsa-miR-100, hsa-miR-125b, hsa-miR-133a, hsa-miR-141, hsa-miR-143#, hsa-miR-151-5p, hsa-miR-29c, hsa-miR-99a, and hsa-miR-29c#;

11) at least 3, at least 4, at least 5, at least 6, at least 7, at least 8, or at least 9 miRNAs selected from a group consisting of: hsa-miR-100, hsa-miR-125b, hsa-miR-133a, hsa-miR-141, hsa-miR-143#, hsa-miR-151-5p, hsa-miR-29c, hsa-miR-99a, and hsa-miR-96;

12) at least 3, at least 4, at least 5, at least 6, at least 7, or at least 8 miRNAs selected from a group consisting of: hsa-miR-99a (or hsa-miR-100), hsa-miR-141, hsa-miR-151-5p, hsa-miR-96, hsa-miR-125b, hsa-miR-183, hsa-miR-1260, and hsa-miR-133a;

13) at least 3, at least 4, at least 5, at least 6, at least 7, or at least 8 miRNAs selected from a group consisting of: hsa-miR-99a, hsa-miR-141, hsa-miR-151-5p, hsa-miR-143#, hsa-miR-125b, hsa-miR-29c, hsa-miR-100, and hsa-miR-133a;

14) at least 3, at least 4, at least 5, at least 6, or at least 7 miRNAs selected from a group consisting of: hsa-miR-99a (or hsa-miR-100), hsa-miR-141, hsa-miR-151-5p, hsa-miR-96, hsa-miR-125b, hsa-miR-183, and hsa-miR-1260;

15) at least 3, at least 4, at least 5, at least 6, or at least 7 miRNAs selected from a group consisting of: hsa-miR-141, hsa-miR-151-5p, hsa-miR-143#, hsa-miR-125b, hsa-miR-29c, hsa-miR-100 (or hsa-miR-99a), and hsa-miR-133a;

16) at least 3, at least 4, at least 5, or at least 6 miRNAs selected from a group consisting of: hsa-miR-99a (or hsa-miR-100), hsa-miR-141, hsa-miR-151-5p, hsa-miR-96, hsa-miR-125b, and hsa-miR-183;

17) at least 3, at least 4, or at least 5 miRNAs selected from a group consisting of: hsa-miR-99a (or hsa-miR-100), hsa-miR-141, hsa-miR-151-5p, hsa-miR-96, and hsa-miR-125b; or

18) at least 3, or at least 4 miRNAs selected from a group consisting of: hsa-miR-99a (or hsa-miR-100), hsa-miR-141, hsa-miR-151-5p, and hsa-miR-96.

In some embodiments, the miRNA combination includes the following miRNA combination: hsa-miR-99a, hsa-miR-141, and hsa-miR-151-5p; or includes the following miRNA combination: hsa-miR-100, hsa-miR-141, and hsa-miR-151-5p.

In some embodiments, the miRNA combination further includes hsa-miR-96.

In some embodiments, the miRNA combination further includes hsa-miR-125b.

In some embodiments, the miRNA combination further includes hsa-miR-183.

In some embodiments, the miRNA combination further includes hsa-miR-1260.

In some embodiments, the miRNA combination further includes hsa-miR-133a.

In some embodiments, the miRNA combination further includes hsa-miR-429.

In some embodiments, the miRNA combination further includes hsa-miR-143#.

In some embodiments, the miRNA combination further includes hsa-miR-29c#.

In some embodiments, the miRNA combination further includes hsa-miR-29c.

In some embodiments, the miRNA combination further includes hsa-miR-152.

In some embodiments, the miRNA combination further includes hsa-miR-100.

In some embodiments, the miRNA combination further includes hsa-miR-29b.

In some embodiments, the miRNA combination further includes hsa-miR-497.

In some embodiments, the miRNA combination includes hsa-miR-99a, hsa-miR-141, hsa-miR-151-5p, hsa-miR-96, hsa-miR-125b, hsa-miR-183, and hsa-miR-133a.

In some embodiments, the miRNA combination includes hsa-miR-99a, hsa-miR-141, hsa-miR-151-5p, hsa-miR-96, hsa-miR-125b, hsa-miR-183, hsa-miR-1260, hsa-miR-133a, and hsa-miR-429.

In some embodiments, the miRNA combination includes any one of miRNA combinations selected from combination 1, combination 2, combination 3, combination 4, combination 5, combination 6, combination 7, combination 8, combination 9, combination 10, combination 11, combination 12, combination 13, combination 14, combination 15, combination 16, combination 17, combination 18, combination 19, combination 20, combination 21, and combination 22, where:

1) the combination 1 includes: hsa-miR-99a, hsa-miR-141, hsa-miR-151-5p, hsa-miR-96, hsa-miR-125b, hsa-miR-183, hsa-miR-1260, hsa-miR-143#, hsa-miR-29c#, hsa-miR-152, and hsa-miR-100;

2) the combination 2 includes: hsa-miR-99a, hsa-miR-141, hsa-miR-151-5p, hsa-miR-96, hsa-miR-125b, hsa-miR-183, hsa-miR-1260, hsa-miR-143#, hsa-miR-29c#, hsa-miR-29c, hsa-miR-152, and hsa-miR-100;

3) the combination 3 includes: hsa-miR-99a, hsa-miR-141, hsa-miR-151-5p, hsa-miR-1260, hsa-miR-133a, hsa-miR-429, hsa-miR-143#, hsa-miR-29c#, hsa-miR-29c, and hsa-miR-152; or includes: hsa-miR-100, hsa-miR-141, hsa-miR-151-5p, hsa-miR-1260, hsa-miR-133a, hsa-miR-429, hsa-miR-143#, hsa-miR-29c#, hsa-miR-29c, and hsa-miR-152;

4) the combination 4 includes: hsa-miR-99a, hsa-miR-96, hsa-miR-125b, hsa-miR-183, hsa-miR-429, hsa-miR-143#, hsa-miR-29c#, and hsa-miR-27b; or includes: hsa-miR-100, hsa-miR-96, hsa-miR-125b, hsa-miR-183, hsa-miR-429, hsa-miR-143#, hsa-miR-29c#, and hsa-miR-27b;

5) the combination 5 includes: hsa-miR-99a, hsa-miR-96, hsa-miR-125b, hsa-miR-183, hsa-miR-429, hsa-miR-143#, hsa-miR-29c#, and hsa-miR-152; or includes: hsa-miR-100, hsa-miR-96, hsa-miR-125b, hsa-miR-183, hsa-miR-429, hsa-miR-143#, hsa-miR-29c#, and hsa-miR-152;

6) the combination 6 includes: hsa-miR-99a, hsa-miR-141, hsa-miR-151-5p, hsa-miR-96, hsa-miR-183, hsa-miR-1260, hsa-miR-133a, hsa-miR-143#, hsa-miR-29c, and hsa-miR-27b; or includes: hsa-miR-100, hsa-miR-141, hsa-miR-151-5p, hsa-miR-96, hsa-miR-183, hsa-miR-1260, hsa-miR-133a, hsa-miR-143#, hsa-miR-29c, and hsa-miR-27b;

7) the combination 7 includes: hsa-miR-99a, hsa-miR-141, hsa-miR-151-5p, hsa-miR-1260, hsa-miR-429, hsa-miR-143#, hsa-miR-29c#, hsa-miR-29c, and hsa-miR-152; or includes: hsa-miR-100, hsa-miR-141, hsa-miR-151-5p, hsa-miR-1260, hsa-miR-429, hsa-miR-143#, hsa-miR-29c#, hsa-miR-29c, and hsa-miR-152;

8) the combination 8 includes: hsa-miR-99a, hsa-miR-141, hsa-miR-151-5p, hsa-miR-96, hsa-miR-125b, hsa-miR-1260, hsa-miR-133a, hsa-miR-429, hsa-miR-143#, hsa-miR-29c#, hsa-miR-29c, and hsa-miR-152; or includes: hsa-miR-100, hsa-miR-141, hsa-miR-151-5p, hsa-miR-96, hsa-miR-125b, hsa-miR-1260, hsa-miR-133a, hsa-miR-429, hsa-miR-143#, hsa-miR-29c#, hsa-miR-29c, and hsa-miR-152;

9) the combination 9 includes: hsa-miR-99a, hsa-miR-141, hsa-miR-151-5p, hsa-miR-96, hsa-miR-125b, hsa-miR-183, hsa-miR-1260, hsa-miR-143#, hsa-miR-29c#, hsa-miR-29c, hsa-miR-152, and hsa-miR-100;

10) the combination 10 includes: hsa-miR-99a, hsa-miR-141, hsa-miR-151-5p, hsa-miR-96, hsa-miR-125b, hsa-miR-183, hsa-miR-1260, hsa-miR-133a, hsa-miR-143#, hsa-miR-29c#, hsa-miR-29c, hsa-miR-152, and hsa-miR-27b; or includes: hsa-miR-100, hsa-miR-141, hsa-miR-151-5p, hsa-miR-96, hsa-miR-125b, hsa-miR-183, hsa-miR-1260, hsa-miR-133a, hsa-miR-143#, hsa-miR-29c#, hsa-miR-29c, hsa-miR-152, and hsa-miR-27b;

11) the combination 11 includes: hsa-miR-99a, hsa-miR-141, hsa-miR-151-5p, hsa-miR-96, hsa-miR-125b, hsa-miR-183, hsa-miR-1260, hsa-miR-133a, and hsa-miR-429; or includes: hsa-miR-100, hsa-miR-141, hsa-miR-151-5p, hsa-miR-96, hsa-miR-125b, hsa-miR-183, hsa-miR-1260, hsa-miR-133a, and hsa-miR-429;

12) the combination 12 includes: hsa-miR-99a, hsa-miR-141, hsa-miR-151-5p, hsa-miR-96, hsa-miR-125b, hsa-miR-183, and hsa-miR-133a; or includes: hsa-miR-100, hsa-miR-141, hsa-miR-151-5p, hsa-miR-96, hsa-miR-125b, hsa-miR-183, and hsa-miR-133a;

13) the combination 13 includes: hsa-miR-99a, hsa-miR-141, hsa-miR-151-5p, hsa-miR-96, hsa-miR-125b, hsa-miR-183, hsa-miR-1260, hsa-miR-133a, hsa-miR-429, and hsa-miR-29c; or includes: hsa-miR-100, hsa-miR-141, hsa-miR-151-5p, hsa-miR-96, hsa-miR-125b, hsa-miR-183, hsa-miR-1260, hsa-miR-133a, hsa-miR-429, and hsa-miR-29c;

14) the combination 14 includes: hsa-miR-99a, hsa-miR-141, hsa-miR-151-5p, hsa-miR-96, hsa-miR-125b, hsa-miR-183, hsa-miR-1260, hsa-miR-133a, hsa-miR-429, and hsa-miR-497; or includes: hsa-miR-100, hsa-miR-141, hsa-miR-151-5p, hsa-miR-96, hsa-miR-125b, hsa-miR-183, hsa-miR-1260, hsa-miR-133a, hsa-miR-429, and hsa-miR-497;

15) the combination 15 includes: hsa-miR-99a, hsa-miR-141, hsa-miR-151-5p, hsa-miR-96, hsa-miR-125b, hsa-miR-183, hsa-miR-1260, hsa-miR-133a, hsa-miR-429, hsa-miR-143#, and hsa-miR-29c; or includes: hsa-miR-100, hsa-miR-141, hsa-miR-151-5p, hsa-miR-96, hsa-miR-125b, hsa-miR-183, hsa-miR-1260, hsa-miR-133a, hsa-miR-429, hsa-miR-143#, and hsa-miR-29c;

16) the combination 16 includes: hsa-miR-99a, hsa-miR-141, hsa-miR-151-5p, hsa-miR-96, hsa-miR-125b, hsa-miR-133a, hsa-miR-143#, and hsa-miR-29c#; or includes: hsa-miR-100, hsa-miR-141, hsa-miR-151-5p, hsa-miR-96, hsa-miR-125b, hsa-miR-133a, hsa-miR-143#, and hsa-miR-29c#; or

17) the combination 17 includes: hsa-miR-99a, hsa-miR-141, hsa-miR-151-5p, hsa-miR-96, hsa-miR-125b, hsa-miR-183, hsa-miR-133a, hsa-miR-429, hsa-miR-143#, and hsa-miR-29c#; or includes: hsa-miR-100, hsa-miR-141, hsa-miR-151-5p, hsa-miR-96, hsa-miR-125b, hsa-miR-183, hsa-miR-133a, hsa-miR-429, hsa-miR-143#, and hsa-miR-29c#;

18) the combination 18 includes: hsa-miR-100, hsa-miR-125b, hsa-miR-133a, hsa-miR-141, hsa-miR-143#, hsa-miR-151-5p, hsa-miR-29c, hsa-miR-29c#, hsa-miR-99a, and hsa-miR-96;

19) the combination 19 includes: hsa-miR-100, hsa-miR-125b, hsa-miR-133a, hsa-miR-141, hsa-miR-143#, hsa-miR-151-5p, hsa-miR-29c, and hsa-miR-99a;

20) the combination 20 includes: hsa-miR-100, hsa-miR-125b, hsa-miR-133a, hsa-miR-141, hsa-miR-143#, hsa-miR-151-5p, hsa-miR-29c, hsa-miR-99a, and hsa-miR-29c#;

21) the combination 21 includes: hsa-miR-100, hsa-miR-125b, hsa-miR-133a, hsa-miR-141, hsa-miR-143#, hsa-miR-151-5p, hsa-miR-29c, hsa-miR-99a, and hsa-miR-96; and

22) the combination 22 includes: hsa-miR-141, hsa-miR-151-5p, hsa-miR-143#, hsa-miR-125b, hsa-miR-29c, hsa-miR-100, and hsa-miR-133a; or includes: hsa-miR-141, hsa-miR-151-5p, hsa-miR-143#, hsa-miR-125b, hsa-miR-29c, hsa-miR-99a, and hsa-miR-133a.

In some embodiments, the miRNA combination further includes a reference oligonucleotide that can specifically bind to an endogenous reference nucleic acid.

In some embodiments, the hybridization region is complementary or substantially complementary to the nucleotide sequence of the corresponding miRNA or a complement sequence thereof.

In some embodiments, the substantial complementation refers to including no more than 1, 2, or 3 base mispairings.

In some embodiments, the oligonucleotide includes an oligonucleotide primer.

In some embodiments, the oligonucleotide includes an oligonucleotide probe.

In some embodiments, the oligonucleotide primer or the oligonucleotide probe further includes a detectable marker.

In some embodiments, the detectable marker includes: a chromophore, an isotopic label, a heavy metal, a fluorophore, a chemiluminescent group, a visible or fluorescent particle, a nucleic acid, a binding ligand, or a catalyst (such as an enzyme).

In some embodiments, the primer or the probe includes a fluorophore and further includes a quencher.

In some embodiments, the oligonucleotides are immobilized on a solid support.

According to another aspect, this application further provides use of a kit prepared with the isolated oligonucleotides provided by this application for detecting the miRNA combination.

According to another aspect, this application further provides use of a kit prepared with the isolated oligonucleotides provided by this application for diagnosing whether a test subject suffers from bladder and urothelial carcinoma or is at a high risk of bladder and urothelial carcinoma.

According to another aspect, this application provides a miRNA detecting chip, including: the isolated oligonucleotides provided by this application immobilized on a solid support.

In some embodiments, the isolated oligonucleotides are immobilized in a way that they are spatially isolated from each other.

In some embodiments, the isolated oligonucleotide includes an oligonucleotide primer or an oligonucleotide probe.

In some embodiments, the oligonucleotide probe further includes a detectable marker.

In some embodiments, the marker includes: a chromophore, an isotopic label, a heavy metal, a fluorophore, a chemiluminescent group, a visible or fluorescent particle, a nucleic acid, a binding ligand, or a catalyst (such as an enzyme).

According to another aspect, this application further provides use of a diagnostic kit for diagnosing bladder and urothelial carcinoma prepared with the isolated oligonucleotides provided by this application and/or the miRNA detecting chip provided by this application.

According to another aspect, this application provides a kit for detecting a miRNA combination, including the isolated oligonucleotides provided by this application or the miRNA detecting chip provided by this application.

In some embodiments, the kit further includes one or more reagents selected from the following group: reagents for reverse transcription, reagents for RNA enrichment, reagents for cell lysis, reagents for protecting RNA from degradation, reagents for DNA amplification, and reagents for detecting DNA double-strand formation.

In some embodiments, the kit further includes a non-transient computer-readable medium, the non-transient computer-readable medium including a computer-executable instruction of calculating an expression pattern of the miRNA combination based on the expression levels of the miRNAs.

In some embodiments, the computer-executable instruction includes a classification algorithm.

In some embodiments, the classification algorithm has been subjected to training with a positive training data set and a negative training data set, the positive training data set includes the expression level of each miRNA in the miRNA combination from a plurality of urine samples of subjects with bladder and urothelial carcinoma, and the negative training data set includes the expression level of each miRNA in the miRNA combination from a plurality of urine samples of subjects without bladder and urothelial carcinoma.

According to another aspect, this application further provides a method of screening a drug candidate for treating bladder and urothelial carcinoma based on the miRNA combination provided by this application, including:

a) measuring an expression level of each miRNA in a miRNA combination from bladder and urothelial carcinoma cells in an experimental group to provide the expression level of the experimental group, and calculating the expression pattern of the miRNA combination of the experimental group based on the expression level of the experimental group to provide the expression pattern of the experimental group, where the miRNA combination includes at least 3 miRNAs selected from a group consisting of: hsa-miR-99a, hsa-miR-141, hsa-miR-151-5p, hsa-miR-96, hsa-miR-125b, hsa-miR-183, hsa-miR-1260, hsa-miR-133a, hsa-miR-429, hsa-miR-143#, hsa-miR-29c#, hsa-miR-29c, hsa-miR-152, hsa-miR-100, hsa-miR-27b, and hsa-miR-497; and the bladder and urothelial carcinoma cells in the experimental group are treated with the drug candidate;

b) measuring the expression level of each miRNA in the miRNA combination from bladder and urothelial carcinoma cells in a control group to provide the expression levels of the control group, and calculating the expression pattern of the miRNA combination of the control group based on the expression levels of the control group to provide the expression pattern of the control group, where the bladder and urothelial carcinoma cells in the control group are not treated with the drug candidate; and

c) comparing the expression patterns between the experimental group and the control group to determine whether there is a significant difference.

In some embodiments, the expression pattern is a score between 0 and 1.

In some embodiments, if a score of the experimental group is significantly lower than a score of the control group, the test substance has a potential therapeutic effect on bladder and urothelial carcinoma.

In some embodiments, the bladder and urothelial carcinoma cells in the control group are from the bladder and urothelial carcinoma cells that are not treated with the drug candidate in the experimental group.

It should be understood that, within the scope of the present disclosure, the foregoing technical features of the present disclosure and those described in detail in the following (such as examples) may be combined with each other, to form new or preferred technical solutions. For the sake of brevity, details are not described herein again.

### BRIEF DESCRIPTION OF THE DRAWINGS

FIG. 1A shows prediction results of modeling samples for a cancer group and non-cancer groups by using an SVM model of a miRNA combination 11b in 20 exemplary miRNA combinations.

FIG. 1B is an ROC curve of modeling samples of a miRNA combination 11b in 20 exemplary miRNA combinations.

FIG. 2 is a summary table showing information about detection samples.

FIG. 3A to FIG. 3D show information about 125 miRNAs, 2 endogenous controls, and 1 blank control.

FIG. 4A is a table showing information about detected modeling samples.

FIG. 4B is a table showing information about blind detection samples.

FIG. 5A is a table showing 20 exemplary miRNA combinations, where a column shows names of miRNA markers, a row shows the 20 miRNA combinations, and the meaning of each symbol “V” is that a miRNA combination corresponding to a column with “V”s includes miRNA markers corresponding to rows with the “V”s. FIG. 5B shows the sensitivity, specificity, accuracy, and relative accuracy of 20 exemplary miRNA combinations in a modeling model.

FIG. 6 shows the sensitivity, specificity, accuracy, and relative accuracy of 20 exemplary miRNA combinations in a blind detection model.

FIG. 7 shows sequences and SEQ ID NOs corresponding to 16 miRNAs in 20 exemplary miRNA combinations.

FIG. 8 shows a comparison between results of miRNA combinations used in analysis 1 to analysis 8 for modeling analysis and prediction of double-blind samples and results of an exemplary miRNA combination 11b provided by this application for modeling analysis and prediction of double-blind samples.

FIG. 9A and FIG. 9B are tables summarizing 20 miRNA combinations with good accuracy of prediction of blind samples, where a column shows names of miRNA markers, a row shows the 20 miRNA combinations, and the meaning of each symbol “V” is that a miRNA combination corresponding to a column with “V”s includes miRNA markers corresponding to rows with the “V”s.

FIG. 10 shows the difference in expression levels of 8 miRNAs in cancer samples and normal samples, and the frequency of appearance of the 8 miRNAs in 20 groups of bladder and urothelial carcinoma-specific fingerprints.

### DETAILED DESCRIPTION

It was found that some miRNA combinations are highly correlated with bladder and urothelial carcinoma. The detection of the levels of these miRNA combinations in biological samples (such as urine samples) can accurately reflect whether a subject suffers from bladder and urothelial carcinoma or is at a high risk of bladder and urothelial carcinoma, so as to provide a fingerprint of bladder and urothelial carcinoma. Based at least in part on the foregoing findings, this application provides a method for diagnosing whether a subject suffers from bladder and urothelial carcinoma or is at a high risk of bladder and urothelial carcinoma, and reagents and a kit for this diagnosis. In addition, this application further provides a method of screening a drug for treating bladder and urothelial carcinoma.

miRNA Combination

According to an aspect, this application provides a miRNA combination highly correlated with bladder and urothelial carcinoma.

As used herein, “miRNA” is a short and naturally occurring single-stranded non-coding RNA molecule with a length of about 16-26 nucleotides (nt) (for example, about 16-29 nt, 19-22 nt, 20-25 nt, or 21-23 nt), which usually functions in regulation of gene expression in the body. In this application, miRNA, small RNA, microRNA, and miR are used interchangeably and have the same meaning.

In eukaryotic cells, a miRNA gene is transcribed by DNA transcriptase II into a “primary product” (pri-miRNA), the pri-miRNA is quickly processed by a ribonuclease III (Drosha) into a miRNA “precursor” (pre-miRNA), the pre-miRNA is transported from the nucleus to the cytoplasm, and then recognized and cleaved by another ribonuclease III (Dicer) into a mature miRNA. The mature miRNA molecule is partially complementary to one or more mRNAs and regulates expression for protein. Known miRNA sequences can be obtained from public databases, for example, the miRBase database (www.mirbase.org), providing miRNA sequence information, functional annotations, predicted gene targets, and other information.

Generally, miRNA is named with the prefix of “miR” plus a number. The miRNA naming conventions may be found in the public content on www.mirbase.org, or in the article Ambros et al., RNA 9:277-279 (2003). In an example, to denote the species from which the miRNA is derived, an abbreviation referring to the species may be added in front of the name, for example, hsa refers to a miRNA derived from humans. In another example, a mature miRNA is usually named with the prefix “miR”, and its precursor or coding gene is usually named with the prefix “mir”, for example, mir-141 is the precursor of miR-141. A plurality of precursors or coding genes that form the same mature miRNA are usually distinguished by adding a numeric suffix after the name of a corresponding precursor, for example, mir-141-1 and mir-141-2 denote different precursors to miR-141. For different mature miRNAs formed by the same precursor or coding gene, in versions prior to miRBase-20, a less common mature miRNA is usually named by adding # after its name, for example, miR-141# is less common while miR-141 is more common. In versions after miRBase Version 21 (miRBase-21), a less common mature miRNA is usually named by adding * after its name, for example, miR-141* is less common. In this article, miRNA is named based on miRBase-21, that is, a less common miRNA is named with #, for example, miR-141#. With no common or uncommon distinction, a mature miRNA derived from the 5′ end of a pre-miRNA is denoted with a -5p suffix (for example, miR-151-5p), and a mature miRNA derived from the 3′ end of a pre-miRNA is denoted with a -3p suffix (for example, miR-151-3p). Two mature miRNAs with little difference in sequence may be distinguished by adding a letter suffix after a name, for example, miR-99a and miR-99b are two miRNAs with similar sequences in the same miRNA family.

The names of miRNAs provided in this application are based on the names in the miRBase database. However, it should be understood that the name of each miRNA in this application does not only denote the corresponding miRNA in the miRBase database, but also includes its pre-miRNA, pri-miRNA, splice variants, functionally equivalent mutants, homologues in different species, or derivatives.

During miRNA maturation, the splice of pre-miRNA by Dicer is not accurate in sites. Mature miRNAs with not exactly the same sequences will be produced due to different splice sites, which are also referred to as “splice variants” of the miRNA. Compared with the standard sequence of a miRNA in miRBase, its splice variant may increase or decrease several (for example, 1 to 3) nucleotides at the 3′ end. Splice variants of a miRNA may be considered to have the same function as the miRNA with the standard sequence. The miRNA recognizes a target gene through its seed region (usually 2 to 8 nucleotides at the 5′ end), so that minor differences in nucleotides in other regions do not affect the function of the miRNA. In some embodiments, a splice variant of a miRNA may include: 1) a sequence with an increase or decrease of 1 to 3 nucleotides at the 3′ end of the miRNA; or 2) a sequence with a change (such as mutation, insertion, or deletion) of 1, 2, or 3 nucleotides in regions except the seed region of 2 to 8 nucleotides at the 5′ end of the miRNA.

As used in this application, the “miRNA combination” refers to a collection of more than one (e.g., at least 3, at least 4, at least 5, at least 6, at least 7, at least 8, at least 9, at least 10, at least 11, at least 12, at least 13, at least 14, at least 15, or at least 16) miRNA. The miRNA combination provided by this application can be used to specifically and sensitively distinguish samples from subjects with bladder and urothelial carcinoma and samples from subjects without bladder and urothelial carcinoma. Therefore, the miRNA combination provided by this application may also be referred to as a miRNA fingerprint.

In some embodiments, the miRNA combination provided by this application includes at least 3 miRNAs selected from a group consisting of: hsa-miR-99a, hsa-miR-141, hsa-miR-151-5p, hsa-miR-96, hsa-miR-125b, hsa-miR-183, hsa-miR-1260, hsa-miR-133a, hsa-miR-429, hsa-miR-143#, hsa-miR-29c#, hsa-miR-29c, hsa-miR-152, hsa-miR-100, hsa-miR-27b, and hsa-miR-497. In some embodiments, the miRNA combination provided by this application may also include miRNAs of other species corresponding to the foregoing human miRNAs.

The accession numbers and standard sequences of the foregoing miRNAs in miRBase are shown in the following table A. However, as described above, the foregoing miRNA names do not only denote the miRNAs with the standard sequences shown in the table A, but also include their pre-miRNA, pri-miRNA, splice variants, functionally equivalent mutants, homologues in different species, and derivatives.

TABLE A
Standard sequence of miRNA in miRNA combination

| SEQ ID | | Mature miRNA | |
|---|---|---|---|
| NO.: | miRNA name | accession number | Mature miRNA sequence (5′-3′) |
| 1 | hsa-miR-99a | MIMAT0000097 | AACCCGUAGAUCCGAUCUUGUG |
| 2 | hsa-miR-141 | MIMAT0000432 | UAACACUGUCUGGUAAAGAUGG |
| 3 | hsa-miR-151-5p | MIMAT0004697 | UCGAGGAGCUCACAGUCUAGU |
| 4 | hsa-miR-96 | MIMAT0000095 | UUUGGCACUAGCACAUUUUUGCU |
| 5 | hsa-miR-125b | MIMAT0000423 | UCCCUGAGACCCUAACUUGUGA |
| 6 | hsa-miR-183 | MIMAT0000261 | UAUGGCACUGGUAGAAUUCACU |
| 7 | hsa-miR-1260 | MIMAT0005911 | AUCCCACCUCUGCCACCA |
| 8 | hsa-miR-133a | MIMAT0000427 | UUUGGUCCCCUUCAACCAGCUG |
| 9 | hsa-miR-429 | MIMAT0001536 | UAAUACUGUCUGGUAAAACCGU |
| 10 | hsa-miR-143# | MIMAT0004599 | GGUGCAGUGCUGCAUCUCUGGU |
| 11 | hsa-miR-29c# | MIMAT0004673 | UGACCGAUUUCUCCUGGUGUUC |
| 12 | hsa-miR-29c | MIMAT0000681 | UAGCACCAUUUGAAAUCGGUUA |
| 13 | hsa-miR-152 | MIMAT0000438 | UCAGUGCAUGACAGAACUUGG |
| 14 | hsa-miR-100 | MIMAT0000098 | AACCCGUAGAUCCGAACUUGUG |
| 15 | hsa-miR-27b | MIMAT0000419 | UUCACAGUGGCUAAGUUCUGC |
| 16 | hsa-miR-497 | MIMAT0002820 | CAGCAGCACACUGUGGUUUGU |

In some embodiments, the miRNA combination includes at least 3, at least 4, at least 5, at least 6, at least 7, at least 8, at least 9, at least 10, at least 11, at least 12, at least 13, at least 14, at least 15, or at least 16 miRNAs selected from a group consisting of: hsa-miR-99a, hsa-miR-141, hsa-miR-151-5p, hsa-miR-96, hsa-miR-125b, hsa-miR-183, hsa-miR-1260, hsa-miR-133a, hsa-miR-429, hsa-miR-143#, hsa-miR-29c#, hsa-miR-29c, hsa-miR-152, hsa-miR-100, hsa-miR-27b, and hsa-miR-497.

In some embodiments, the miRNA combination includes at least 3, at least 4, at least 5, at least 6, at least 7, at least 8, at least 9, at least 10, at least 11, at least 12, at least 13, at least 14, or at least 15 miRNAs selected from a group consisting of: hsa-miR-99a, hsa-miR-141, hsa-miR-151-5p, hsa-miR-96, hsa-miR-125b, hsa-miR-183, hsa-miR-1260, hsa-miR-133a, hsa-miR-429, hsa-miR-143#, hsa-miR-29c#, hsa-miR-29c, hsa-miR-152, hsa-miR-100, and hsa-miR-27b.

In some embodiments, the miRNA combination includes at least 3, at least 4, at least 5, at least 6, at least 7, at least 8, at least 9, at least 10, at least 11, at least 12, at least 13, or at least 14 miRNAs selected from a group consisting of: hsa-miR-99a, hsa-miR-141, hsa-miR-151-5p, hsa-miR-96, hsa-miR-125b, hsa-miR-183, hsa-miR-1260, hsa-miR-133a, hsa-miR-429, hsa-miR-143#, hsa-miR-29c#, hsa-miR-29c, hsa-miR-152, and hsa-miR-100.

In some embodiments, the miRNA combination includes at least 3, at least 4, at least 5, at least 6, at least 7, at least 8, at least 9, at least 10, at least 11, at least 12, or at least 13 miRNAs selected from a group consisting of: hsa-miR-99a (or hsa-miR-100), hsa-miR-141, hsa-miR-151-5p, hsa-miR-96, hsa-miR-125b, hsa-miR-183, hsa-miR-1260, hsa-miR-133a, hsa-miR-429, hsa-miR-143#, hsa-miR-29c#, hsa-miR-29c, and hsa-miR-152.

In some embodiments, the miRNA combination includes at least 3, at least 4, at least 5, at least 6, at least 7, at least 8, at least 9, at least 10, at least 11, or at least 12 miRNAs selected from a group consisting of: hsa-miR-99a (or hsa-miR-100), hsa-miR-141, hsa-miR-151-5p, hsa-miR-96, hsa-miR-125b, hsa-miR-183, hsa-miR-1260, hsa-miR-133a, hsa-miR-429, hsa-miR-143#, hsa-miR-29c#, and hsa-miR-29c.

In some embodiments, the miRNA combination includes at least 3, at least 4, at least 5, at least 6, at least 7, at least 8, at least 9, at least 10, or at least 11 miRNAs selected from a group consisting of: hsa-miR-99a (or hsa-miR-100), hsa-miR-141, hsa-miR-151-5p, hsa-miR-96, hsa-miR-125b, hsa-miR-183, hsa-miR-1260, hsa-miR-133a, hsa-miR-429, hsa-miR-143#, and hsa-miR-29c#.

In some embodiments, the miRNA combination includes at least 3, at least 4, at least 5, at least 6, at least 7, at least 8, at least 9, or at least 10 miRNAs selected from a group consisting of: hsa-miR-99a (or hsa-miR-100), hsa-miR-141, hsa-miR-151-5p, hsa-miR-96, hsa-miR-125b, hsa-miR-183, hsa-miR-1260, hsa-miR-133a, hsa-miR-429, and hsa-miR-143#.

In some embodiments, the miRNA combination includes at least 3, at least 4, at least 5, at least 6, at least 7, at least 8, at least 9, or at least 10 miRNAs selected from a group consisting of: hsa-miR-100, hsa-miR-125b, hsa-miR-133a, hsa-miR-141, hsa-miR-143#, hsa-miR-151-5p, hsa-miR-29c, hsa-miR-29c#, hsa-miR-99a, and hsa-miR-96.

In some embodiments, the miRNA combination includes at least 3, at least 4, at least 5, at least 6, at least 7, at least 8, or at least 9 miRNAs selected from a group consisting of: hsa-miR-99a (or hsa-miR-100), hsa-miR-141, hsa-miR-151-5p, hsa-miR-96, hsa-miR-125b, hsa-miR-183, hsa-miR-1260, hsa-miR-133a, and hsa-miR-429.

In some embodiments, the miRNA combination includes at least 3, at least 4, at least 5, at least 6, at least 7, at least 8, or at least 9 miRNAs selected from a group consisting of: hsa-miR-100, hsa-miR-125b, hsa-miR-133a, hsa-miR-141, hsa-miR-143#, hsa-miR-151-5p, hsa-miR-29c, hsa-miR-99a, and hsa-miR-29c#.

In some embodiments, the miRNA combination includes at least 3, at least 4, at least 5, at least 6, at least 7, at least 8, or at least 9 miRNAs selected from a group consisting of: hsa-miR-100, hsa-miR-125b, hsa-miR-133a, hsa-miR-141, hsa-miR-143#, hsa-miR-151-5p, hsa-miR-29c, hsa-miR-99a, and hsa-miR-96.

In some embodiments, the miRNA combination includes at least 3, at least 4, at least 5, at least 6, at least 7, or at least 8 miRNAs selected from a group consisting of: hsa-miR-99a (or hsa-miR-100), hsa-miR-141, hsa-miR-151-5p, hsa-miR-96, hsa-miR-125b, hsa-miR-183, hsa-miR-1260, and hsa-miR-133a.

In some embodiments, the miRNA combination includes at least 3, at least 4, at least 5, at least 6, at least 7, or at least 8 miRNAs selected from a group consisting of: hsa-miR-99a, hsa-miR-141, hsa-miR-151-5p, hsa-miR-143#, hsa-miR-125b, hsa-miR-29c, hsa-miR-100, and hsa-miR-133a.

In some embodiments, the miRNA combination includes at least 3, at least 4, at least 5, at least 6, or at least 7 miRNAs selected from a group consisting of: hsa-miR-99a (or hsa-miR-100), hsa-miR-141, hsa-miR-151-5p, hsa-miR-96, hsa-miR-125b, hsa-miR-183, and hsa-miR-1260.

In some embodiments, the miRNA combination includes at least 3, at least 4, at least 5, at least 6, or at least 7 miRNAs selected from a group consisting of: hsa-miR-141, hsa-miR-151-5p, hsa-miR-143#, hsa-miR-125b, hsa-miR-29c, hsa-miR-100 (or hsa-miR-99a), and hsa-miR-133a.

In some embodiments, the miRNA combination includes at least 3, at least 4, at least 5, or at least 6 miRNAs selected from a group consisting of: hsa-miR-99a (or hsa-miR-100), hsa-miR-141, hsa-miR-151-5p, hsa-miR-96, hsa-miR-125b, and hsa-miR-183.

In some embodiments, the miRNA combination includes at least 3, at least 4, or at least 5 miRNAs selected from a group consisting of: hsa-miR-99a (or hsa-miR-100), hsa-miR-141, hsa-miR-151-5p, hsa-miR-96, and hsa-miR-125b.

In some embodiments, the miRNA combination includes at least 3, or at least 4 miRNAs selected from a group consisting of: hsa-miR-99a (or hsa-miR-100), hsa-miR-141, hsa-miR-151-5p, and hsa-miR-96.

In some embodiments, the miRNA combination includes the following miRNA combination: hsa-miR-99a, hsa-miR-141, and hsa-miR-151-5p; or includes the following miRNA combination: hsa-miR-100, hsa-miR-141, and hsa-miR-151-5p. In some embodiments, the miRNA combination includes hsa-miR-99a. In some embodiments, the miRNA combination further includes hsa-miR-141 and/or hsa-miR-151-5p. In some embodiments, the miRNA combination further includes hsa-miR-96, and/or further includes hsa-miR-125b, and/or further includes hsa-miR-183, and/or further includes hsa-miR-1260, and/or further includes hsa-miR-133a, and/or further includes hsa-miR-429, and/or further includes hsa-miR-143#, and/or further includes hsa-miR-29c#, and/or further includes hsa-miR-29c, and/or further includes hsa-miR-152, and/or further includes hsa-miR-100, and/or further includes hsa-miR-29b, and/or further includes hsa-miR-497.

In some embodiments, the miRNA combination includes hsa-miR-99a, hsa-miR-141, hsa-miR-151-5p, hsa-miR-96, hsa-miR-125b, hsa-miR-183, and hsa-miR-133a. In some embodiments, the miRNA combination includes hsa-miR-99a, hsa-miR-141, hsa-miR-151-5p, hsa-miR-96, hsa-miR-125b, hsa-miR-183, hsa-miR-1260, hsa-miR-133a, and hsa-miR-429.

In some embodiments, the miRNA combination includes any one of miRNA combinations selected from combination 1, combination 2, combination 3, combination 4, combination 5, combination 6, combination 7, combination 8, combination 9, combination 10, combination 11, combination 12, combination 13, combination 14, combination 15, combination 16, combination 17, combination 18, combination 19, combination 20, combination 21, and combination 22, where:

the combination 1 includes: hsa-miR-99a, hsa-miR-141, hsa-miR-151-5p, hsa-miR-96, hsa-miR-125b, hsa-miR-183, hsa-miR-1260, hsa-miR-143#, hsa-miR-29c#, hsa-miR-152, and hsa-miR-100;

the combination 2 includes: hsa-miR-99a, hsa-miR-141, hsa-miR-151-5p, hsa-miR-96, hsa-miR-125b, hsa-miR-183, hsa-miR-1260, hsa-miR-143#, hsa-miR-29c#, hsa-miR-29c, hsa-miR-152, and hsa-miR-100;

the combination 3 includes: hsa-miR-99a, hsa-miR-141, hsa-miR-151-5p, hsa-miR-1260, hsa-miR-133a, hsa-miR-429, hsa-miR-143#, hsa-miR-29c#, hsa-miR-29c, and hsa-miR-152; or includes: hsa-miR-100, hsa-miR-141, hsa-miR-151-5p, hsa-miR-1260, hsa-miR-133a, hsa-miR-429, hsa-miR-143#, hsa-miR-29c#, hsa-miR-29c, and hsa-miR-152;

the combination 4 includes: hsa-miR-99a, hsa-miR-96, hsa-miR-125b, hsa-miR-183, hsa-miR-429, hsa-miR-143#, hsa-miR-29c#, and hsa-miR-27b; or includes: hsa-miR-100, hsa-miR-96, hsa-miR-125b, hsa-miR-183, hsa-miR-429, hsa-miR-143#, hsa-miR-29c#, and hsa-miR-27b;

the combination 5 includes: hsa-miR-99a, hsa-miR-96, hsa-miR-125b, hsa-miR-183, hsa-miR-429, hsa-miR-143#, hsa-miR-29c#, and hsa-miR-152; or includes: hsa-miR-100, hsa-miR-96, hsa-miR-125b, hsa-miR-183, hsa-miR-429, hsa-miR-143#, hsa-miR-29c#, and hsa-miR-152;

the combination 6 includes: hsa-miR-99a, hsa-miR-141, hsa-miR-151-5p, hsa-miR-96, hsa-miR-183, hsa-miR-1260, hsa-miR-133a, hsa-miR-143#, hsa-miR-29c, and hsa-miR-27b; or includes: hsa-miR-100, hsa-miR-141, hsa-miR-151-5p, hsa-miR-96, hsa-miR-183, hsa-miR-1260, hsa-miR-133a, hsa-miR-143#, hsa-miR-29c, and hsa-miR-27b;

the combination 7 includes: hsa-miR-99a, hsa-miR-141, hsa-miR-151-5p, hsa-miR-1260, hsa-miR-429, hsa-miR-143#, hsa-miR-29c#, hsa-miR-29c, and hsa-miR-152; or includes: hsa-miR-100, hsa-miR-141, hsa-miR-151-5p, hsa-miR-1260, hsa-miR-429, hsa-miR-143#, hsa-miR-29c#, hsa-miR-29c, and hsa-miR-152;

the combination 8 includes: hsa-miR-99a, hsa-miR-141, hsa-miR-151-5p, hsa-miR-96, hsa-miR-125b, hsa-miR-1260, hsa-miR-133a, hsa-miR-429, hsa-miR-143#, hsa-miR-29c#, hsa-miR-29c, and hsa-miR-152; or includes: hsa-miR-100, hsa-miR-141, hsa-miR-151-5p, hsa-miR-96, hsa-miR-125b, hsa-miR-1260, hsa-miR-133a, hsa-miR-429, hsa-miR-143#, hsa-miR-29c#, hsa-miR-29c, and hsa-miR-152;

the combination 9 includes: hsa-miR-99a, hsa-miR-141, hsa-miR-151-5p, hsa-miR-96, hsa-miR-125b, hsa-miR-183, hsa-miR-1260, hsa-miR-143#, hsa-miR-29c#, hsa-miR-29c, hsa-miR-152, and hsa-miR-100;

the combination 10 includes: hsa-miR-99a, hsa-miR-141, hsa-miR-151-5p, hsa-miR-96, hsa-miR-125b, hsa-miR-183, hsa-miR-1260, hsa-miR-133a, hsa-miR-143#, hsa-miR-29c#, hsa-miR-29c, hsa-miR-152, and hsa-miR-27b; or includes: hsa-miR-100, hsa-miR-141, hsa-miR-151-5p, hsa-miR-96, hsa-miR-125b, hsa-miR-183, hsa-miR-1260, hsa-miR-133a, hsa-miR-143#, hsa-miR-29c#, hsa-miR-29c, hsa-miR-152, and hsa-miR-27b;

the combination 11 includes: hsa-miR-99a, hsa-miR-141, hsa-miR-151-5p, hsa-miR-96, hsa-miR-125b, hsa-miR-183, hsa-miR-1260, hsa-miR-133a, and hsa-miR-429; or includes: hsa-miR-100, hsa-miR-141, hsa-miR-151-5p, hsa-miR-96, hsa-miR-125b, hsa-miR-183, hsa-miR-1260, hsa-miR-133a, and hsa-miR-429;

the combination 12 includes: hsa-miR-99a, hsa-miR-141, hsa-miR-151-5p, hsa-miR-96, hsa-miR-125b, hsa-miR-183, and hsa-miR-133a; or includes: hsa-miR-100, hsa-miR-141, hsa-miR-151-5p, hsa-miR-96, hsa-miR-125b, hsa-miR-183, and hsa-miR-133a;

the combination 13 includes: hsa-miR-99a, hsa-miR-141, hsa-miR-151-5p, hsa-miR-96, hsa-miR-125b, hsa-miR-183, hsa-miR-1260, hsa-miR-133a, hsa-miR-429, and hsa-miR-29c; or includes: hsa-miR-100, hsa-miR-141, hsa-miR-151-5p, hsa-miR-96, hsa-miR-125b, hsa-miR-183, hsa-miR-1260, hsa-miR-133a, hsa-miR-429, and hsa-miR-29c;

the combination 14 includes: hsa-miR-99a, hsa-miR-141, hsa-miR-151-5p, hsa-miR-96, hsa-miR-125b, hsa-miR-183, hsa-miR-1260, hsa-miR-133a, hsa-miR-429, and hsa-miR-497; or includes: hsa-miR-100, hsa-miR-141, hsa-miR-151-5p, hsa-miR-96, hsa-miR-125b, hsa-miR-183, hsa-miR-1260, hsa-miR-133a, hsa-miR-429, and hsa-miR-497;

the combination 15 includes: hsa-miR-99a, hsa-miR-141, hsa-miR-151-5p, hsa-miR-96, hsa-miR-125b, hsa-miR-183, hsa-miR-1260, hsa-miR-133a, hsa-miR-429, hsa-miR-143#, and hsa-miR-29c; or includes: hsa-miR-100, hsa-miR-141, hsa-miR-151-5p, hsa-miR-96, hsa-miR-125b, hsa-miR-183, hsa-miR-1260, hsa-miR-133a, hsa-miR-429, hsa-miR-143#, and hsa-miR-29c;

the combination 16 includes: hsa-miR-99a, hsa-miR-141, hsa-miR-151-5p, hsa-miR-96, hsa-miR-125b, hsa-miR-133a, hsa-miR-143#, and hsa-miR-29c#; or includes: hsa-miR-100, hsa-miR-141, hsa-miR-151-5p, hsa-miR-96, hsa-miR-125b, hsa-miR-133a, hsa-miR-143#, and hsa-miR-29c#;

the combination 17 includes: hsa-miR-99a, hsa-miR-141, hsa-miR-151-5p, hsa-miR-96, hsa-miR-125b, hsa-miR-183, hsa-miR-133a, hsa-miR-429, hsa-miR-143#, and hsa-miR-29c#; or includes: hsa-miR-100, hsa-miR-141, hsa-miR-151-5p, hsa-miR-96, hsa-miR-125b, hsa-miR-183, hsa-miR-133a, hsa-miR-429, hsa-miR-143#, and hsa-miR-29c#;

the combination 18 includes: hsa-miR-100, hsa-miR-125b, hsa-miR-133a, hsa-miR-141, hsa-miR-143#, hsa-miR-151-5p, hsa-miR-29c, hsa-miR-29c#, hsa-miR-99a, and hsa-miR-96;

the combination 19 includes: hsa-miR-100, hsa-miR-125b, hsa-miR-133a, hsa-miR-141, hsa-miR-143#, hsa-miR-151-5p, hsa-miR-29c, and hsa-miR-99a;

the combination 20 includes: hsa-miR-100, hsa-miR-125b, hsa-miR-133a, hsa-miR-141, hsa-miR-143#, hsa-miR-151-5p, hsa-miR-29c, hsa-miR-99a, and hsa-miR-29c#;

the combination 21 includes: hsa-miR-100, hsa-miR-125b, hsa-miR-133a, hsa-miR-141, hsa-miR-143#, hsa-miR-151-5p, hsa-miR-29c, hsa-miR-99a, and hsa-miR-96; and

the combination 22 includes: hsa-miR-141, hsa-miR-151-5p, hsa-miR-143#, hsa-miR-125b, hsa-miR-29c, hsa-miR-100, and hsa-miR-133a; or includes: hsa-miR-141, hsa-miR-151-5p, hsa-miR-143#, hsa-miR-125b, hsa-miR-29c, hsa-miR-99a, and hsa-miR-133a.

According to an aspect, this application provides a mixture containing the foregoing miRNA combination or the oligonucleotides coding the foregoing miRNA combination. In some embodiments, the mixture contains more or all of miRNAs in the foregoing miRNA combination provided by this application. In some embodiments, the mixture contains cDNAs of more or all of miRNAs in the foregoing miRNA combination provided by this application. Such mixture can be used as a standard or reference in testing.

Reagent for Detecting miRNA Combination

According to an aspect, this application provides one or more isolated oligonucleotides including a hybridization region, where the hybridization region can hybridize with a miRNA in a miRNA combination provided by this application or a complement sequence thereof. In some embodiments, this application provides one or more sets of isolated oligonucleotides, where each of the oligonucleotides includes a hybridization region, and the hybridization region can hybridize with a corresponding miRNA in a miRNA combination provided by this application or a complement sequence thereof.

In this application, “hybridization,” “hybridize,” or “hybridizing” refer to specific binding or double-strand formation with a target sequence through at least partial base pairing under proper reaction conditions substantially other than that with other nucleic acid sequences from a mixture (for example, from a cell lysate or a DNA preparation). Those skilled in the art may select proper hybridization reaction conditions according to the length and sequence of a target sequence. There are a large number of public teachings on nucleic acid hybridization in the art, for example, refer to Tijssen Laboratory Techniques in Biochemistry and Molecular Biology—Hybridization with Nucleic Acid Probes part I, Ch. 2, “Overview of principles of hybridization and the strategy of nucleic acid probe assays,” (1993) Elsevier, N.Y.

In some embodiments, the hybridization region of each of the oligonucleotides is complementary to the nucleotide sequence of the corresponding miRNA or a complement sequence thereof. The term “complementary” or “complementarity” refer to the capability of a nucleic acid sequence to form hydrogen bonds with another nucleic acid sequence through conventional Watson-Crick base pairing or another non-conventional pairing. Complementarity in percentage refers to a percentage of nucleotides in a first nucleic acid molecule that can form hydrogen bonds (for example, Watson-Crick bonds) with a second nucleic acid sequence. For example, if 5, 6, 7, 8, 9, or 10 out of 10 nucleotides form hydrogen bonds with the second nucleic acid sequence, the complementarity in percentage is 50%, 60%, 70%, 80%, 90%, or 100%. In some embodiments, in a sequence length of at least 7 nucleotides, more typically a sequence length of 10-30 nucleotides, and usually a sequence length of at least 14-25 nucleotides, the hybridization region is at least 50%, at least 55%, at least 60%, at least 65%, at least 70%, at least 80%, at least 85%, at least 90%, at least 95%, at least 99%, or 100% complementary to the nucleotide sequence of the corresponding miRNA or a complement sequence thereof.

In some embodiments, the hybridization region of each of the oligonucleotides is substantially complementary to the nucleotide sequence of the corresponding miRNA or a complement sequence thereof. In some embodiments, the substantial complementation refers to including no more than 1, 2, or 3 base mispairings.

In some embodiments, this application provides a set of isolated oligonucleotides including a hybridization region, where the hybridization region in each of the oligonucleotides can hybridize with a corresponding miRNA in a miRNA combination or a complement sequence thereof. In some embodiments, the miRNA combination includes: at least 3, at least 4, at least 5, at least 6, at least 7, at least 8, at least 9, at least 10, at least 11, at least 12, at least 13, at least 14, at least 15, or at least 16 miRNAs selected from SEQ ID NOs: 1-16, miRNAs selected from SEQ ID NOs: 1-15, miRNAs selected from SEQ ID NOs: 1-14, miRNAs selected from SEQ ID NOs: 1-13, miRNAs selected from SEQ ID NOs: 1-12, miRNAs selected from SEQ ID NOs: 1-11, miRNAs selected from SEQ ID NOs: 1-10, miRNAs selected from SEQ ID NOs: 1-9, miRNAs selected from SEQ ID NOs: 1-8, miRNAs selected from SEQ ID NOs: 1-7, miRNAs selected from SEQ ID NOs: 1-6, miRNAs selected from SEQ ID NOs: 1-5, miRNAs selected from SEQ ID NOs: 1-4, miRNAs selected from SEQ ID NOs: 1-5, 8, 10-12, and 14, miRNAs selected from SEQ ID NOs: 1-3, 5, 8, 10-12, and 14, miRNAs selected from SEQ ID NOs: 1-5, 8, 10, 12, and 14, miRNAs selected from SEQ ID NOs: 2, 3, 5, 8, 10, 12, and 14, or miRNAs selected from SEQ ID NOs: 1-3, 5, 8, 10, 12, and 14. In some embodiments, the miRNA combination further includes at least one miRNA selected from the following or any combination thereof: SEQ ID NOs: 1-3. In some embodiments, the miRNA combination further includes SEQ ID NO: 1. In some embodiments, the isolated oligonucleotides provided by this application can be used to detect one or more miRNAs in the miRNA combination provided by this application. In some embodiments, the isolated oligonucleotide includes a primer or a probe.

In this application, the “primer” refers to an oligonucleotide sequence that can specifically hybridize with a target nucleic acid and allow the target nucleic acid to be amplified. A primer usually includes 7-40 nucleotides, 10-38 nucleotides, 15-30 nucleotides, 15-25 nucleotides, or 17-20 nucleotides. For example, the primer may be an oligonucleotide or a polynucleotide with a length of 7, 8, 9, 10, 11, 12, 13, 14, 15, 16, 17, 18, 19, 20, 21, 22, 23, 24, 25, 26, 27, 28, 29, or 30. The primer may include DNA, RNA, nucleic acid analogues, or any combination thereof. For example, the primer may be chemically synthesized. It should be understood that it is generally expected that some bases (for example, a base at the 3′ end of a primer) are completely complementary to corresponding bases of a target nucleic acid sequence to facilitate the elongation of the primer in the amplification reaction. In some embodiments, a primer may also protect the part that does not specifically hybridize with a target nucleic acid, such as a nucleic acid sequence for immobilization or labeling.

In this application, the “probe” refers to an oligonucleotide that can bind to a target nucleic acid with at least partial complementary sequences using one or more types of chemical bonds (usually through base pairing, for example, through the formation of hydrogen bonds) to form a double-strand structure. The probe may be a DNA probe, an RNA probe, or a protein nucleic acid (PNA) probe. The size of the probe may change. Generally, the length of a probe may be at least 7-15 nucleotides, or may be at least 20, 30, or 40 nucleotides, or may be at least 50, 60, 70, 80, or 90 nucleotides, or may be at least 100, 150, 200, or more nucleotides. A probe may have any length within any range bounded by any of the foregoing values (for example, 15-20 nucleotides).

Sequences of a primer and a probe may be designed according to a sequence of a test miRNA or a complement sequence thereof. In some embodiments, a hybridization region of a primer used for amplification is usually a sequence completely complementary to a miRNA.

In some embodiments, the isolated oligonucleotides further include a reference oligonucleotide that can hybridize with an endogenous reference, such as a reference oligonucleotide primer or a reference oligonucleotide probe.

In some embodiments, the oligonucleotide primer or the oligonucleotide probe further includes a detectable marker. In this application, the “detectable marker” refers to a marker that can be detected or can be allowed to be detected. The detectable marker can be used to label a probe to allow convenient probe detection, especially with the probe hybridized with its target complement sequence. As described in chapter 10 and chapter 11 of Molecular Cloning: A Laboratory Manual, J. Sambrook et al., eds., 2nd edition, Cold Spring Harbor Laboratory Press, 1989, a method for preparing a labeled DNA and RNA probe and conditions for hybridization with a target nucleotide sequence are incorporated herein by reference.

In some embodiments, the detectable marker includes, but is not limited to, a chromophore, an isotopic label, a heavy metal, a fluorophore, a chemiluminescent group, visible or fluorescent particles, nucleic acids, a binding ligand, or a catalyst (such as an enzyme).

The chromophore includes the digoxin (DIG) molecule.

The isotopic label includes, but is not limited to, ³H, ³²P, ³³P, ¹⁴C, ³⁵S, ¹²³I, ¹²⁴I, ¹²⁵I, ¹³¹I, ³⁵S, ³H, ¹¹¹In, ¹¹²In, ¹⁴C, ⁶⁴Cu, ⁶⁷Cu, ⁸⁶Y, ⁸⁸Y, ⁹⁰Y, ¹⁷⁷Lu, ²¹¹At, ¹⁸⁶Re, ¹⁸⁸Re, ¹⁵³Sm, ²¹²Bi, and ³²P.

The fluorophore includes, but is not limited to, acridine, 7-amino-4-methylcoumarin-3-aceticacid (AMCA), BODIPY, Cascade Blue, Cy2, Cy3, Cy5, Cy7, Edans, Eosin, Erythrosin, Fluorescein, 6-carboxyfluorescein, 6-TET, JOC, HEX, Oregon Green, Rhodamine, Rhodol Green, Tamra. Rox, and Texas Red (Molecular Probes, Eugene, Oreg.).

The heavy metal includes nano gold.

The ligand includes, but is not limited to, biotin, avidin, antibody, or antigen.

The enzyme includes, but is not limited to, alkaline phosphatase, acid phosphatase, horseradish peroxidase, β-galactosidase, and ribonuclease.

It should be understood that the detectable marker itself does not necessarily generate a detectable signal. For example, in some embodiments, the detectable marker may react with a detectable ligand or react with one or more other compounds to generate a detectable signal. For example, the detectable marker may be a ligand that can specifically bind to another labeled ligand (such as a labeled secondary antibody). In another example, an enzyme may be used as a detectable marker. It can catalyze substrates that can provide color, fluorescence, or chemiluminescence due to its catalytic activity, thereby generating a detectable signal.

In some embodiments, a primer or a probe of the detectable marker may further include a quencher. The quencher refers to a group that quenches the fluorescence emitted by a fluorophore to which the quencher is close sufficiently due to, for example, fluorescence resonance energy transfer (FRET).

The quencher includes, but is not limited to, Tamra, Dabcyl, Black Hole Quencher (BHQ, Biosearch Technologies), DDQ (Eurogentec), Iowa Black FQ (Integrated DNA Technologies), QSY-7 (Molecular Probes), and Eclipse quencher (Epoch Biosciences).

In some embodiments, the primer or the probe includes a fluorophore and further includes a quencher.

In some other embodiments, the primer or the probe may not include a detectable marker, that is, the primer or the probe is an unlabeled primer or probe. The unlabeled probe may specifically bind to a labeled ligand or a labeled test substance directly or indirectly to allow the test substance to be detected. In an example, a test target nucleic acid labeled by a detectable marker can be detected when hybridizing with an unlabeled probe. In another example, the dTTP analogue 5-(N-(n-biotin-ε-aminohexyl)-3-aminoallyl) deoxyuridine triphosphate may be incorporated into a probe molecule to obtain a biotinylated probe that can bind to a biotin-binding protein (such as avidin, streptavidin, and antibody (for example, anti-biotin antibody)), and the biotin-binding protein can be further labeled with a fluorescent dye or enzyme that produces a color reaction.

In some embodiments, the isolated oligonucleotides are immobilized on a solid support. The solid support may be modified to contain discrete independent sites suitable for attachment or binding of the isolated oligonucleotides, and is suitable for at least one detection method. Representatively, the solid support includes glass and modified or functionalized glass, plastics (including acrylic resin, polystyrene, and copolymers of styrene and other materials, polypropylene, polyethylene, polybutene, polyurethane, TeflonJ, etc.), polysaccharides, nylon or nitrocellulose, resin, silica or silica-based materials, silicon and modified silicon, carbon, metals, inorganic glass and plastics. The support allows optical detection without emission of significant fluorescence.

Diagnosis Method

According to an aspect, this application provides a method for diagnosing whether a test subject suffers from bladder and urothelial carcinoma or is at a high risk of bladder and urothelial carcinoma, including:

a) obtaining a test urine sample from the test subject;

b) measuring an expression level of each miRNA in a miRNA combination provided by this application in the test urine sample; and

c) assessing whether the test subject suffers from bladder and urothelial carcinoma or is at a high risk of bladder and urothelial carcinoma, based on the expression levels of the miRNAs.

According to an aspect, this application provides use of a set of oligonucleotides including a hybridization region in preparing a kit. The kit is used for diagnosing whether a test subject suffers from bladder and urothelial carcinoma or is at a high risk of bladder and urothelial carcinoma. The hybridization region in each of the oligonucleotides can hybridize with a corresponding miRNA in a miRNA combination provided by this application or a complement sequence thereof. In some embodiments, the kit is used in the diagnosis method provided by this application.

1. Obtaining Samples

In the method of this application, a test urine sample of a test subject is used. The urine sample includes, but is not limited to: whole urine, urine sediment, urine supernatant, the cells contained in urine, or RNAs isolated from one or more of the foregoing samples.

In some embodiments, the method further includes enriching RNAs from the test urine sample. For example, the urine sample may be concentrated to enrich RNAs therein, or may be centrifuged.

In some embodiments, the enriching includes extracting RNAs from a precipitate obtained by centrifugating the test urine sample. The methods for the extraction of RNAs is well known to a person skilled in the art, for example, the TRIzol method. TRIzol is a new total RNA extraction reagent containing phenol and guanidine thiocyanate, and can quickly break cells and inhibit the nucleases released from the cells to keep RNA intact. The total RNA can be extracted from cells or tissues by the TRIzol method.

In some embodiments, the enrichment further includes isolating RNAs with a specific length from the test urine sample, for example, a small RNA segment generally ranging from 10 to 100 bases in length. Enriching small RNAs segments for the subsequent detection of the expression levels can improve the accuracy of miRNA capture and detection. A person skilled in the art can easily isolate RNAs with a specific segment length by affinity column chromatography, gel electrophoresis, magnetic bead capture, or the like. The miRNAs may also be purified from the urine sample by using a conventional kit. Representatively, a kit for extraction of miRNA includes, but is not limited to, a miRNA extraction kit from Qiagen.

In some embodiments, it further includes introducting a detectable marker into the enriched RNAs. Any detectable markers suitable for labeling nucleic acids may be used, including but not limited to the detectable markers provided by this application.

2. Measuring miRNA Expression Level

The method provided by this application further includes measuring the expression level of each miRNA in the miRNA combination provided by this application in the test urine sample.

In this application, the “expression level” of a miRNA refers to the measured amount, concentration, or relative abundance of the miRNA in a sample. The expression levels of miRNAs may be measured by a proper method, including but not limited to an amplification-based method, a hybridization-based method, and/or a sequencing-based method.

Amplification-Based Method

The amplification-based method refers to a method including an amplification reaction of nucleic acids. Nucleic acid amplification measurement involves replication of target nucleic acids (such as DNA or RNA), so as to increase the quantity of amplified nucleic acid sequences. The amplification may be exponential or linear. For example, the nucleic acid amplification method includes, but is not limited to, polymerase chain reaction (PCR, referring to U.S. Pat. Nos. 4,683,195 and 4,683,202; PCR Protocols: A Guide To Methods And Applications (Innis et al., eds, 1990)), reverse transcription polymerase chain reaction (RT-PCR), real-time quantitative PCR (qRT-PCR), quantitative PCR such as fluorescent dye quantitative PCR, TaqMan®, nested PCR, ligase chain reaction (referring to Abravaya, K., et al., Nucleic Acids Research, 23:675-682, (1995)), branched DNA signal amplification (referring to Urdea, M. S., et al., AIDS, 7 (suppl 2):S11-S14, (1993)), amplifiable RNA reporters, Q-β replication (referring to Lizardi et al., Biotechnology (1988) 6: 1197), transcription-based amplification (referring to Kwoh et al., Proc. Natl. Acad. Sci. USA (1989) 86: 1173-1177), boomerang DNA amplification, strand displacement activation, cycling probe technology, self-sustained sequence replication (referring to Guatelli et al., Proc. Natl. Acad. Sci. USA (1990) 87: 1874-1878), rolling circle replication (referring to U.S. Pat. No. 5,854,033), nucleic acid sequence based amplification (NASBA), serial analysis of gene expression (SAGE), and the like. For example, an amplification technology with high sensitivity and specificity disclosed in CN10267663A may also be used in the present disclosure. These methods are all suitable for amplifying a miRNA, a precursor thereof, a coding gene thereof, or a reverse-transcribed DNA thereof.

In some embodiments, the amplification reaction of nucleic acids includes a PCR-based method. In some embodiments, an expression level of the miRNA is measured by the PCR-based method. PCR is a method for amplifying nucleic acids (such as DNA or RNA) in vitro by synthesizing a new nucleic acid strand complementary to a target nucleic acid as a template through elongating one, two, or more primers that can hybridize with the target nucleic acid under the catalysis of an enzyme.

In some embodiments, an expression level of each miRNA in this application may be measured by reverse transcription PCR. In such embodiments, before the expression level of each miRNA is measured, the method further includes reverse transcription of each miRNA into cDNA. The reverse transcription may be carried out by a method known in the art, for example, the miRNA is polyA-tailed (by the polyA polymerase), and then the reverse transcription is carried out with polyT as a primer; in another example, the miRNA may be ligated with an adapter sequence (by T4 RNA ligase), and then the reverse transcription is carried out with a primer complementary to the adapter sequence. Various reverse transcriptases may be used, including but not limited to MMLV RT, RNase H mutants of MMLV RT such as Superscript and Superscript II (Life Technologies, GIBCO BRL, Gaithersburg, Md.), AMV RT, and thermostable reverse transcriptase from thermophile. For example, a method for reverse transcription of RNA into cDNA is adapted from a scheme of the Superscript II pre-amplification system (Life Technologies, GIBCO BRL, Gaithersburg, Md.; catalog number 18089-011), referring to Rashtchian, A., PCR Methods Applic., 4:S83-S91(1994).

In some embodiments, an expression level of the miRNA is quantified after the amplification reaction of nucleic acids. In an example, an amplified product may be isolated on agarose gel, stained with ethidium bromide, and then detected and quantified by standard gel electrophoresis. Alternatively, the amplified product may be entirely labeled with a proper detectable marker (such as radioactive or fluorescent nucleotides), and then observed with an X-ray film or a proper excitation spectrum.

In some embodiments, an expression level of the miRNA is quantified during the amplification reaction of nucleic acids. These embodiments are also referred to as real-time amplification or quantitative amplification. Methods for quantitative amplification are disclosed in the following articles: U.S. Pat. Nos. 6,180,349, 6,180,349, and 6,033,854; Gibson et al., Genome Research (1996) 6:995-1001; DeGraves, et al., Biotechniques (2003) 34(1): 106-10, 112-5; and Deiman B, et al., Mol Biotechnol. (2002) 20(2): 163-79. The amplification is quantified based on a detectable signal, which can characterize the copy number of the template, monitored in the cycle of the amplification (such as PCR) reaction.

In some embodiments, an expression level of the miRNA may be measured by using an intercalator (such as a DNA double-strand intercalator). The intercalator can generate a detectable signal when inserted into a DNA double strand. For example, the intercalator includes SYBR GREEN™ and SYBR GOLD™. A primer or probe may or may not include a detectable marker.

In some embodiments, an expression level of the miRNA may be measured by using the primer or probe provided by this application. The primer or probe may or may not include a detectable marker.

In some embodiments, a labeled primer or probe includes a detectable marker containing a fluorophore. In some embodiments, a labeled primer or probe further includes a quencher. Such primer or probe containing both the fluorophore and quencher can be used as a self-quenching primer or probe. In a complete primer or probe, a quencher and a fluorophore are very close. Therefore, the activated fluorophore transfers energy into the quencher in the same probe through fluorescence resonance energy transfer (FRET), to emit no signal. For example, one of the fluorophore and quencher may be located at an end of the primer or probe, and the other may be located at an opposite end or optionally linked to an internal nucleotide. In another example, both the fluorophore and the quencher may be linked to the internal nucleotides with a certain distance from each other, as long as FRET can occur. The self-quenching primer or probe includes, but is not limited to, a TaqMan probe (referring to U.S. Pat. Nos. 5,210,015 and 5,538,848), a Molecular Beacon probe (referring to U.S. Pat. Nos. 5,118,801 and 5,312,728), or other stemless or linear beacon probes (referring to Livak et al., 1995, PCR Method Appl., 4:357-362; Tyagi et al, 1996, Nature Biotechnology, 14:303-308; Nazarenko et al., 1997, Nucl. Acids Res., 25:2516-2521; and U.S. Pat. Nos. 5,866,336 and 6,117,635).

The primer or probe labeled with a fluorophore and quencher can be used in the PCR with 5′-3′ exoribonucleases for “hydrolyzing” (which is referred to as TaqMan® measurement) (referring to U.S. Pat Nos. 5,210,015 and 5,487,972; Holland et al., PNAS USA (1991) 88: 7276-7280; and Lee et al, Nucleic Acids Res. (1993) 21: 3761-3766). The measurement is carried out to detect the accumulation of specific PCR products by hybridizing and cleavaging the labeled probe (the “TaqMan®” probe) during the amplification reaction. In the PCR process, the probe can be actively cleaved by the 5′-3′ exoribonuclease of the DNA polymerase when and only when the probe hybridizes with an amplified segment. The cleavage of the probe causes a corresponding increase in the fluorescence intensity of the fluorophore.

Alternatively, other types of primer or probe including a detectable marker may be used. In an embodiment, a labeled primer or probe is configured such that there will be a change in fluorescence when the primer or probe hybridizes with a target nucleic acid. For example, two probes may be designed to have a fluorophore and a quencher respectively, so that the two probes can hybridize with the target nucleic acid in a head-to-tail orientation and have a distance that allows FRET to occur between the fluorophore and the quencher after the hybridization, such as the LightCycler™ hybridization probes. In another embodiment, a labeled primer or probe is configured to emit a signal when binding to a nucleic acid or being incorporated into an elongation product, such as the Scorpions™ probe (referring to Whitcombe et al., Nature Biotechnology (1999) 17:804-807 and U.S. Pat. No. 6,326,145) and the Sunrise™ (or Amplifluor™) probe (referring to Nazarenko et al., Nuc. Acids Res. (1997) 25:2516-2521 and U.S. Pat. No. 6,117,635). In another embodiment, a labeled primer or probe is configured to have a secondary structure (such as the Lux probes™ probe). The secondary structure per se (i.e., a quencher is not necessary) leads to a decrease in signal, but the secondary structure is destroyed after hybridizing with a target nucleic acid, so that an enhanced signal can be emitted.

In the quantitative amplification reaction (such as real-time PCR), the detected signal can be quantified by a method known in the art to obtain the level of the miRNA. In some embodiments, during the amplification, the signal intensity of an intercalator, an labeled primer, or an labeled probe is directly proportional to the amount of amplified miRNA, thereby quantifing the amount of original miRNA in the sample is feasible. In some embodiments, a fluorescent signal may be monitored and calculated in each cycle of PCR amplification, and the fluorescent signal increases as the number of cycles increases. In some embodiments, a cycle threshold or a Ct value may also be calculated. The Ct value is the number of cycles required when the fluorescence reaches a predetermined value.

Hybridization-Based Method

The hybridization-based method refers to a method for detection based on nucleic acid hybridization. The hybridization-based method includes, but is not limited to, northern blotting, southern blotting, in situ hybridization, microarray analysis, microsphere technology-based hybridization, multiplex hybridization, and the like. In some embodiments, the hybridization-based method may not include nucleic acid amplification.

In some embodiments, the test nucleic acids (such as miRNA) may be isolated by, for example, gel electrophoresis, and then the isolated nucleic acids may be transferred with a membrane filter (such as a nitrocellulose membrane filter), to allow a probe to hybridize with the isolated target nucleic acid for detection (referring to Molecular Cloning: A Laboratory Manual, J. Sambrook et al., eds., 2nd edition, Cold Spring Harbor Laboratory Press, 1989, Chapter 7). The hybridization of the probe with the target nucleic acid may be detected or measured by a method known in the art. For example, after the hybridization, the membrane filter may be subjected to autoradiography on a photosensitive film. The exposed photosensitive film is subjected to densitometry scanning to measure the level of the target nucleic acid accurately. The level of the target nucleic acid may be calculated by a computer imaging system.

In some embodiments, a probe used for hybridization may include a detectable marker. For example, the test nucleic acids (such as miRNA) in a sample may hybridize with labeled probes, and the labeled probes that are unhybridized are washed away, and then the test nucleic acids in the sample can be detected.

In some embodiments, a probe used for hybridization may not include a detectable marker. For example, unlabeled probes may be immobilized on a solid support in a way that they are spatially isolated from each other and may hybridize with labeled target nucleic acid molecules.

In some embodiments, the detection based on hybridization may be carried out by using a microarray. The microarray provides a method for measuring the levels of a large number of target nucleic acid molecules simultaneously. The microarray includes a solid substrate and a plurality of nucleic acid probes immobilized on the solid substrate in a way that they are spatially isolated from each other. The nucleic acid probes may be arranged on the surface of the solid substrate in a density of up to millions of probes per square centimeter. Laser scanning may be carried out on the probes on the microarray that have hybridized with the target nucleic acid molecules (such as miRNAs) in the sample to determine the hybridization signal intensity of each of the probes on the microarray, and the signal intensity is converted into a quantitative value that represents the levels of the target nucleic acid molecules (such as miRNAs) (referring to U.S. Pat. Nos. 6,040,138, 5,800,992, 6,020,135, 6,033,860, and 6,344,316).

Sequencing-Based Method

The sequencing-based method refers to a method including nucleic acid sequencing. The sequencing-based method includes, but is not limited to, RNA sequencing, pyrosequencing, and high-throughput sequencing. The high-throughput sequencing (also referred to as second-generation sequencing) characterized by massive parallel sequencing can be used to measure the expression levels of target nucleic acids (such as miRNAs). The high-throughput sequencing includes massive parallel signature sequencing (MPSS) Polony sequencing, 454 pyrosequencing, Illumina (Solexa) sequencing, SOLiD sequencing, ion semiconductor sequencing, DNA nanoball sequencing, Helioscope™ single-molecule sequencing, single-molecule SMRT™ sequencing, Real-Time Single-Molecule (RNAP) sequencing, nanopore DNA sequencing, and the like.

The high-throughput sequencing may include sequencing-by-synthesis, sequencing-by-ligation, and ultra-deep sequencing (referring to Marguiles et al., Nature 437 (7057): 376-80 (2005)). The sequencing-by-synthesis usually uses four differently labeled nucleotides during the sequencing. A label signal of a certain labeled nucleotide incorporated into an elongated complementary strand is detected and recognized, and then the 3′ termination group and detected label on the nucleotide are removed to allow a next labeled nucleotide to be incorporated. The sequencing can be completed by repeating the foregoing steps of incorporation, detection, and recognition. More examples and specific descriptions about the sequencing-by-synthesis may refer to U.S. Pat. Nos. 7,056,676, 8,802,368, and 7,169,560. The sequencing-by-synthesis may be carried out on the surface of a solid support (a microarray or a chip) based on inverse PCR and an anchored primer. In some embodiments, because miRNA is short, an adapter sequence can be introduced onto the RNA (such as enriched RNA) in a sample or the DNA obtained by reverse transcription of the RNA, for example, an adapter sequence is ligated at the 5′ end and/or 3′ end. The adapter sequence can hybridize with a complement sequence thereof immobilized on a solid substrate or a nucleic acid microarray, so that the nucleic acid molecules containing the adapter sequence in the sample bind to the microarray, and are then amplified and sequenced by, for example, bridge PCR. This technology is used in, for example, the Illumina® sequencing platform.

For the pyrosequencing, a target nucleic acid region hybridizes with a primer by sequentially incorporating deoxyribonucleoside triphosphates corresponding to bases A, C, G, and T(U) in the presence of polymerase to elongate a new strand. Pyrophosphate is released with the incorporation of each base and converted by sulfurylase into ATP. The sulfurylase causes the synthesis of oxyluciferin and the release of visible light. Since the release of pyrophosphate is equimolar with the incorporation of bases, the visible light released is proportional to the amount of nucleotides incorporated in any step. This process may be repeated until the entire sequence is determined.

In some embodiments, a level of the miRNA is measured by whole transcriptome shotgun sequencing (RNA sequencing). A method for RNA sequencing has been disclosed (referring to Wang Z, Gerstein M and Snyder M, Nature Review Genetics (2009) 10:57-63; Maher C A et al., Nature (2009) 458:97-101; and Kukurba K & Montgomery S B, Cold Spring Harbor Protocols (2015) 2015(11): 951-969).

A sequenced target nucleic acid can be quantified by counting its copy number.

Expression Level of miRNA

An expression level of miRNA can be determined by a method known to a person skilled in the art. For example, a measured signal may be converted into an expression level of miRNA by an external standard curve method or an internal standard method.

In some embodiments, an expression level of miRNA may be normalized. For example, the expression level of miRNA may be normalized to a standard level. In some embodiments, the standard level may be a measured level of a known standard product, or may be a measured level of a certain endogenous reference in the test sample. In some embodiments, the standard level may be a collection of measured levels of a plurality of standard products. For example, the standard level may be total reads of a sequencing reaction. The standard level may be pre-determined, or may be measured simultaneously with a test sample. The normalization of the expression level of miRNA can eliminate the difference in the measured value of the expression level of miRNA between different samples due to, for example, the difference in the sample size, so that the miRNA levels between different samples or between different experiments are comparable.

In some embodiments, the expression levels of the miRNAs is normalized to an endogenous reference. The endogenous reference, also referred to as internal standard, refers to a nucleic acid molecule that is endogenously present in a test sample. In some embodiments, the endogenous reference may include a mRNA level of a conserved gene from the same sample. The conserved gene used as the endogenous reference includes actomyosin, glyceraldehyde 3-phosphate dehydrogenase (G3PDH), ribosomal RNA (such as 16s-rRNA), and the like.

In some embodiments, the endogenous reference may include one or more miRNAs from the miRNA combination. In some embodiments, the endogenous reference may be a miRNA with no significant or small difference in expression levels between subjects with bladder and urothelial carcinoma and subjects without bladder and urothelial carcinoma. In some embodiments, in the miRNA combination, miRNAs as the endogenous reference include hsa-miR-99a or hsa-miR-100, hsa-miR-143#, and the like. hsa-miR-99a and hsa-miR-100 differ by only one base in the sequence, and are similar in biological activity and function, which are expected to have similar effects in the miRNA combination of the present disclosure. It is expected that hsa-miR-99a and hsa-miR-100 are interchangeable in some combinations of the present disclosure.

In some embodiments, an expression level of the miRNA may be a relative expression level of the miRNA. The relative expression level may be calculated into a ratio by comparing an expression level of a miRNA from a miRNA combination with an expression level of another miRNA from the combination or with a sum of expression levels of two or more miRNAs from the combination. For example, a ratio may be calculated by comparing an expression level of hsa-miR-151-5p with an expression level of hsa-miR-99a to be used as a relative expression level of hsa-miR-151-5p or a relative expression level of hsa-miR-99a.

In some embodiments, a relative expression level of a miRNA may be calculated by comparing with an expression level of a certain miRNA from the same miRNA combination. For example, a relative expression level of a miRNA may be calculated by comparing with an expression level of one or more endogenous reference miRNAs (such as hsa-miR-99a). In some embodiments, a non-endogenous reference miRNA may be used for comparison, for example, the miRNA with large difference in expression levels between subjects with bladder and urothelial carcinoma and subjects without bladder and urothelial carcinoma. In some embodiments, in the miRNA combination, miRNAs may be compared with the same miRNA, or may be compared with different miRNAs, for example, hsa-miR-151-5p is compared with hsa-miR-99a, and hsa-miR-125b is compared with hsa-miR-96. A person skilled in the art may pair miRNAs in the miRNA combination according to actual conditions to calculate a relative expression level of each miRNA. For example, in this application, the miRNA combinations 11, 11a, 11b, and 11c are the same but with different relative expression levels of miRNAs calculated based on different miRNA pairs.

Therefore, in this application, the term “expression level” should be understood to include a measured expression level, a normalized expression level, and a relative expression level calculated based on miRNA pairs.

Calculation of Expression Pattern

In some embodiments, the step c) includes comparing the expression patterns between the experimental group and the control group to determine whether there is a significant difference, and further includes calculating an expression pattern of the miRNA combination, based on the expression levels of the miRNAs.

In this application, the “expression pattern” refers to a pattern comprehensively reflected by the expression levels of a plurality of miRNAs, which can reflect sample states (such as normal samples or cancer samples). Two samples may show similar expression levels on one specific miRNA, but may not show the same expression levels on a plurality of miRNAs. Different miRNAs may have different effects on sample states. For example, the overexpression of some miRNAs may be more likely to reflect a disease state of samples, or may be more likely to reflect a normal state of samples. This reflects that different miRNAs may have different decision weights in the classification of sample states. Therefore, the expression pattern may be calculated through a function or model related to the expression level of each miRNA and a decision weight of each miRNA for sample states. In some embodiments, a calculation result may be represented by a value.

In some embodiments, the function or model for calculating the expression pattern is calculated by a classification algorithm. The classification algorithm is an algorithm that classifies events with a set of variables. The classification algorithm can classify patients, subjects, or events into one category or different categories based on the parameters or data obtained therefrom. For example, patients may be classified into patients at a high risk of disease or patients at a low risk of disease, or may be classified into patients requiring treatment or patients without requiring treatment. In some embodiments, the calculated expression pattern can reflect categories of sample classification. For example, the expression pattern may be calculated into a decision score that helps the sample classification.

In some embodiments, the classification algorithm is selected from support vector machines, linear discriminant analysis, pattern classification (referring to Duda et al., Pattern Classification, 2nd ed., John Wiley, New York 2001), logistic regression, naive Bayes classification, perceptron classification, quadratic classification, k-nearest neighbors, boosting, decision tree (referring to Hsatie et al., The Elements of Statistical Learning, Springer, New York 2001), random forest (referring to Breiman, RandomForests, Machine Learning 45:5 2001), neural network (referring to Bishop, Neural Networks for Pattern Recognition, Clarendon Press, Oxford 1995), PAM (referring to Tibshirani et al., 2002, Proc. Natl. Acad. Sci. USA 99:6567-6572), SIMCA (referring to Wold, 1976, Pattern Recogn. 8:127-139), and learning vector quantization. In some embodiments, the classification algorithm is support vector machines. In some embodiments, softwares providing the classification algorithm may be used. Some of such softwares are open source and publicly available, for example, the R software (https://www.r-project.org/). A person skilled in the art may select a suitable program package in the R software, and select a suitable classification algorithm or function in the program package to use in the method of the present disclosure.

In some embodiments, the classification algorithm may be subjected to at least one training of a positive training data set and a negative training data set to provide one or more decision weights of the function or model for calculating the expression pattern. It may be understood that the function or model for calculating the expression pattern may be different based on different classification algorithms. A person skilled in the art may determine a proper function or model based on a selected classification algorithm. The positive training data set includes the expression level of each miRNA in the miRNA combination from a plurality of urine samples of subjects with bladder and urothelial carcinoma, and the negative training data set includes the expression level of each miRNA in the miRNA combination from a plurality of urine samples of subjects without bladder and urothelial carcinoma.

Data of the expression levels, before used in the algorithm, may be appropriately transferred or preprocessed, such as standardization. During the training, the expression level of each miRNA in the miRNA combination from each known sample and a known classification label (such as with bladder and urothelial carcinoma or without bladder and urothelial carcinoma) are used to train the classification algorithm. The classification algorithm may divide the expression levels of miRNAs in the positive training data set and the negative training data set into two mutually exclusive parts corresponding to different clinical classifications. For example, one part corresponds to the expression pattern of suffering from bladder and urothelial carcinoma or being at a high risk of bladder and urothelial carcinoma, and the other part corresponds to the expression pattern of not suffering from bladder and urothelial carcinoma. A predicted intensity of the expression level of each miRNA may be obtained by various linear classification methods, including but not limited to, partial least squares (PLS, (Nguyen et al., 2002, Bioinformatics 18(2002)39-50)) or SVM (Schölkopf et al., Learning with Kernels, MIT Press, Cambridge 2002). In some embodiments, a weighted sum of the predicted intensity of the expression level of each miRNA in a miRNA combination from each sample is calculated. In some embodiments, data used for calculating the weighted sum may be transformed into non-linear data. The non-linear transformation may include the increase of data dimensionality. The non-linear transformation and the weighted sum may be carried out implicitly, for example, by using a kernel function (Schölkopf et al., Learning with Kernels, MIT Press, Cambridge 2002). A higher weighted sum of the predicted intensity indicates a predicted result is closer to a positive result (for example, of suffering from bladder and urothelial carcinoma or being at a high risk of bladder and urothelial carcinoma). A lower sum of the predicted intensity indicates a predicted result is closer to a negative result (for example, of not suffering from bladder and urothelial carcinoma or being at a low risk of bladder and urothelial carcinoma). Parameters, that is, decision weights, of functions related to the classification algorithm can be optimized based on known information from the positive training data set and the negative training data set, so as to achieve optimal classification prediction on unclassified samples. In this step of training, the classification algorithm is trained or parameterized.

In some embodiments, the training includes training with the positive training data set and the negative training data set to provide one or more decision weights of the function or model for calculating a positive expression pattern and one or more decision weights of the function or model for calculating a negative expression pattern. The positive expression pattern refers to an expression pattern indicating a positive prediction result, and the negative expression pattern refers to an expression pattern indicating a negative prediction result. In some embodiments, the expression pattern is calculated into a weighted sum of the predicted intensity. In some embodiments, the expression pattern is a score between 0 and 1. For example, if the expression pattern is closer to 0, a prediction result is closer to a negative result (that is, not suffering from bladder and urothelial carcinoma); and if the expression pattern is closer to 1, a prediction result is closer to a positive result (that is, suffering from bladder and urothelial carcinoma or being at a high risk of bladder and urothelial carcinoma).

In some embodiments, the training further includes determining a threshold based on a score of the positive expression pattern and a score of the negative expression pattern, wherein the threshold is able to distinguish the positive expression pattern from the negative expression pattern. In some embodiments, the threshold may be pre-calculated and determined according to historical data, or may be determined or adjusted according to other data or requirements. In some embodiments, a predetermined threshold may be set according to a required sensitivity and specificity. For example, if a higher sensitivity and a lower specificity are required, a lower threshold may be selected; and conversely, if a higher specificity and a lower sensitivity are required, a higher threshold may be selected.

In some embodiments, an expression pattern of a test urine sample is calculated by a trained classification algorithm. For example, the expression pattern may be a weighted sum of the predicted intensity of the expression level of each miRNA in a miRNA combination from the test urine sample.

In some embodiments, the expression pattern of the test urine sample may be compared with the positive expression pattern and/or the negative expression pattern obtained from training data sets. The comparison is carried out in a proper similar way, for example, but not limited to, the Euclidean distance (Duda et al., Pattern Classification, 2nd ed., John Wiley, New York 2001), the correlation coefficient (Van't Veer, et al., 2002, Nature 415:530), or the like. Then, the test urine sample may be assigned to a group with the closest expression pattern or a group with the highest quantity of expression patterns in the vicinity.

In some embodiments, the method further includes comparing a score of the expression pattern calculated in the test urine sample with the threshold to assess whether the test subject suffers from bladder and urothelial carcinoma or is at a high risk of bladder and urothelial carcinoma. In some embodiments, when the expression pattern is a score between 0 and 1, the threshold may be set to a proper value between 0.2 and 0.8, depending on specific requirements for sensitivity and specificity. In some embodiments, the threshold is set to 0.2, 0.3, 0.4, 0.5, 0.6, 0.7, or 0.8. In some embodiments, the threshold is set to 0.4. If the score of the expression pattern calculated in the test urine sample is greater than the threshold, the test subject is assessed as suffering from bladder and urothelial carcinoma or being at a high risk of bladder and urothelial carcinoma.

In some embodiments, the method provided by this application further includes administering a bladder and urothelial carcinoma therapy to the test subject assessed as suffering from bladder and urothelial carcinoma or being at a high risk of bladder and urothelial carcinoma in the step c). In some embodiments, the bladder and urothelial carcinoma therapy includes radiotherapy, surgery, anti-cancer drug therapy, or any combination thereof. The anti-cancer drug therapy may use any anti-cancer drugs effective for bladder and urothelial carcinoma. The anti-cancer drugs may include compounds for targeted therapy, compounds for conventional untargeted chemotherapy, or drugs for immunotherapy.

The anti-cancer drugs include, but are not limited to, alkylating agents, antimetabolites, alkaloids, cytotoxic/anti-cancer antibiotics, topoisomerase inhibitors, tubulin inhibitors, proteins, antibodies, kinase inhibitors (such as serine-threonine kinase antagonists and tyrosine kinase antagonists), fibroblast growth factor receptor inhibitors, transcription inhibitors, G protein-coupled receptor antagonists, growth factor antagonists, immune checkpoint modulators, and the like. The immune checkpoint includes: PD-1, PD-L1, PD-L2, CTLA-4, TIM-3, LAG3, CD160, 2B4, TGFβ, VISTA, BTLA, TIGIT, LAIR1, OX40, CD2, CD27, CDS, ICAM-1, NKG2C, SLAMF7, NKp80, B7-H3, LFA-1, 1COS, 4-1BB, GITR, CD30, CD40, BAFFR, HVEM, CD7, LIGHT, or CD83 ligand. The immune checkpoint modulator can modulate immune checkpoint molecules to restore the anti-tumor activity of T cells and/or block the inhibition of T cells.

For example, the anti-cancer drugs may include erdafitinib, thiotepa, Bacillus Calmette-Guérin vaccine, gemcitabine, cisplatin, carboplatin, oxaliplatin, doxorubicin, epirubicin, idarubicin, valrubicin, paclitaxel, imatinib, dactinomycin, all-trans retinoic acid, azacitidine, azathioprine, bleomycin, bortezomib, capecitabine, nitrogen mustard, chlorambucil, cyclophosphamide, cytarabine, daunorubicin, docetaxel, doxorubicin, epothilone, etoposide, teniposide, fluorouracil, tioguanine, deoxyuridine, mercaptopurine, hydroxyurea, irinotecan, methotrexate, mitoxantrone, pemetrexed, topotecan, vemurafenib, vinblastine, vincristine, vindesine, vinorelbine, hydroxycamptothecin, PD-1 antibody (such as pembrolizumab and nivolumab), CTLA-4 antibody (such as ipilimumab and tremelimumab), PD-L1 antibody (such as atezolizumab, durvalumab, and avelumab), or the like.

Nucleic Acid Chip

According to another aspect, the present disclosure further provides a miRNA detecting chip (also referred to as a detection microarray), including: the isolated oligonucleotides provided by this application immobilized on a solid support. In the method provided by this application, to obtain expression levels of a plurality of miRNAs in the miRNA combination, a plurality of different miRNAs in a sample may be detected simultaneously. The miRNA detecting chip functions in this case.

In this application, the “chip” refers to a nucleic acid microarray, that is, an array with a plurality of addressable locations (that is, locations characterized by distinctive and accessible addresses), and each addressable location includes one oligonucleotide with a specific sequence that is connected to it. Each microarray may have dozens, hundreds, or even thousands of addressable locations and oligonucleotides connected thereto. The oligonucleotide array may be divided into a plurality of subarrays according to requirements. In some embodiments, oligonucleotides on the miRNA chip are immobilized on the solid support at a specific density (for example, at least 100/cm²) and in a way that they are spatially isolated from each other. For a test miRNA or different segments of the test miRNA, there may be different nucleic acid probes with sequences that may be partially overlapped on the nucleic acid microarray.

The solid support suitable for the miRNA detecting chip may be made of various common materials in the field of gene chips, for example, but not limited to, a nylon membrane, an active group-modified glass slide or silicon wafer, an unmodified glass slide, a plastic piece, microspheres, gel, a polymer surface, and an optical fiber, a glass fiber, or a fiber with any proper matrix (referring to U.S. Pat. Nos. 5,770,358, 5,789,162, 5,708,153, 6,040,193, and 5,800,992).

The miRNA detecting chip may be prepared by a conventional method for manufacturing biochips known in the art. For example, in case of a modified glass slide or silicon wafer as the solid support, and presence of an amino-modified poly dT strand at 5′ end of a probe, the miRNA chip according to this application can be obtained by: preparing a solution of the oligonucleotide probes, spotting the solution on the modified glass slide or silicon wafer in a predetermined sequence or array by using a spotter, and fixing. This method may refer to U.S. Pat. Nos. 6,329,209, 6,365,418, 6,406,921, 6,475,808, and 6,475,809. If the nucleic acid does not include amino modification, the preparation method may refer to: Gene Diagnosis Technology-Non-Radioactive Operation Manual, edited by Wang Shenwu; J. L. erisi, V. R. Iyer, P.O. BROWN. Exploring the metabolic and genetic control of gene expression on a genomic scale. Science, 278:680 (1997); and Biochips, edited by Ma Liren and Jiang Zhonghua, Beijing: Chemical Industry Press, 2000, 1-130. Although a flat chip surface is generally used, the chip can be built on one or even more surfaces of almost any shape.

There are several advantages to using the miRNA chip to detect the expression levels of miRNAs. First, the overall expression of a plurality of miRNAs in the same sample can be recognized at the same time point. Second, the expression of mature molecules and precursor molecules can be recognized by properly designing the oligonucleotide probe. Third, compared with the Northern blot analysis, the chip requires a small amount of RNA, that is, a reproducible result can be provided by using only 2.5 μg of RNA in total. The miRNA chip can help gene expression profiling to analyze the expression pattern and expression levels of miRNAs. Different miRNA features may be related to established disease markers, or may be directly related to disease states.

The miRNA detecting chip or the oligonucleotides on the nucleic acid microarray provided by this application may include the oligonucleotide probes (such as single-stranded nucleic acid probes) or the oligonucleotide primers provided by this application. The primer or probe may hybridize with a test miRNA in a urine sample of a test subject to allow the amplification and/or detection of the test miRNA. In some embodiments, the miRNA chip provided by this application may include two different oligonucleotide probes for each miRNA, one having specificity for miRNAs with active mature sequences, and the other having specificity for miRNA precursors. In some embodiments, the miRNA chip provided by this application may also include some controls, such as one or more mouse sequences that differ from human homologous genes by only a few bases. The mouse sequences may be used as a control for stringent hybridization conditions. The miRNAs of two species may be printed on the chip to provide an internal, relatively stable, and positive control for specific hybridization. The chip may also include one or more proper controls for non-specific hybridization. For this reason, a sequence with a large difference from known miRNA sequences may be selected as a control for non-specific hybridization. In some embodiments, the miRNA chip provided by this application also includes a positive control, such as some synthesized miRNAs or homologous endogenous small RNAs like hsa-RNU48 and hsa-7SL-scRNA.

In some embodiments, the oligonucleotide probe further includes a detectable marker.

In some embodiments, the miRNA detecting chip or the oligonucleotides on the nucleic acid microarray may further include a connection region connected to the solid support.

In some embodiments, the miRNA chip may pre-hybridize with a pre-hybridization buffer before RNA in a test sample hybridizes with the miRNA chip. In the present disclosure, the solid-phase hybridization between RNA and the miRNA chip is carried out by a conventional method in the art. A person of ordinary skill in the art can easily determine the optimal concentrations of the related buffer, probe, and sample, the optimal pre-hybridization temperature, the optimal hybridization temperature, and the optimal time based on experience. Alternatively, the solid-phase hybridization may be carried out by the method with reference to Molecular Cloning: A Laboratory Manual. Then, test information is obtained according to the location and intensity of a label signal on the miRNA chip. If an amplified product is labeled with a fluorophore, the test information may be directly obtained by using a fluorescence detection device (such as a laser confocal scanner Scanarray 3000).

According to another aspect, the miRNA detecting chip provided by the present disclosure may be used to prepare a diagnostic kit for diagnosing bladder and urothelial carcinoma.

This application further provides use of a diagnostic kit for diagnosing bladder and urothelial carcinoma prepared with the miRNA detecting chip provided by this application.

Detection Kit

According to an aspect, this application provides a kit for detecting a miRNA combination, including the isolated oligonucleotides provided by this application and/or the miRNA detecting chip provided by this application.

In some embodiments, the kit provided by this application further includes a positive control. For example, the kit may also include the miRNA combination provided by this application or the oligonucleotides coding the miRNA combination.

In some embodiments, the kit provided by this application further includes a negative control. For example, the kit may also include one or more proper controls for non-specific hybridization. The sequence of the negative control may be a sequence that does not have significant homology with the known test miRNA combination.

In some embodiments, the kit provided by this application further includes a control with hybridization conditions. For example, the kit may further include one or more mouse sequences that differ from human homologous miRNA genes by only a few bases. The mouse sequences may be used as a control for stringent hybridization conditions.

In some embodiments, the kit provided by this application further includes a marker for labeling RNA samples and a substrate corresponding to the marker.

In some embodiments, the kit provided by this application further includes one or more reagents selected from the following group: reagents for reverse transcription, reagents for RNA enrichment, reagents for nucleic acid amplification (such as an amplification solution), reagents for hybridization (such as a hybridization solution), reagents for color development (such as a color developing reagent), reagents for cell lysis, reagents for protecting RNA from degradation, reagents for DNA amplification, and reagents for detecting DNA double-strand formation.

The reagents for detecting DNA double-strand formation may include an intercalator (such as EvaGreen or SYBRGreen), a primer, a fluorescent probe, and the like.

In some embodiments, the kit provided by this application also includes a control reference, such as a primer or probe that can hybridize with an endogenous reference nucleic acid sequence or a reference miRNA of known composition and/or content. In some embodiments, the isolated oligonucleotides further include a reference oligonucleotide that can hybridize with an endogenous reference, such as a reference oligonucleotide primer or a reference oligonucleotide probe.

In some embodiments, in the kit provided by this application, various reagents may be contained in different containers (such as small tubes and small bottles), or at least some of the reagents may be contained in a container as a mixture (such as a reaction mixture for PCR).

In some embodiments, the kit provided by this application also includes a device for sampling the urine sample.

In addition, in some embodiments, the kit provided by this application may also include instructions.

In some embodiments, the kit provided by this application further includes a non-transient computer-readable medium, the non-transient computer-readable medium including a computer-executable instruction of calculating an expression pattern of the miRNA combination based on the expression levels of the miRNAs. In some embodiments, the computer-executable instruction includes a classification algorithm. In some embodiments, the classification algorithm has been subjected to training with a positive training data set and a negative training data set, the positive training data set includes the expression level of each miRNA in the miRNA combination from a plurality of urine samples of subjects with bladder and urothelial carcinoma, and the negative training data set includes the expression level of each miRNA in the miRNA combination from a plurality of urine samples of subjects without bladder and urothelial carcinoma.

According to another aspect, this application further provides use of a diagnostic kit for assessing whether a subject suffers from bladder and urothelial carcinoma or is at a high risk of bladder and urothelial carcinoma prepared with the miRNA detection reagents and/or the miRNA detecting chip.

Method of Screening Drug Candidate

This application further provides a method of screening a drug candidate for treating bladder and urothelial carcinoma based on the miRNA combination provided by this application. In some embodiments, drug candidates for the method in this application may include proteins, peptides, nucleic acids such as DNA and RNA, and small molecules such as organic or inorganic molecules with a molecular weight less than 50 kd. The drug candidates may be endogenous physiological compounds, or natural or synthetic compounds. In some embodiments, proper drug candidates include, but are not limited to, a transcription inhibitor, a G protein-coupled receptor antagonist, a growth factor antagonist, a serine-threonine kinase antagonist, and a tyrosine kinase antagonist. The drug candidate may alternatively be a combination of the foregoing drugs. It can be determined whether a drug candidate has a potential therapeutic effect on bladder and urothelial carcinoma by comparing the changes in the expression levels of the miRNA combination in cancer cells before and after the treatment with the drug candidate.

In some embodiments, the method includes measuring an expression level of each miRNA in the miRNA combination from bladder and urothelial carcinoma cells in an experimental group that are treated with a drug candidate to provide an expression level of the experimental group. The expression level of the experimental group may be calculated to obtain an expression pattern of the miRNA combination in the experimental group as the expression pattern of the experimental group.

In some embodiments, the method also includes measuring an expression level of each miRNA in the miRNA combination from bladder and urothelial carcinoma cells in a control group that are not treated with a drug candidate to provide an expression level of the control group. The expression level of the control group may be calculated to obtain an expression pattern of the miRNA combination in the control group as the expression pattern of the control group.

The expression pattern of the experimental group and the expression pattern of the control group may be calculated by, for example, a classification algorithm. In some embodiments, the expression pattern of the experimental group and the expression pattern of the control group each may be calculated into a score between 0 and 1. If a score of the experimental group is significantly lower than a score of the control group, the drug candidate has a potential therapeutic effect on bladder and urothelial carcinoma.

In some embodiments, the bladder and urothelial carcinoma cells of the experimental group and the control group have the same source. For example, the bladder and urothelial carcinoma cells of the experimental group and the control group are all from a biological sample of the same subject or are all from a bladder and urothelial carcinoma cell line. The difference between the cells of the experimental group and the control group may only be whether they are treated with a drug candidate.

In some embodiments, the method of screening a drug candidate provided by this application further includes: extracting RNAs from the bladder and urothelial carcinoma cells of the experimental group and the control group.

In some embodiments, the expression levels of miRNAs may be measured by an amplification-based method, a hybridization-based method, or a sequencing-based method. In some embodiments, the expression levels of miRNAs are measured by using the miRNA chip.

In some embodiments, the method of screening a drug candidate further includes measuring the expression level of each miRNA in the miRNA combination from bladder and urothelial carcinoma cells in a positive group that are treated with a positive drug to provide the expression level of the positive group. The expression level of the positive group may be calculated to obtain an expression pattern of the miRNA combination in the positive group as the expression pattern of the positive group.

In some embodiments, the expression pattern of the positive group may be calculated into a score between 0 and 1. If a score of the experimental group is significantly lower than a score of the control group, and/or is close to a score of the positive group, the drug candidate has a potential therapeutic effect on bladder and urothelial carcinoma.

The foregoing features mentioned in the present disclosure or the features mentioned in the embodiments may be combined arbitrarily. All the features disclosed in the specification of this application may be used in combination with any composition form, and each feature disclosed in the specification may be replaced by any alternative feature that provides the same, equivalent, or similar purpose. Therefore, unless otherwise specified, the disclosed features are only general examples of equivalent or similar features.

The following further describes the present disclosure with reference to specific examples. It should be understood that these examples are merely intended to describe the present disclosure rather than to limit the scope of the present disclosure. The experimental methods without specific conditions described in the following examples are usually based on conventional conditions, such as conditions described in Molecular Cloning: A Laboratory Manual, Sambrook et al. (New York: Cold Spring Harbor Laboratory Press, 1989), or conditions recommended by the manufacturer. Unless otherwise specified, the percentage and the parts are the percentage by weight and the parts by weight respectively.

Example Summary

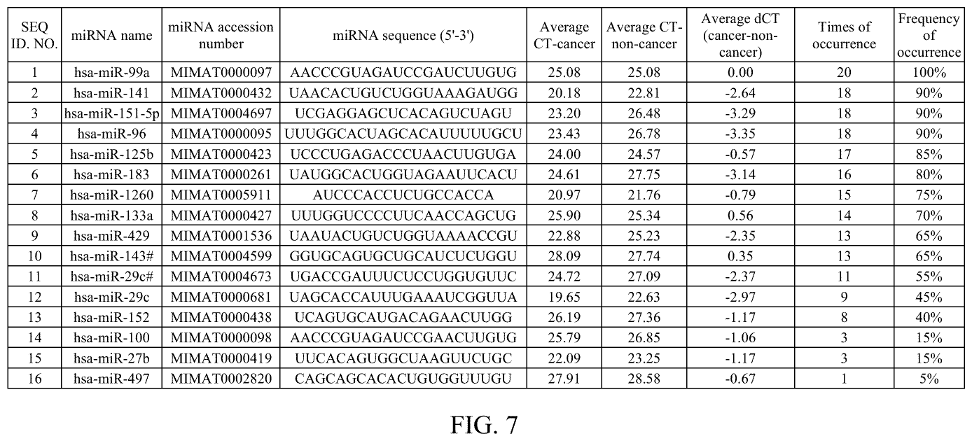

16 miRNAs were first screened out through a large amount of screenings as shown in FIG. 7. It was proved through experiments that different combinations of these miRNAs can effectively distinguish urine samples of bladder and urothelial carcinoma from urine samples of non-bladder and urothelial carcinoma.

Specifically, in this study, the expression of miRNA combinations in urine samples of bladder and urothelial carcinoma and urine samples of non-bladder and urothelial carcinoma is detected and analyzed, and a miRNA fingerprint for distinguishing the urine samples of bladder and urothelial carcinoma from the urine samples of non-bladder and urothelial carcinoma is screened out by bioinformatics.

miRNA may be detected by qPCR of miRNAs. In the previous miRNA detection, a 384-well qPCR assay matrix was built. In a 384-well PCR reaction plate, 384 qPCR reactions are carried out with 380 miRNAs with expression levels related to cancer, 2 endogenous controls, 1 exogenous control, and 1 blank control. In the present disclosure, a quantitative RT-PCR detection method based on the SYBR-GREEN fluorescence is used (referring to “DETECTION METHOD OF MICRO-RNA WITH HIGH SPECIFICITY” (ZL201110054104.7, U.S. Pat. No. 9,290,801 B2), Wang L et al., PLOS ONE. 2014, 9 (5), e96472). First, the expression of the 380 miRNAs in a urine sample of bladder and urothelial carcinoma and in a urine sample of non-bladder and urothelial carcinoma was detected and analyzed by using the 384-well matrix. 125 miRNAs with changes in the expression levels related to bladder and urothelial carcinoma were screened out. Then, the expression of the 125 miRNAs in a plurality of urine samples of patients with bladder and urothelial carcinoma and of patients without bladder and urothelial carcinoma was detected and analyzed to determine a miRNA expression fingerprint specific to bladder and urothelial carcinoma, which can effectively distinguish urine samples of bladder and urothelial carcinoma from urine samples of non-bladder and urothelial carcinoma. A detection result of the blind experiment shows that: the disclosed miRNA fingerprints can accurately and effectively distinguish urine samples of bladder and urothelial carcinoma from urine samples of non-bladder and urothelial carcinoma.

### EXAMPLE 1

### Sample Collecting and Processing

Samples in this study were human urine samples collected from a hospital. All the samples were collected with the consent of relevant subjects and approved by the ethics committee of the hospital. The pathological diagnosis results were confirmed by two or more pathologists. The clinical data of patients includes age of onset, gender, tumor type, tumor grade, etc.

A urine sample was processed within 2 h after collection. If it takes a long time, a urine sample may be stored at 4° C. for 8 h. A urine sample was centrifuged at 20° C. at 1200 g for 15 min. Then, a supernatant was completely removed, and 1 mL of DPBS (Sigma) buffer was added into the remaining precipitate to form a suspension. The suspension was transferred into a 1.5 mL centrifuge tube to be centrifuged at 20° C. at 2000 g for 5 min. Then, a supernatant was completely removed, and the remaining precipitate was stored at −80° C.

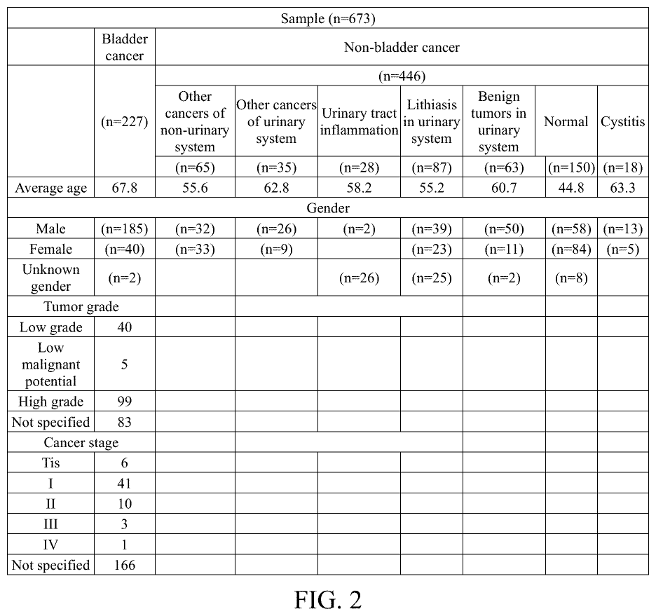

In this experiment, a total of 673 urine samples were collected and divided into a bladder and urothelial carcinoma group and a non-bladder and urothelial carcinoma control group. The bladder and urothelial carcinoma group included 227 urine samples. The bladder and urothelial carcinoma group included bladder cancer and other urothelial carcinomas such as renal pelvic carcinoma, ureter cancer, and urinary outflow tract cancer. The non-bladder and urothelial carcinoma control group included 446 urine samples. The non-bladder and urothelial carcinoma control group included a normal control, urinary tract inflammation, lithiasis in urinary system, benign tumors in urinary system, other cancers of urinary system (such as prostate cancer), non-urinary system cancer (such as liver cancer, stomach cancer, and lung cancer), and cystitis. The information of the collected samples is shown in FIG. 2.

### EXAMPLE 2

### Total RNA Extraction

The urine precipitate stored at −80° C. was taken out and placed on ice to dissolve the urine precipitate. Total RNA was extracted with reference to instructions of the miRNeasy mini kit (Qiagen, product No. 217004).

The mass was detected by electrophoresis. OD260 nm and OD280 nm were measured by ultraviolet spectroscopy, and the RNA concentration was then calculated (1 OD260 nm=40 ng/μL RNA). The total RNA was stored at −80° C.

### EXAMPLE 3

### PolyA Tailing and Reverse Transcription

The total RNA extracted in the example 2 was diluted with a 0.1× RNA storage buffer (purchased from Ambion) containing 0.1% Tween-20 (Sigma) to 4 ng/μL.

miRNA was polyA-tailed and then reverse-transcribed into cDNA by using the Sharpvue® miRNA cDNA synthesis kit (product No. 9000004) produced by Xiangqiong Biotechnology Co., Ltd. (For the specific method, refer to “DETECTION METHOD OF SMALL RNA WITH HIGH SPECIFICITY” (ZL201110054104.7, U.S. Pat. No. 9,290,801 B2)). The specific steps were as follows: A 200 μL PCR tube was placed on ice. 20 μL of total RNA with a concentration of 4 ng/μL, 10 μL of Sharpvue® miRNA cDNA synthesis reaction solution I (product No. 9000005) produced by Xiangqiong Biotechnology Co., Ltd., 3.33 μL of Sharpvue® miRNA cDNA synthesis reaction solution II (product No. 9000006) produced by Xiangqiong Biotechnology Co., Ltd., and 16.67 μL of nuclease-free ultrapure water were added in the PCR tube to a total volume of 50 μL. The total RNA mass was 80 ng. After gently mixed, the mixture was centrifuged at 1000 rpm for 10 s, and then put into the ABI 9700 PCR machine to undergo reverse transcription with a reaction procedure: at 37° C. for 60 min, at 95° C. for 10 min, and keeping at 4° C.

The PCR tube was taken out to be stored at −20° C. after shaking and centrifugation, or to be directly used for qPCR reaction.

### EXAMPLE 4

### Fluorescence Quantitative PCR Detection

30.72 μL of reverse transcription product obtained in the example 3, 768 μL of fluorescence quantitative PCR enzyme reaction solution (product No. 9000008, Sharpvue® 2× Universal qPCR Master Mix High Rox) produced by Shengyuan company, and 276.5 μL of nuclease-free ultrapure water (product No. 9000015) were added into a 1.5 mL centrifuge tube and then gently mixed.

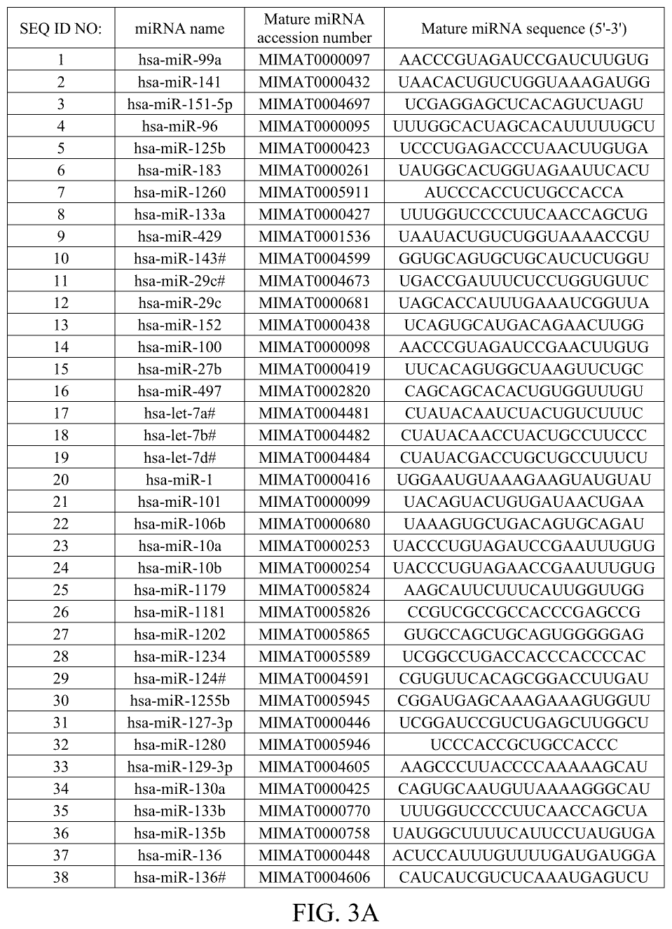

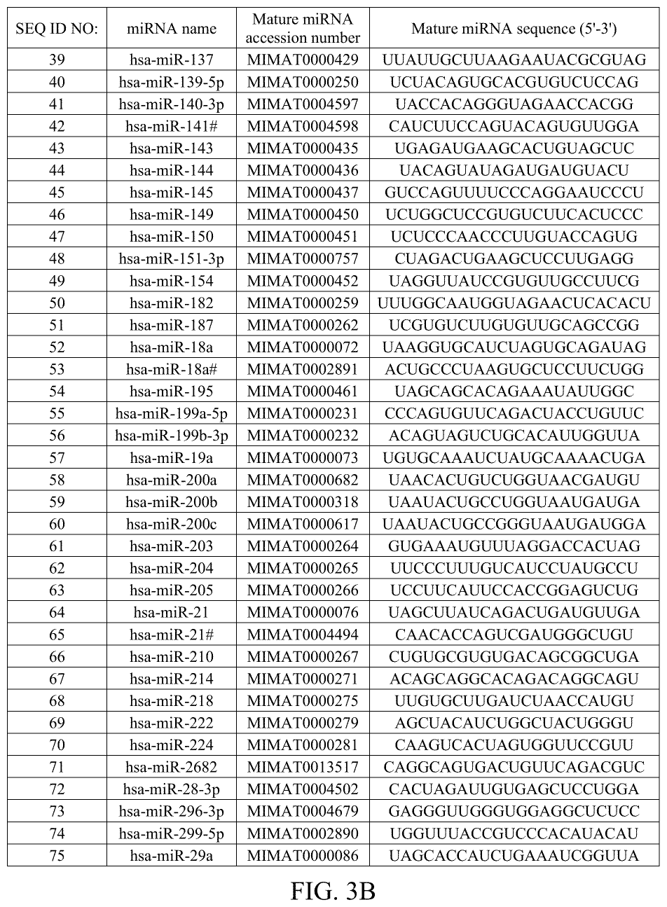

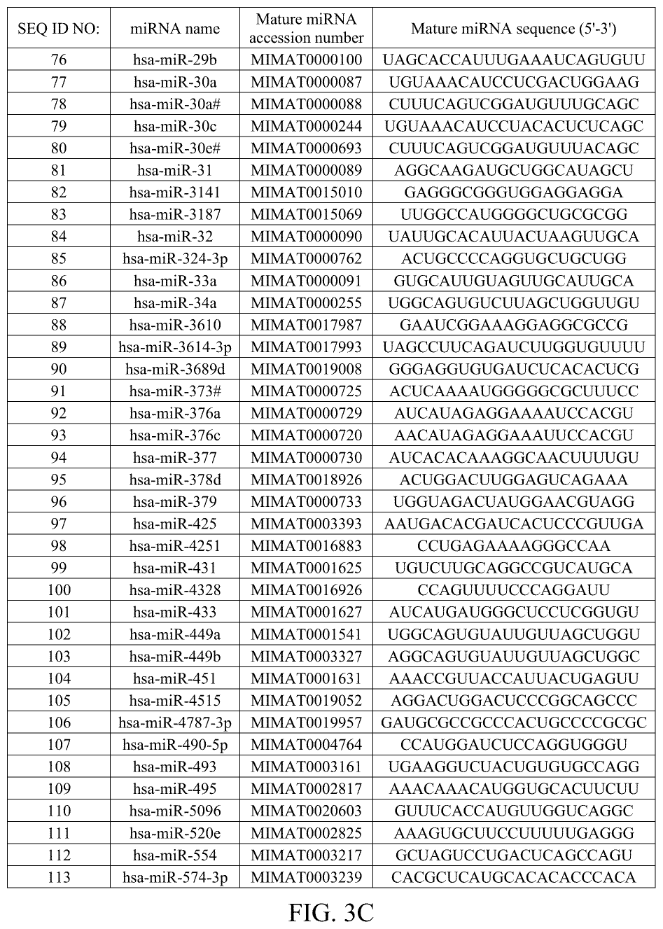

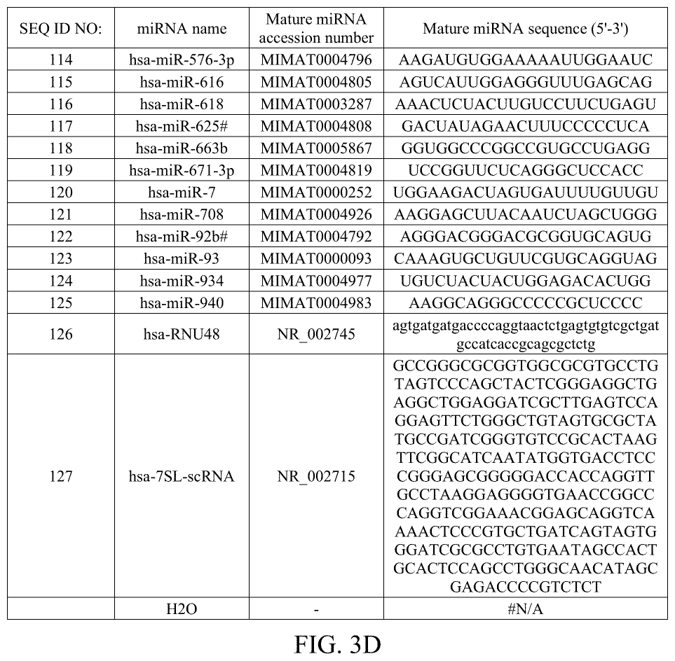

A small RNA reaction plate chip produced by Xiangqiong Biotechnology Co., Ltd. was taken out of a refrigerator at −20° C. The chip is a 384-well qPCR reaction plate including three 128-well matrices, and each matrix includes 125 wells for small RNA reaction solution, 2 wells for endogenous control, and 1 well for blank control (as shown in FIG. 3A to FIG. 3D, Sharpvue™ Bladder cancer miRNA Array, product No. 1000030). The chip was unpacked after returning to room temperature, and then placed on a centrifuge (Thermo, ST16R, rotor model: M-20) for centrifugation at 2000 g for 5 min. A sealing film was carefully removed.

The mixture obtained in the foregoing step was added into the small RNA reaction plate chip produced by Xiangqiong Biotechnology Co., Ltd. by 7 μL per well. After the addition was completed, whether the volume of liquid in each well is uniform was checked. Each small RNA reaction plate chip can be used to detect three samples each time. The detection of each sample includes 125 small RNA reactions, 2 endogenous control reactions, and 1 blank control.

The chip was sealed with a quantitative sealing film (ABI, 4711971) and then mixed upside down, and then centrifuged at 1000 g at room temperature for 2 min.

The chip was placed in a quantitative PCR machine (ABI, 7900Ht Fast) for quantitative PCR with a procedure: at 95° C. for 10 min, at 96° C. for 5 s, and at 58° C. for 1 min, for 3 cycles; then at 96° C. for 5 s, and at 60° C. for 30 s, for 37 cycles, to obtain a melting curve. The report fluorescence was set to SYBR, and the reference fluorescence was set to Rox. Data was collected for bioinformatics analysis.

### EXAMPLE 5

### Computational Analysis of miRNA Biostatistics Data

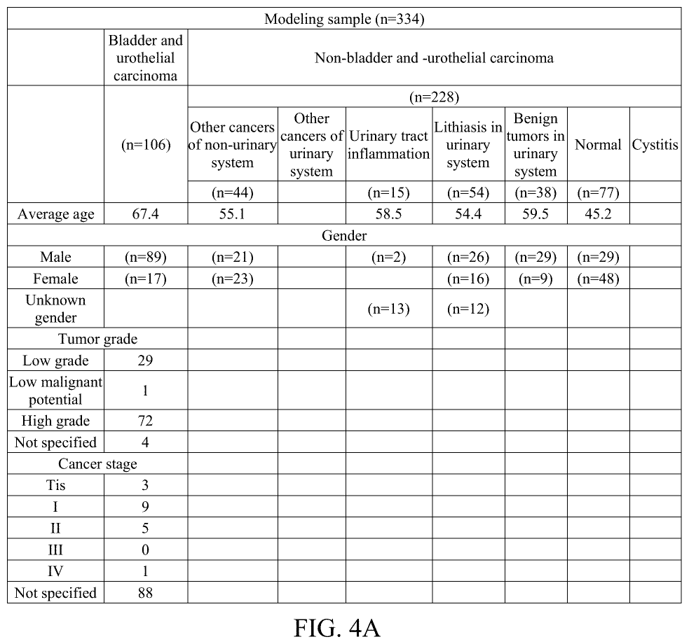

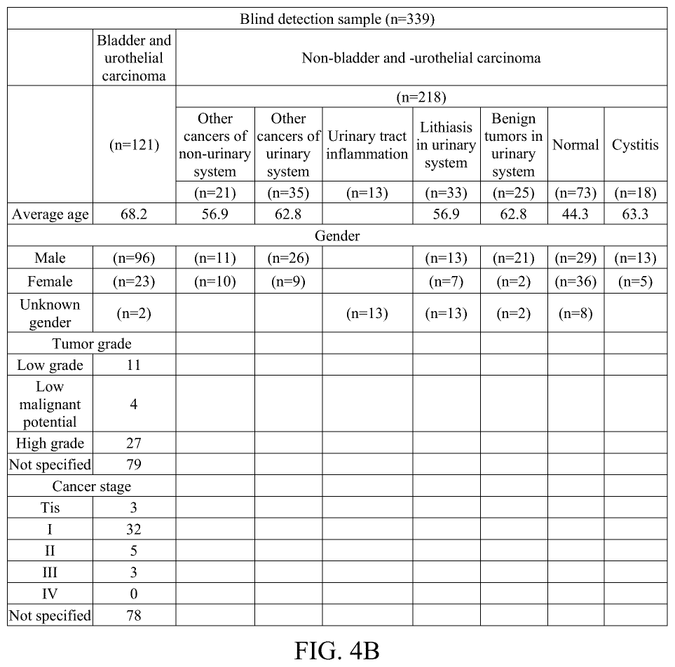

There were a total of 673 urine samples including 227 urine samples of bladder and urothelial carcinoma and 446 urine samples of non-bladder and urothelial carcinoma. The urine samples of bladder and urothelial carcinoma included bladder cancer and other urothelial carcinomas such as renal pelvic carcinoma, ureter cancer, and urinary outflow tract cancer. The urine samples of non-bladder and urothelial carcinoma control included a normal control, urinary tract inflammation, lithiasis in urinary system, benign tumors in urinary system, other cancers of urinary system (such as prostate cancer), non-urinary system cancer (such as liver cancer, stomach cancer, and lung cancer), and cystitis. The specific information is shown in FIG. 2. The 673 urine samples were divided into two parts. The first part was 334 modeling samples including 106 samples of bladder and urothelial carcinoma and 228 samples of non-bladder and urothelial carcinoma. The information about the samples is shown in FIG. 4A. The second part was 339 blind detection samples (without knowing the information of the samples by data analysts) including 121 samples of bladder and urothelial carcinoma and 218 samples of non-bladder and urothelial carcinoma. The information about the samples is shown in FIG. 4B.

125 small RNAs, 2 endogenous controls, and 1 blank control were detected by fluorescence quantitative PCR (as shown in FIG. 3A to FIG. 3D). The expression data (Ct value) of the 125 small RNAs and 2 endogenous controls was obtained through the detection. The Ct value greater than 32 was set to 32. In the analysis, 334 samples with known information were used as a training set for modeling, and it was determined whether the 339 blind samples were benign or cancer according to modeling results. The model prediction result of the blind detection samples was checked with the clinical information and pathological information provided by the hospital, and the specificity, sensitivity, and accuracy predicted for the blind detection samples were respectively counted. The software used for analysis was the R language (https://www.r-project.org/), specifically was the software package e1071 (https://cran.r-project.org/web/packages/e1071/index.html).

The SVM was selected for modeling. The specific method was as follows:

1) First, miRNAs with a low expression levels were screened out and removed. All single miRNAs and paired miRNAs were listed as marker candidates from the remaining miRNAs with high expression levels (taking the difference of the Ct value for the paired miRNAs).

2) The single miRNAs or paired miRNAs that can best distinguish a cancer group from a non-cancer group were selected through Mann-Whitney U test to be markers.

3) Any markers within 12 were selected from the markers selected in the step 2) to list all possible marker combinations. Then, 20 marker combinations of optimal models were determined according to the accuracy and area under the curve (AUC) value obtained by cross-validation.

4) Finally, a blind sample was predicted by using the 20 optimal models to determine whether it pertains to the cancer group or the non-cancer group.

The miRNA was named based on the miRNA database of miRBase Version 21.

### EXAMPLE 6

### Verification of Specificity and Sensitivity for Diagnosis on the Selected miRNA Markers

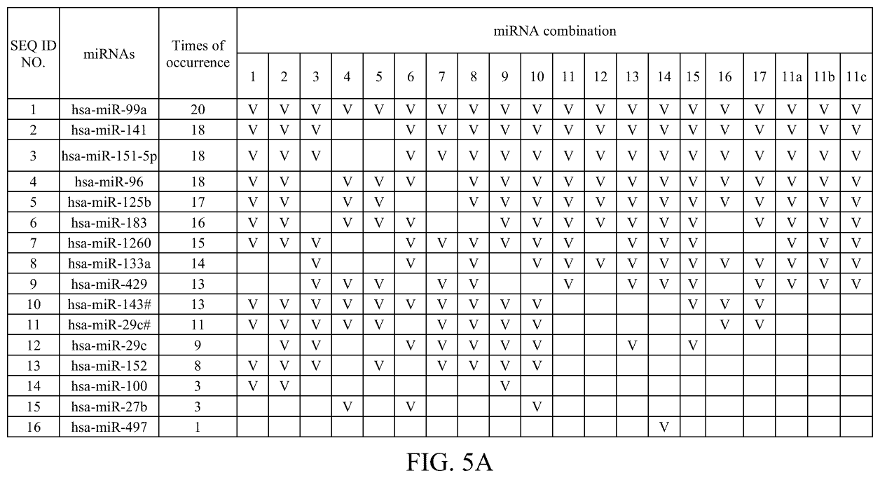

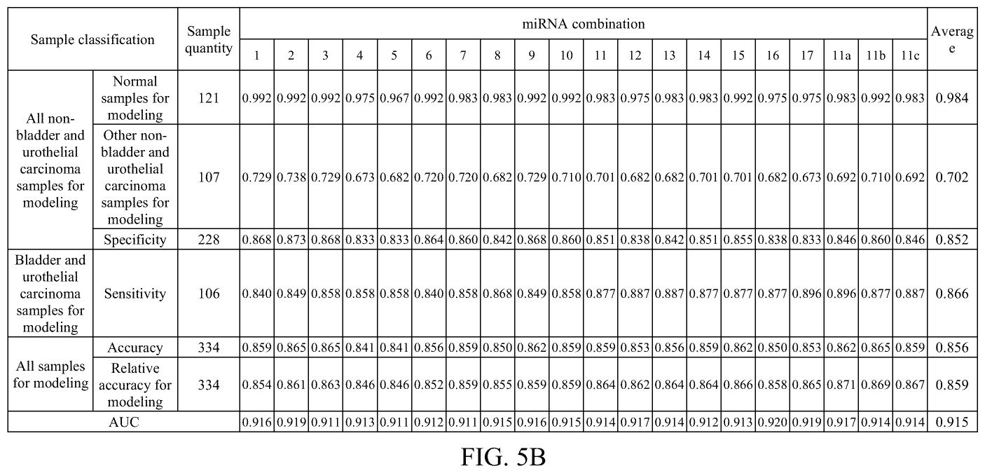

The combinations formed by the markers of the single miRNAs or paired miRNAs that can best distinguish a bladder and urothelial carcinoma group from a non-bladder and urothelial carcinoma group that were selected in the step 2) were verified by a statistical method of SVM to obtain 20 miRNA combinations with optimal sensitivities and specificities (as shown in FIG. 5A and FIG. 5B). The combinations 11, 11a, 11b, and 11c have the same 9 miRNAs but have ratios calculated based on different miRNA pairs. The 334 training samples were verified by using the miRNA combinations selected in the list.

As shown in FIG. 5B, the 334 samples for modeling include 228 samples of the non-bladder and urothelial carcinoma group (including normal samples an samples of other diseases) and 106 samples of the bladder and urothelial carcinoma group. For each sample in the 228 samples of the non-bladder and urothelial carcinoma group, the expression level of each miRNA in a miRNA combination was obtained, a score of an expression pattern of the sample was calculated by using a trained classification algorithm, and the score was compared with a threshold, to draw a conclusion whether the sample pertained to the non-bladder and urothelial carcinoma group. If 198 of the 228 samples were correctly assessed as pertaining to the non-bladder and urothelial carcinoma group, the specificity of the miRNA combination was calculated to be 198/228, i.e., 0.868. Other miRNA combinations were verified similarly.

Similarly, for each sample in the 106 samples of the bladder and urothelial carcinoma group, a score of an expression pattern of the sample was calculated for a miRNA combination, and the score was compared with a threshold, to draw a conclusion whether the sample pertained to the bladder and urothelial carcinoma group. If 89 of the 106 samples were correctly assessed as pertaining to the bladder and urothelial carcinoma group, the sensitivity of the miRNA combination was calculated to be 89/106, i.e., 0.840. Other miRNA combinations were verified similarly.

The accuracy of each miRNA combination was calculated for all samples for modeling. If 198 non-cancer samples and 89 cancer samples were correctly assessed out of the 334 samples by a certain miRNA combination, the accuracy of the miRNA combination was calculated to be (198+89)/334, i.e., 0.859. To eliminate the deviation of the accuracy calculation caused by the imbalance of the distribution of different samples, the relative accuracy of each miRNA combination was also calculated. That is, if a certain miRNA combination had a specificity of 0.868 and a sensitivity of 0.840, the relative accuracy was (0.868+0.840)/2, i.e., 0.854. The relative accuracy is not affected by the imbalance of the quantity of cancer samples and non-cancer samples, so that it can more directly reflect the accuracy of a miRNA combination for sample prediction.

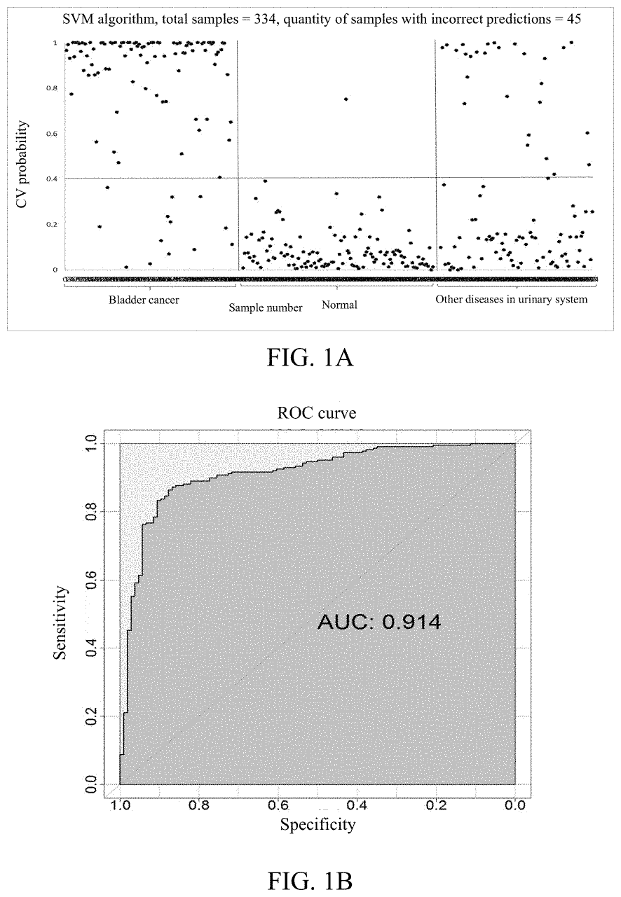

As shown in FIG. 5B, each tested miRNA combination has good sensitivity, specificity, and accuracy. The 20 optimal miRNA combinations have an average sensitivity of 86.9%, an average specificity of 85.2%, an average accuracy of 85.7%, and an average relative accuracy (excluding the effect of the imbalance of the distribution of non-bladder and urothelial carcinoma samples) of 86.0%. For example, as shown in FIG. 1A, the miRNA combination 11b has a sensitivity of 87.7%, a specificity of 86.0%, an accuracy of 86.5%, and an average relative accuracy of 86.9%. As shown in FIG. 1B, the AUC of the ROC curve is 0.914.

These 20 miRNA combinations involve a total of 16 miRNAs. It has been verified that other combinations of these 16 miRNAs can distinguish a bladder and urothelial carcinoma group from a non-bladder and urothelial carcinoma group with similar sensitivity, specificity, and accuracy.

### EXAMPLE 7

### Blind Verification of miRNA Markers Specific to Bladder and Urothelial Carcinoma

339 blind samples were detected by the methods in the examples 2, 3, and 4. A score of an expression pattern of each blind sample was calculated by a statistical method of SVM for the 20 miRNA combinations determined in the example 6, and then the score was compared with a threshold, to draw a conclusion whether the blind sample pertained to the non-bladder and urothelial carcinoma group. After unblinding, the specificity, sensitivity, accuracy, and relative accuracy of each miRNA combination in the prediction of blind samples were calculated by the method in the example 6.

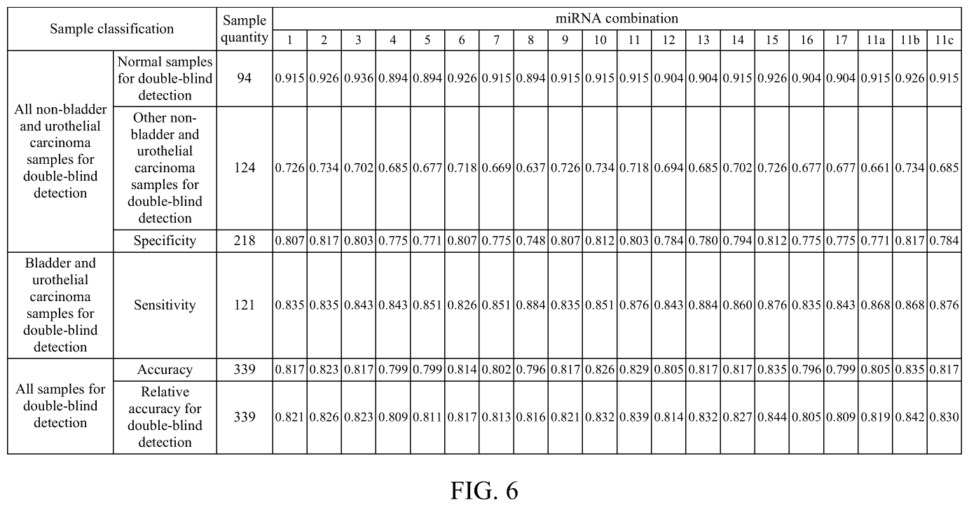

As shown in FIG. 6, in the detection of blind samples of bladder and urothelial carcinoma, each of the selected 20 miRNA combinations has good sensitivity, specificity, accuracy, and relative accuracy. The 20 miRNA combinations have an average sensitivity of 85.4%, an average specificity of 78.1%, an average accuracy of 81.3%, and an average relative accuracy (excluding the effect of the imbalance of the distribution between non-bladder and urothelial carcinoma samples and bladder and urothelial carcinoma samples) of 82.2%. For example, the combination 11b has a sensitivity of 86.8%, a specificity of 81.7%, an accuracy of 83.5%, and an average relative accuracy of 84.2%. The detection results of blind samples fully verified the reliability and stability of the model used and further verified the selected miRNA markers.

It can be learned that the miRNA fingerprints in the present disclosure can efficiently and accurately distinguish bladder and urothelial carcinoma from non-bladder and urothelial carcinoma. Especially, it can greatly distinguish normal samples from bladder and urothelial carcinoma samples.

### EXAMPLE 8

### Core miRNA Composition of miRNA Markers Specific to Bladder and Urothelial Carcinoma

The 20 miRNA combinations obtained in the example 6 include 16 miRNAs. FIG. 7 summarizes the frequencies of these miRNA sequences in the 20 miRNA combinations, the expression levels of these miRNA sequences in bladder and urothelial carcinoma samples and non-bladder and urothelial carcinoma samples, and the changes in the expression levels of these miRNA sequences from cancer samples to non-cancer samples.

As shown in FIG. 7, hsa-miR-99a has a frequency of 100%, hsa-miR-141, hsa-miR-151-5p, and hsa-miR-96 all have a frequency of 90%. The 6 miRNAs of hsa-miR-125b, hsa-miR-183, hsa-miR-1260, hsa-miR-133a, hsa-miR-429, and hsa-miR-143# all have a high frequency (of 65-85%).

The changes in the expression of the 16 miRNAs in bladder and urothelial carcinoma samples and the non-bladder and urothelial carcinoma samples are as follows: As shown in FIG. 7, hsa-miR-96 (dCT −3.35), hsa-miR-151-5p (dCT −3.29), hsa-miR-183 (dCT −3.14), hsa-miR-29c (dCT −2.97), hsa-miR-141 (dCT −2.64), hsa-miR-29c# (dCT −2.37), and hsa-miR-429 (dCT −2.35) all have an expression level significantly increased (with dCT less than −2); hsa-miR-152 (dCT −1.17), hsa-miR-27b (dCT −1.17), and hsa-miR-100 (dCT −1.06)all have an expression level increased (with dCT between −1 and −2); hsa-miR-1260 (dCT −0.79), hsa-miR-497 (dCT −0.67), and hsa-miR-125b (dCT −0.57) all have an expression level slightly increased (with dCT between 0 and −1); hsa-miR-99a (dCT 0) has an expression level unchanged; and hsa-miR-143# (dCT 0.35) and hsa-miR-133a all have an expression level slightly decreased (with dCT between 0 and 1). The CT value reflects the expression levels of miRNAs. A higher CT value indicates a lower expression levels, and a lower CT value indicates a higher expression levels. The dCT value reflects the difference of the expression levels between two samples. From a cancer sample to a normal sample, a positive dCT value indicates that the miRNA expression is low in the cancer sample and is high in the normal sample; and a negative dCT value indicates that the miRNA expression is high in the cancer sample and is low in the normal sample. A greater absolute value of the dCT value indicates a greater difference in the expression of the miRNA between the cancer sample and the normal sample.

As shown in FIG. 7, hsa-miR-96, hsa-miR-151-5p, hsa-miR-183, hsa-miR-29c, hsa-miR-141, hsa-miR-29c#, and hsa-miR-429 all have an expression level in the cancer sample significantly higher than that in the normal sample. This is because they have a negative dCT value with a large absolute value and have a high frequency in the 20 miRNA combinations. The increase of the expression levels of these miRNAs in urine samples is an important indicator of bladder and urothelial carcinoma. Interestingly, hsa-miR-99a has an expression level basically unchanged in bladder and urothelial carcinoma samples and non-bladder and urothelial carcinoma samples, but has a frequency of 100% in the 20 miRNA combinations. Considering that the expression level of hsa-miR-99a is basically unchanged, it may be used as an endogenous reference miRNA in the miRNA combination provided by this application. The expression level of hsa-miR-133a is reduced in cancer samples.

Based on this, hsa-miR-96, hsa-miR-151-5p, hsa-miR-183, hsa-miR-29c, hsa-miR-141, hsa-miR-29c#, hsa-miR-429, hsa-miR-99a, and hsa-miR-133a are an important composition of the miRNA fingerprints for the specificity of bladder and urothelial carcinoma.

It can be learned from FIG. 5A and FIG. 5B that: the combination 12 only includes 7 miRNAs (hsa-miR-99a, hsa-miR-141, hsa-miR-151-5p, hsa-miR-96, hsa-miR-125b, hsa-miR-183, and hsa-miR-133a), and the fingerprint composed of the 7 miRNAs can be used to determine bladder and urothelial carcinoma accurately; and the combinations 11, 11a, 11b, and 11c all only include 9 miRNAs (hsa-miR-99a, hsa-miR-141, hsa-miR-151-5p, hsa-miR-96, hsa-miR-125b, hsa-miR-183, hsa-miR-1260, hsa-miR-133a, and hsa-miR-429), and the fingerprint composed of the 9 miRNAs can be used to determine bladder and urothelial carcinoma more accurately.

### EXAMPLE 9

### Comparative Example

### Core miRNA Composition of miRNA Markers Specific to Bladder and Urothelial Carcinoma

Previous studies reported some miRNAs that may be related to bladder cancer, such as hsa-miR-141, hsa-miR-96, and hsa-miR-133a (referring to Enokida H et al., The role of microRNAs in bladder cancer. Investig Clin Urol 2016; 57 Suppl 1:S60-76.). To compare with the miRNA combination of the present disclosure, the following analysis was carried out:

Analysis 1: Modeling analysis was carried out by only using one miRNA marker hsa-miR-141 (SEQ ID NO: 2), and then double-blind prediction was carried out.

Analysis 2: Modeling analysis was carried out by only using one miRNA marker hsa-miR-96 (SEQ ID NO: 4), and then double-blind prediction was carried out.

Analysis 3: Modeling analysis was carried out by only using one miRNA marker hsa-miR-133a (SEQ ID NO: 8), and then double-blind prediction was carried out.

Analysis 4: Modeling analysis was carried out by only using a combination of three miRNA markers hsa-miR-141, hsa-miR-96, and hsa-miR-133a, and then double-blind prediction was carried out.

Analysis 5: Modeling analysis was carried out by using 113 miRNAs of hsa-miR-141 (SEQ ID NO: 2) and SEQ ID NO: 17 to SEQ ID NO: 128, and a control (as shown in FIG. 3), and then double-blind prediction was carried out. This miRNA combination does not include 15 miRNAs of SEQ ID NOs: 1, and 3-16 (as shown in FIG. 7).

Analysis 6: Modeling analysis was carried out by using 113 miRNAs of hsa-miR-96 (SEQ ID NO: 4) and SEQ ID NO: 17 to SEQ ID NO: 128, and a control (as shown in FIG. 3), and then double-blind prediction was carried out. This miRNA combination does not include 15 miRNAs of SEQ ID NOs: 1-3, and 5-16 (as shown in FIG. 7).

Analysis 7: Modeling analysis was carried out by using 113 miRNAs of hsa-miR-133a (SEQ ID NO: 8) and SEQ ID NO: 17 to SEQ ID NO: 128, and a control (as shown in FIG. 3), and then double-blind prediction was carried out. This miRNA combination does not include 15 miRNAs of SEQ ID NOs: 1-7, and 9-16 (as shown in FIG. 7).

Analysis 8: Modeling analysis was carried out by using 115 miRNAs of hsa-miR-141, hsa-miR-96, hsa-miR-133a, and SEQ ID NO: 17 to SEQ ID NO: 128, and a control (as shown in FIG. 3), and then double-blind prediction was carried out. This miRNA combination does not include 13 miRNAs of SEQ ID NOs: 1, 3, 5-7, and 9-16.

In addition, the results of modeling analysis and double-blind detection by using the miRNA combination 11b (as shown in FIG. 5A) of SEQ ID NOs: 1-9 were compared.

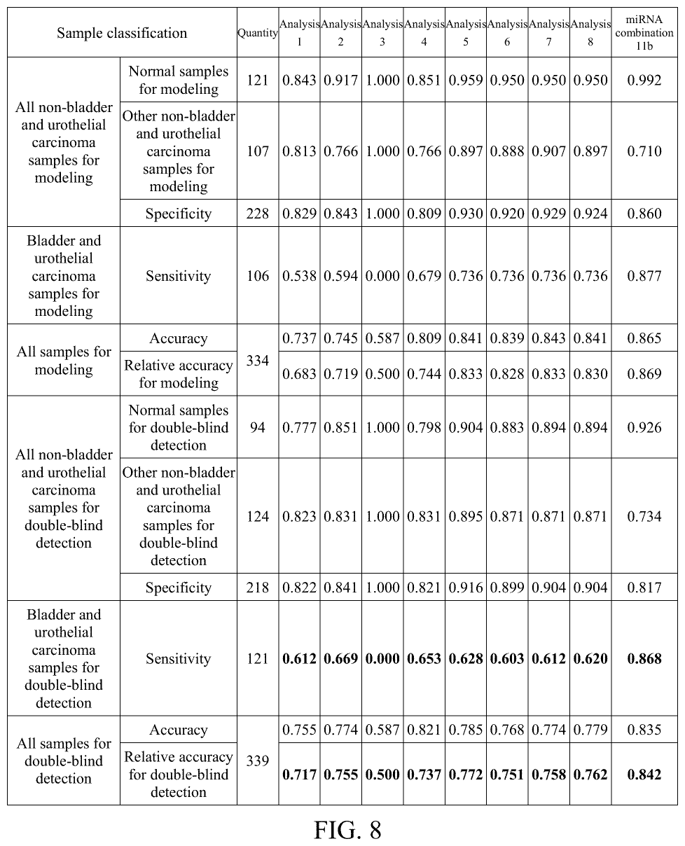

FIG. 8 summarizes results of modeling analysis and double-blind prediction with the optimal accuracy from the analysis 1 to the analysis 8, and the results were compared with results of modeling analysis and double-blind prediction of the miRNA combination 11b.

The training samples and modeling method were the same as in the example 5, except that the miRNA combination of this example was used for modeling.

As shown in FIG. 8, one or all of miRNA markers hsa-miR-141, hsa-miR-96, and hsa-miR-133a were used in the analysis 1 to the analysis 4, which all have an accuracy of prediction of modeling samples and double-blind samples that is significantly less than that of other miRNA combinations. Interestingly, as shown in FIG. 8, 113 or 115 miRNAs were used in the analysis 5 to the analysis 8, which all have a sensitivity of only 0.6 in the prediction of double-blind samples and have a relative accuracy of only 0.7 in the prediction of double-blind samples. However, only 9 miRNAs were used in the miRNA combination of the present disclosure, which has a sensitivity and a relative accuracy both greater than 0.8 in the prediction of the same double-blind samples, more than 30% higher than the analysis 5 to the analysis 8. It indicates that the specific miRNA composition (rather than the quantity of miRNA) of the miRNA combination plays a key role in the accuracy of the prediction results.

In addition, one or all of the three miRNAs (hsa-miR-141, hsa-miR-96, and hsa-miR-133a) that are considered to be related to bladder cancer were used in the miRNA combinations in the analysis 5 to the analysis 8. However, most of the miRNAs (such as SEQ ID NOs: 1, 3, 5-7, and 9-16) in the miRNA combination provided in this application were not included in the miRNA combinations used in the analysis 5 to the analysis 8. Even when they are combined with other miRNAs, that is, combined with miRNAs other than the miRNA fingerprints provided in this application, they cannot provide ideal prediction results for unknown samples. As shown in FIG. 8, in the prediction of double-blind samples, the analysis 5 to the analysis 8 have a sensitivity of only 0.6 and a relative accuracy of only 0.7, which is far lower than the sensitivity and relative accuracy of the miRNA combination provided by the present disclosure in the prediction of double-blind samples. It indicates that the specific miRNA composition of the miRNA combination provided by this application has unexpected advantages in the accuracy of the prediction of bladder and urothelial carcinoma.

### EXAMPLE 10

### Explore Other miRNA Fingerprints Specific to Bladder and Urothelial Carcinoma

To explore other miRNA fingerprints specific to bladder and urothelial carcinoma, 673 urine samples were divided into two parts. The first part was 221 modeling samples including 103 samples of bladder and urothelial carcinoma, 60 samples of normal control and non-urinary system cancer, and 58 samples of non-bladder and urothelial carcinoma. The second part was 487 blind detection samples (without knowing the information of the samples by data analysts) including 143 samples of bladder and urothelial carcinoma, 166 samples of normal control and non-urinary system cancer, and 178 samples of non-bladder and urothelial carcinoma.

The miRNA detection method in this example was the same as in the example 4.

The analysis method in this example was the same as in the example 5.

The quantity of samples for modeling in this example was different from that in the example 6. The quantity of samples for double-blind verification in this example was different from that in the example 7. In this example, 221 modeling samples included 103 samples of bladder and urothelial carcinoma, 60 samples of normal control and non-urinary system cancer, and 58 samples of non-bladder and urothelial carcinoma. The ratio of samples of bladder and urothelial carcinoma:samples of normal control and non-urinary system cancer:samples of non-bladder and urothelial carcinoma was about 1:0.5:0.5. In the example 7, 334 modeling samples included 106 samples of bladder and urothelial carcinoma, 121 samples of normal control and non-urinary system cancer, and 107 samples of non-bladder and urothelial carcinoma. The ratio of samples of bladder and urothelial carcinoma:samples of normal control and non-urinary system cancer:samples of non-bladder and urothelial carcinoma was about 1:1:1.

In this example, modeling analysis was carried out by using 16 miRNAs (hsa-miR-99a, hsa-miR-141, hsa-miR-151-5p, hsa-miR-96, hsa-miR-125b, hsa-miR-183, hsa-miR-1260, hsa-miR-133a, hsa-miR-429, hsa-miR-143#, hsa-miR-29c#, hsa-miR-29c, hsa-miR-152, hsa-miR-100, hsa-miR-27b, and hsa-miR-497) in the 20 miRNA fingerprints determined in the example 6, and then blind prediction was carried out.

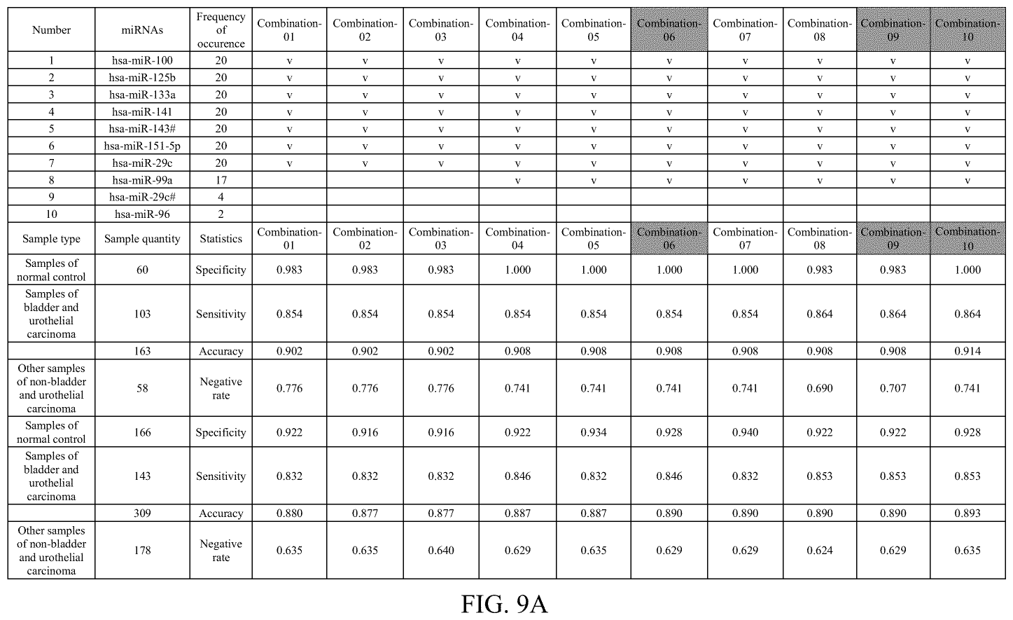

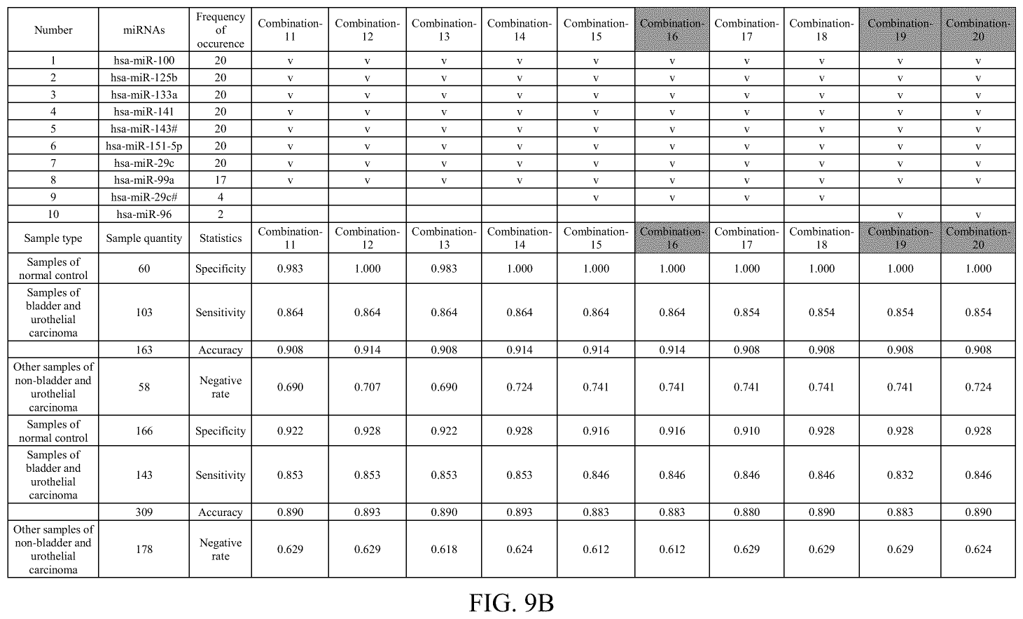

FIG. 9 summarizes 20 miRNA combinations with the optimal accuracy in the blind prediction. The 20 miRNA combinations include 10 miRNAs of hsa-miR-100, hsa-miR-125b, hsa-miR-133a, hsa-miR-141, hsa-miR-143#, hsa-miR-151-5p, hsa-miR-29c, hsa-miR-29c#, hsa-miR-99a, and hsa-miR-96. The combinations 1 to 3 include 7 miRNAs of hsa-miR-100, hsa-miR-125b, hsa-miR-133a, hsa-miR-141, hsa-miR-143#, hsa-miR-151-5p, and hsa-miR-29c. The combinations 4 to 14 include 8 miRNAs of hsa-miR-100, hsa-miR-125b, hsa-miR-133a, hsa-miR-141, hsa-miR-143#, hsa-miR-151-5p, hsa-miR-29c, and hsa-miR-99a. The combinations 15 to 18 include 9 miRNAs of hsa-miR-100, hsa-miR-125b, hsa-miR-133a, hsa-miR-141, hsa-miR-143#, hsa-miR-151-5p, hsa-miR-29c, hsa-miR-99a, and hsa-miR-29c#. The combinations 19 and 20 only include 9 miRNAs of hsa-miR-100, hsa-miR-125b, hsa-miR-133a, hsa-miR-141, hsa-miR-143#, hsa-miR-151-5p, hsa-miR-29c, hsa-miR-99a, and hsa-miR-96. These analysis results show that: 8 miRNAs (hsa-miR-100, hsa-miR-125b, hsa-miR-133a, hsa-miR-141, hsa-miR-143#, hsa-miR-151-5p, hsa-miR-29c, and hsa-miR-99a) can form the most accurate fingerprint specific to bladder and urothelial carcinoma.

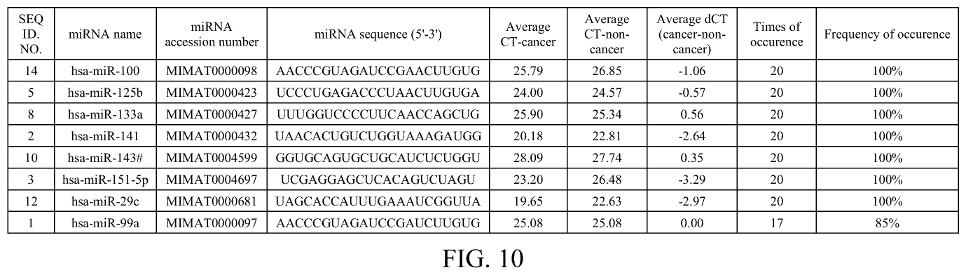

FIG. 10 shows the difference of the expression levels of the 8 miRNAs (hsa-miR-100, hsa-miR-125b, hsa-miR-133a, hsa-miR-141, hsa-miR-143#, hsa-miR-151-5p, hsa-miR-29c, and hsa-miR-99a) between cancer samples and normal samples, and the frequency of the 8 miRNAs in the 20 fingerprints specific to bladder and urothelial carcinoma.

It was verified that the 20 miRNA combinations provided by this application (as shown in FIG. 5) and other combinations of 16 miRNAs (hsa-miR-99a, hsa-miR-141, hsa-miR-151-5p, hsa-miR-96, hsa-miR-125b, hsa-miR-183, hsa-miR-1260, hsa-miR-133a, hsa-miR-429, hsa-miR-143#, hsa-miR-29c#, hsa-miR-29c, hsa-miR-152, hsa-miR-100, hsa-miR-27b, and hsa-miR-497) in the 20 combinations can accurately distinguish samples of bladder and urothelial carcinoma from samples of non-bladder and urothelial carcinoma, especially can distinguish normal samples from samples of bladder and urothelial carcinoma, with high sensitivity and specificity.

Further, the other 20 miRNA combinations provided by this application (as shown in FIG. 9) and 10 miRNAs (hsa-miR-100, hsa-miR-125b, hsa-miR-133a, hsa-miR-141, hsa-miR-143#, hsa-miR-151-5p, hsa-miR-29c, hsa-miR-29c#, hsa-miR-99a, and hsa-miR-96) in the 20 combinations, especially 8 miRNAs therein (hsa-miR-100, hsa-miR-125b, hsa-miR-133a, hsa-miR-141, hsa-miR-143#, hsa-miR-151-5p, hsa-miR-29c, and hsa-miR-99a), can accurately distinguish samples of bladder and urothelial carcinoma from samples of non-bladder and urothelial carcinoma, especially can distinguish normal samples from samples of bladder and urothelial carcinoma, with high sensitivity and specificity.

All the documents mentioned in the present disclosure are incorporated herein by references, just as if each of the documents is individually incorporated by reference. In addition, it should be understood that, after reading the foregoing teaching of the present disclosure, those skilled in the art can make various changes or modifications to the present disclosure, and these equivalent forms also fall within the scope defined by the appended claims of this application.

1. A method for diagnosing whether a test subject suffers from bladder and urothelial carcinoma or is at a high risk of bladder and urothelial carcinoma, comprising:
a) obtaining a test urine sample from the test subject;
b) measuring an expression level of each miRNA in a miRNA combination from the test urine sample, wherein the miRNA combination comprises at least 3 miRNAs selected from a group consisting of: hsa-miR-99a, hsa-miR-141, hsa-miR-151-5p, hsa-miR-96, hsa-miR-125b, hsa-miR-183, hsa-miR-1260, hsa-miR-133a, hsa-miR-429, hsa-miR-143#, hsa-miR-29c#, hsa-miR-29c, hsa-miR-152, hsa-miR-100, hsa-miR-27b, and hsa-miR-497;
c) assessing whether the test subject suffers from bladder and urothelial carcinoma or is at a high risk of bladder and urothelial carcinoma, based on the expression levels of the miRNAs.

2. The method according to claim 1, wherein the miRNA combination comprises at least 3, at least 4, at least 5, at least 6, at least 7, at least 8, at least 9, at least 10, at least 11, at least 12, at least 13, at least 14, at least 15, or at least 16 miRNAs selected from a group consisting of: hsa-miR-99a, hsa-miR-141, hsa-miR-151-5p, hsa-miR-96, hsa-miR-125b, hsa-miR-183, hsa-miR-1260, hsa-miR-133a, hsa-miR-429, hsa-miR-143#, hsa-miR-29c#, hsa-miR-29c, hsa-miR-152, hsa-miR-100, hsa-miR-27b, and hsa-miR-497.

3. The method according to claim 1, wherein the miRNA combination comprises the following 3 miRNAs: hsa-miR-99a, hsa-miR-141, and hsa-miR-151-5p; or comprises the following 3 miRNAs: hsa-miR-100, hsa-miR-141, and hsa-miR-151-5p.

4. The method according to claim 1, wherein the miRNA combination comprises at least the following 4 miRNAs: hsa-miR-99a, hsa-miR-141, hsa-miR-151-5p, and hsa-miR-96; or comprises at least the following 4 miRNAs: hsa-miR-100, hsa-miR-141, hsa-miR-151-5p, and hsa-miR-96.

5. The method according to claim 1, wherein the miRNA combination comprises at least the following 7 miRNAs: hsa-miR-99a, hsa-miR-141, hsa-miR-151-5p, hsa-miR-125b, hsa-miR-133a, hsa-miR-143#, and hsa-miR-29c; or comprises the following 7 miRNAs: hsa-miR-100, hsa-miR-141, hsa-miR-151-5p, hsa-miR-125b, hsa-miR-133a, hsa-miR-143#, and hsa-miR-29c.

6. The method according to claim 1, wherein the miRNA combination comprises any one of miRNA combinations selected from combination 1, combination 2, combination 3, combination 4, combination 5, combination 6, combination 7, combination 8, combination 9, combination 10, combination 11, combination 12, combination 13, combination 14, combination 15, combination 16, combination 17, combination 18, combination 19, combination 20, combination 21, and combination 22, wherein:
1) the combination 1 comprises: hsa-miR-99a, hsa-miR-141, hsa-miR-151-5p, hsa-miR-96, hsa-miR-125b, hsa-miR-183, hsa-miR-1260, hsa-miR-143#, hsa-miR-29c#, hsa-miR-152, and hsa-miR-100;
2) the combination 2 comprises: hsa-miR-99a, hsa-miR-141, hsa-miR-151-5p, hsa-miR-96, hsa-miR-125b, hsa-miR-183, hsa-miR-1260, hsa-miR-143#, hsa-miR-29c#, hsa-miR-29c, hsa-miR-152, and hsa-miR-100;
3) the combination 3 comprises: hsa-miR-99a, hsa-miR-141, hsa-miR-151-5p, hsa-miR-1260, hsa-miR-133a, hsa-miR-429, hsa-miR-143#, hsa-miR-29c#, hsa-miR-29c, and hsa-miR-152; or comprises: hsa-miR-100, hsa-miR-141, hsa-miR-151-5p, hsa-miR-1260, hsa-miR-133a, hsa-miR-429, hsa-miR-143#, hsa-miR-29c#, hsa-miR-29c, and hsa-miR-152;
4) the combination 4 comprises: hsa-miR-99a, hsa-miR-96, hsa-miR-125b, hsa-miR-183, hsa-miR-429, hsa-miR-143#, hsa-miR-29c#, and hsa-miR-27b; or comprises: hsa-miR-100, hsa-miR-96, hsa-miR-125b, hsa-miR-183, hsa-miR-429, hsa-miR-143#, hsa-miR-29c#, and hsa-miR-27b;
5) the combination 5 comprises: hsa-miR-99a, hsa-miR-96, hsa-miR-125b, hsa-miR-183, hsa-miR-429, hsa-miR-143#, hsa-miR-29c#, and hsa-miR-152; or comprises: hsa-miR-100, hsa-miR-96, hsa-miR-125b, hsa-miR-183, hsa-miR-429, hsa-miR-143#, hsa-miR-29c#, and hsa-miR-152;
6) the combination 6 comprises: hsa-miR-99a, hsa-miR-141, hsa-miR-151-5p, hsa-miR-96, hsa-miR-183, hsa-miR-1260, hsa-miR-133a, hsa-miR-143#, hsa-miR-29c, and hsa-miR-27b; or comprises: hsa-miR-100, hsa-miR-141, hsa-miR-151-5p, hsa-miR-96, hsa-miR-183, hsa-miR-1260, hsa-miR-133a, hsa-miR-143#, hsa-miR-29c, and hsa-miR-27b;
7) the combination 7 comprises: hsa-miR-99a, hsa-miR-141, hsa-miR-151-5p, hsa-miR-1260, hsa-miR-429, hsa-miR-143#, hsa-miR-29c#, hsa-miR-29c, and hsa-miR-152; or comprises: hsa-miR-100, hsa-miR-141, hsa-miR-151-5p, hsa-miR-1260, hsa-miR-429, hsa-miR-143#, hsa-miR-29c#, hsa-miR-29c, and hsa-miR-152;
8) the combination 8 comprises: hsa-miR-99a, hsa-miR-141, hsa-miR-151-5p, hsa-miR-96, hsa-miR-125b, hsa-miR-1260, hsa-miR-133a, hsa-miR-429, hsa-miR-143#, hsa-miR-29c#, hsa-miR-29c, and hsa-miR-152; or comprises: hsa-miR-100, hsa-miR-141, hsa-miR-151-5p, hsa-miR-96, hsa-miR-125b, hsa-miR-1260, hsa-miR-133a, hsa-miR-429, hsa-miR-143#, hsa-miR-29c#, hsa-miR-29c, and hsa-miR-152;
9) the combination 9 comprises: hsa-miR-99a, hsa-miR-141, hsa-miR-151-5p, hsa-miR-96, hsa-miR-125b, hsa-miR-183, hsa-miR-1260, hsa-miR-143#, hsa-miR-29c#, hsa-miR-29c, hsa-miR-152, and hsa-miR-100;
10) the combination 10 comprises: hsa-miR-99a, hsa-miR-141, hsa-miR-151-5p, hsa-miR-96, hsa-miR-125b, hsa-miR-183, hsa-miR-1260, hsa-miR-133a, hsa-miR-143#, hsa-miR-29c#, hsa-miR-29c, hsa-miR-152, and hsa-miR-27b; or comprises: hsa-miR-100, hsa-miR-141, hsa-miR-151-5p, hsa-miR-96, hsa-miR-125b, hsa-miR-183, hsa-miR-1260, hsa-miR-133a, hsa-miR-143#, hsa-miR-29c#, hsa-miR-29c, hsa-miR-152, and hsa-miR-27b;
11) the combination 11 comprises: hsa-miR-99a, hsa-miR-141, hsa-miR-151-5p, hsa-miR-96, hsa-miR-125b, hsa-miR-183, hsa-miR-1260, hsa-miR-133a, and hsa-miR-429; or comprises: hsa-miR-100, hsa-miR-141, hsa-miR-151-5p, hsa-miR-96, hsa-miR-125b, hsa-miR-183, hsa-miR-1260, hsa-miR-133a, and hsa-miR-429;
12) the combination 12 comprises: hsa-miR-99a, hsa-miR-141, hsa-miR-151-5p, hsa-miR-96, hsa-miR-125b, hsa-miR-183, and hsa-miR-133a; or comprises: hsa-miR-100, hsa-miR-141, hsa-miR-151-5p, hsa-miR-96, hsa-miR-125b, hsa-miR-183, and hsa-miR-133a;
13) the combination 13 comprises: hsa-miR-99a, hsa-miR-141, hsa-miR-151-5p, hsa-miR-96, hsa-miR-125b, hsa-miR-183, hsa-miR-1260, hsa-miR-133a, hsa-miR-429, and hsa-miR-29c; or comprises: hsa-miR-100, hsa-miR-141, hsa-miR-151-5p, hsa-miR-96, hsa-miR-125b, hsa-miR-183, hsa-miR-1260, hsa-miR-133a, hsa-miR-429, and hsa-miR-29c;
14) the combination 14 comprises: hsa-miR-99a, hsa-miR-141, hsa-miR-151-5p, hsa-miR-96, hsa-miR-125b, hsa-miR-183, hsa-miR-1260, hsa-miR-133a, hsa-miR-429, and hsa-miR-497; or comprises: hsa-miR-100, hsa-miR-141, hsa-miR-151-5p, hsa-miR-96, hsa-miR-125b, hsa-miR-183, hsa-miR-1260, hsa-miR-133a, hsa-miR-429, and hsa-miR-497;
15) the combination 15 comprises: hsa-miR-99a, hsa-miR-141, hsa-miR-151-5p, hsa-miR-96, hsa-miR-125b, hsa-miR-183, hsa-miR-1260, hsa-miR-133a, hsa-miR-429, hsa-miR-143#, and hsa-miR-29c; or comprises: hsa-miR-100, hsa-miR-141, hsa-miR-151-5p, hsa-miR-96, hsa-miR-125b, hsa-miR-183, hsa-miR-1260, hsa-miR-133a, hsa-miR-429, hsa-miR-143#, and hsa-miR-29c;
16) the combination 16 comprises: hsa-miR-99a, hsa-miR-141, hsa-miR-151-5p, hsa-miR-96, hsa-miR-125b, hsa-miR-133a, hsa-miR-143#, and hsa-miR-29c#; or comprises: hsa-miR-100, hsa-miR-141, hsa-miR-151-5p, hsa-miR-96, hsa-miR-125b, hsa-miR-133a, hsa-miR-143#, and hsa-miR-29c#;
17) the combination 17 comprises: hsa-miR-99a, hsa-miR-141, hsa-miR-151-5p, hsa-miR-96, hsa-miR-125b, hsa-miR-183, hsa-miR-133a, hsa-miR-429, hsa-miR-143#, and hsa-miR-29c#; or comprises: hsa-miR-100, hsa-miR-141, hsa-miR-151-5p, hsa-miR-96, hsa-miR-125b, hsa-miR-183, hsa-miR-133a, hsa-miR-429, hsa-miR-143#, and hsa-miR-29c#;
18) the combination 18 comprises: hsa-miR-100, hsa-miR-125b, hsa-miR-133a, hsa-miR-141, hsa-miR-143#, hsa-miR-151-5p, hsa-miR-29c, hsa-miR-29c#, hsa-miR-99a, and hsa-miR-96; and
19) the combination 19 comprises: hsa-miR-100, hsa-miR-125b, hsa-miR-133a, hsa-miR-141, hsa-miR-143#, hsa-miR-151-5p, hsa-miR-29c, and hsa-miR-99a;
20) the combination 20 comprises: hsa-miR-100, hsa-miR-125b, hsa-miR-133a, hsa-miR-141, hsa-miR-143#, hsa-miR-151-5p, hsa-miR-29c, hsa-miR-99a, and hsa-miR-29c#;
21) the combination 21 comprises: hsa-miR-100, hsa-miR-125b, hsa-miR-133a, hsa-miR-141, hsa-miR-143#, hsa-miR-151-5p, hsa-miR-29c, hsa-miR-99a, and hsa-miR-96; and
22) the combination 22 comprises: hsa-miR-141, hsa-miR-151-5p, hsa-miR-143#, hsa-miR-125b, hsa-miR-29c, hsa-miR-100, and hsa-miR-133a; or comprises: hsa-miR-141, hsa-miR-151-5p, hsa-miR-143#, hsa-miR-125b, hsa-miR-29c, hsa-miR-99a, and hsa-miR-133a.

7. The method according to claim 1, wherein the expression levels of the miRNAs is normalized to an endogenous reference.

8. The method according to claim 7, wherein the endogenous reference comprises one or more miRNAs from the miRNA combination.

9. The method according to claim 8, wherein the endogenous reference comprises hsa-miR-99a or hsa-miR-100.

10. The method according to claim 1, further comprising enriching RNA from the test urine sample before measuring the expression levels of the miRNAs.

11. The method according to claim 1, wherein the step c) further comprises calculating an expression pattern of the miRNA combination, based on the expression levels of the miRNAs.

12. The method according to claim 11, wherein the expression pattern is calculated through a function or model related to the expression level of each miRNA and a decision weight of each miRNA for sample states, and the function or model is calculated by a classification algorithm.

13. The method according to claim 12, wherein the classification algorithm is subjected to at least one training of a positive training data set and a negative training data set to provide one or more decision weights of the function or model for calculating the expression pattern, the positive training data set comprises the expression level of each miRNA in the miRNA combination from a plurality of urine samples of subjects with bladder and urothelial carcinoma, and the negative training data set comprises the expression level of each miRNA in the miRNA combination from a plurality of urine samples of subjects without bladder and urothelial carcinoma.

14. The method according to claim 13, wherein the training comprises training with the positive training data set and the negative training data set to provide one or more decision weights of the function or model for calculating a positive expression pattern and one or more decision weights of the function or model for calculating a negative expression pattern.

15. The method according to claim 14, wherein the expression pattern is a score between 0 and 1.

16. The method according to claim 15, wherein a threshold is determined based on a score of the positive expression pattern and a score of the negative expression pattern, wherein the threshold is able to distinguish the positive expression pattern from the negative expression pattern.

17. The method according to claim 16, further comprising comparing a score of the expression pattern calculated based on the expression levels of the miRNAs in the test urine sample with the threshold to assess whether the test subject suffers from bladder and urothelial carcinoma or is at a high risk of bladder and urothelial carcinoma.

18. The method according to claim 17, wherein the threshold is a value between 0.2 and 0.8, and if the score of the expression pattern is greater than the threshold, the test subject is assessed as suffering from bladder and urothelial carcinoma or being at a high risk of bladder and urothelial carcinoma.

19. The method according to claim 18, wherein the threshold is 0.4.

20. The method according to claim 1, further comprising administering a bladder and urothelial carcinoma therapy to the test subject assessed as suffering from bladder and urothelial carcinoma or being at a high risk of bladder and urothelial carcinoma in the step c).

21. The method according to claim 20, wherein the bladder and urothelial carcinoma therapy comprises chemotherapy, radiotherapy, immunotherapy, surgery, or anti-cancer drug therapy.

22. A set of isolated oligonucleotides comprising a hybridization region, wherein the hybridization region in each of the oligonucleotides can hybridize with a corresponding miRNA in a miRNA combination or a complement sequence thereof, and the miRNA combination comprises:
1) at least 3, at least 4, at least 5, at least 6, at least 7, at least 8, at least 9, at least 10, at least 11, at least 12, at least 13, at least 14, at least 15, or at least 16 miRNAs selected from a group consisting of: hsa-miR-99a, hsa-miR-141, hsa-miR-151-5p, hsa-miR-96, hsa-miR-125b, hsa-miR-183, hsa-miR-1260, hsa-miR-133a, hsa-miR-429, hsa-miR-143#, hsa-miR-29c#, hsa-miR-29c, hsa-miR-152, hsa-miR-100, hsa-miR-27b, and hsa-miR-497;
2) at least 3, at least 4, at least 5, at least 6, at least 7, at least 8, at least 9, at least 10, at least 11, at least 12, at least 13, at least 14, or at least 15 miRNAs selected from a group consisting of: hsa-miR-99a, hsa-miR-141, hsa-miR-151-5p, hsa-miR-96, hsa-miR-125b, hsa-miR-183, hsa-miR-1260, hsa-miR-133a, hsa-miR-429, hsa-miR-143#, hsa-miR-29c#, hsa-miR-29c, hsa-miR-152, hsa-miR-100, and hsa-miR-27b;
3) at least 3, at least 4, at least 5, at least 6, at least 7, at least 8, at least 9, at least 10, at least 11, at least 12, at least 13, or at least 14 miRNAs selected from a group consisting of: hsa-miR-99a, hsa-miR-141, hsa-miR-151-5p, hsa-miR-96, hsa-miR-125b, hsa-miR-183, hsa-miR-1260, hsa-miR-133a, hsa-miR-429, hsa-miR-143#, hsa-miR-29c#, hsa-miR-29c, hsa-miR-152, and hsa-miR-100;
4) at least 3, at least 4, at least 5, at least 6, at least 7, at least 8, at least 9, at least 10, at least 11, at least 12, or at least 13 miRNAs selected from a group consisting of: hsa-miR-99a (or hsa-miR-100), hsa-miR-141, hsa-miR-151-5p, hsa-miR-96, hsa-miR-125b, hsa-miR-183, hsa-miR-1260, hsa-miR-133a, hsa-miR-429, hsa-miR-143#, hsa-miR-29c#, hsa-miR-29c, and hsa-miR-152;
5) at least 3, at least 4, at least 5, at least 6, at least 7, at least 8, at least 9, at least 10, at least 11, or at least 12 miRNAs selected from a group consisting of: hsa-miR-99a (or hsa-miR-100), hsa-miR-141, hsa-miR-151-5p, hsa-miR-96, hsa-miR-125b, hsa-miR-183, hsa-miR-1260, hsa-miR-133a, hsa-miR-429, hsa-miR-143#, hsa-miR-29c#, and hsa-miR-29c;
6) at least 3, at least 4, at least 5, at least 6, at least 7, at least 8, at least 9, at least 10, or at least 11 miRNAs selected from a group consisting of: hsa-miR-99a (or hsa-miR-100), hsa-miR-141, hsa-miR-151-5p, hsa-miR-96, hsa-miR-125b, hsa-miR-183, hsa-miR-1260, hsa-miR-133a, hsa-miR-429, hsa-miR-143#, and hsa-miR-29c#;
7) at least 3, at least 4, at least 5, at least 6, at least 7, at least 8, at least 9, or at least 10 miRNAs selected from a group consisting of: hsa-miR-99a (or hsa-miR-100), hsa-miR-141, hsa-miR-151-5p, hsa-miR-96, hsa-miR-125b, hsa-miR-183, hsa-miR-1260, hsa-miR-133a, hsa-miR-429, and hsa-miR-143#;
8) at least 3, at least 4, at least 5, at least 6, at least 7, at least 8, at least 9, or at least 10 miRNAs selected from a group consisting of: hsa-miR-100, hsa-miR-125b, hsa-miR-133a, hsa-miR-141, hsa-miR-143#, hsa-miR-151-5p, hsa-miR-29c, hsa-miR-29c#, hsa-miR-99a, and hsa-miR-96;
9) at least 3, at least 4, at least 5, at least 6, at least 7, at least 8, or at least 9 miRNAs selected from a group consisting of: hsa-miR-99a (or hsa-miR-100), hsa-miR-141, hsa-miR-151-5p, hsa-miR-96, hsa-miR-125b, hsa-miR-183, hsa-miR-1260, hsa-miR-133a, and hsa-miR-429;
10) at least 3, at least 4, at least 5, at least 6, at least 7, at least 8, or at least 9 miRNAs selected from a group consisting of: hsa-miR-100, hsa-miR-125b, hsa-miR-133a, hsa-miR-141, hsa-miR-143#, hsa-miR-151-5p, hsa-miR-29c, hsa-miR-99a, and hsa-miR-29c#;
11) at least 3, at least 4, at least 5, at least 6, at least 7, at least 8, or at least 9 miRNAs selected from a group consisting of: hsa-miR-100, hsa-miR-125b, hsa-miR-133a, hsa-miR-141, hsa-miR-143#, hsa-miR-151-5p, hsa-miR-29c, hsa-miR-99a, and hsa-miR-96;
12) at least 3, at least 4, at least 5, at least 6, at least 7, or at least 8 miRNAs selected from a group consisting of: hsa-miR-99a (or hsa-miR-100), hsa-miR-141, hsa-miR-151-5p, hsa-miR-96, hsa-miR-125b, hsa-miR-183, hsa-miR-1260, and hsa-miR-133a;
13) at least 3, at least 4, at least 5, at least 6, at least 7, or at least 8 miRNAs selected from a group consisting of: hsa-miR-99a, hsa-miR-141, hsa-miR-151-5p, hsa-miR-143#, hsa-miR-125b, hsa-miR-29c, hsa-miR-100, and hsa-miR-133a;
14) at least 3, at least 4, at least 5, at least 6, or at least 7 miRNAs selected from a group consisting of: hsa-miR-99a (or hsa-miR-100), hsa-miR-141, hsa-miR-151-5p, hsa-miR-96, hsa-miR-125b, hsa-miR-183, and hsa-miR-1260;
15) at least 3, at least 4, at least 5, at least 6, or at least 7 miRNAs selected from a group consisting of: hsa-miR-141, hsa-miR-151-5p, hsa-miR-143#, hsa-miR-125b, hsa-miR-29c, hsa-miR-100 (or hsa-miR-99a), and hsa-miR-133a;
16) at least 3, at least 4, at least 5, or at least 6 miRNAs selected from a group consisting of: hsa-miR-99a (or hsa-miR-100), hsa-miR-141, hsa-miR-151-5p, hsa-miR-96, hsa-miR-125b, and hsa-miR-183;
17) at least 3, at least 4, or at least 5 miRNAs selected from a group consisting of: hsa-miR-99a (or hsa-miR-100), hsa-miR-141, hsa-miR-151-5p, hsa-miR-96, and hsa-miR-125b; or
18) at least 3, or at least 4 miRNAs selected from a group consisting of: hsa-miR-99a (or hsa-miR-100), hsa-miR-141, hsa-miR-151-5p, and hsa-miR-96.

23. A miRNA detecting chip, comprising the isolated oligonucleotides according to claim 22 immobilized on a solid support.

24. A kit for detecting a miRNA combination, comprising the isolated oligonucleotides according to claim 22.

25. A method of screening a drug candidate for treating bladder and urothelial carcinoma, comprising the following steps:
a) measuring an expression level of each miRNA in a miRNA combination from bladder and urothelial carcinoma cells in an experimental group to provide the expression level of the experimental group, and calculating the expression pattern of the miRNA combination of the experimental group based on the expression level of the experimental group to provide the expression pattern of the experimental group, wherein the miRNA combination comprises at least 3 miRNAs selected from a group consisting of: hsa-miR-99a, hsa-miR-141, hsa-miR-151-5p, hsa-miR-96, hsa-miR-125b, hsa-miR-183, hsa-miR-1260, hsa-miR-133a, hsa-miR-429, hsa-miR-143#, hsa-miR-29c#, hsa-miR-29c, hsa-miR-152, hsa-miR-100, hsa-miR-27b, and hsa-miR-497; and the bladder and urothelial carcinoma cells in the experimental group are treated with the drug candidate;
b) measuring the expression level of each miRNA in the miRNA combination from bladder and urothelial carcinoma cells in a control group to provide the expression level of the control group, and calculating the expression pattern of the miRNA combination of the control group based on the expression level of the control group to provide the expression pattern of the control group, wherein the bladder and urothelial carcinoma cells in the control group are not treated with the drug candidate; and
c) comparing the expression patterns between the experimental group and the control group to determine whether there is a significant difference.
